SEQUENCE LISTING (6 sequences)

SEQ ID NO: 1 - protein, 219 aa, synthetic construct
  REGION 1..219: note = 0041-LC1
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 447 aa, synthetic construct
  REGION 1..447: note = 0041-HC1
VQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 3 - protein, 215 aa, synthetic construct
  REGION 1..215: note = 0041-LC2
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 4 - protein, 228 aa, synthetic construct
  REGION 1..228: note = 0041-Fab
SMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 694 aa, synthetic construct
  REGION 1..694: note = 0041-HC2
VQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

(19) United States
(12) Patent Application Publication
(10) Pub. No.: US 2026/0159601 A1
(43) Pub. Date: Jun. 11, 2026

(54) METHODS OF TREATMENT

(71) Applicant: Hoffmann-La Roche Inc., Little Falls, NJ (US)

(72) Inventors: Hans Peter GRIMM, Basel (CH); Niels JANSSEN, Basel (CH); Robert MADER NOPPEL, Binningen (CH); Vanessa Lea SCHUMACHER, Efringen-Kirchen (DE); Stefan Dieter STURM, Oberwil (CH); Alain TISSOT-DAGUETTE, München (DE)

(73) Assignee: Hoffmann-La Roche Inc., Little Falls, NJ (US)

(21) Appl. No.: 19/539,771

(22) Filed: Feb. 13, 2026

Related U.S. Application Data
(63) Continuation of application No. PCT/EP2024/072702, filed on Aug. 12, 2024.

(30) Foreign Application Priority Data
Aug. 14, 2023 (WO) PCT/EP2023/072409

(51) Int. Cl. C07K 16/28; A61K 39/00

(52) U.S. Cl. CPC C07K 16/2887; C07K 16/2881; A61K 2039/505; C07K 2317/24; C07K 2317/31; C07K 2317/55; C07K 2317/73; C07K 2317/77; C07K 2319/00

(57) ABSTRACT

Herein is reported the use of RG6035 as a medicament in the treatment of multiple sclerosis, wherein RG6035 is administered at a dose of 140-210 mg.

The current invention is in the field of treatment of a disease. In more detail, herein is reported amongst other things a dosing scheme for the treatment of multiple sclerosis using a brain-penetrating anti-CD20 antibody.

### BACKGROUND OF THE INVENTION

Multiple sclerosis (MS) is a chronic, inflammatory, demyelinating, and degenerative disease of the central nervous system (CNS) affecting approximately 2.5 million patients worldwide. MS creates a neurodegenerative environment that leads to accumulation of significant motor, sensory and cognitive disabilities [1,2] and is the most common inflammatory neurological disease in young adults.[3]

Over the past 15 years, the pathogenesis of MS has been strongly linked to B-cells. Key players in humoral immunity, and bridging the divide between the innate and adaptive immune systems, B-cells play an essential role in counterattacks against pathogens and foreign antigens through antibody production.[4] However, B-cell dysregulation has been identified as a stimulus for several autoimmune diseases, including rheumatoid arthritis, systemic lupus erythematosus and myasthenia gravis.[5] Although there is currently no specific antibody that can be pinpointed as a hallmark of MS, research has started to characterise distribution and function of B-cells in patients with MS.[5] Beyond antibody generation, B-cells may also contribute to pathology by stimulating T-cell responses as antigen-presenting cells, or secrete pro-inflammatory cytokines. In addition, memory B-cells, which are thought to be the key player in MS, are subject to infection by Epstein Barr Virus, which is thought to be a key contributor to MS pathology [47].

CD20-positive (CD20+) B-cells have been described in several sub-compartments of the CNS in patients with MS, including the cerebrospinal fluid (CSF), parenchyma, meninges and perivascular space.[4,6,7,8] In CSF, the presence of oligoclonal IgG bands in over 90% of patients with MS, as well as cortical demyelination associated with meningeal inflammation, indicate that B-cells become resident in CNS compartments early in the disease.[4,9] Under sustained inflammation, B-cells can infiltrate the immune-privileged CNS from the blood, especially if there is a breakdown of the blood-brain-barrier (BBB).[10]

Delivery of drugs to the brain remains a major challenge in CNS drug development. The BBB is a physical, metabolic and transport barrier that tightly controls movement of substances from blood to neural tissues and vice versa, ensuring CNS homeostasis but limiting delivery of therapeutic monoclonal antibodies (mAbs), such as the anti-CD20 therapies, obinutuzumab, ocrelizumab and ofatumumab.[11-13] As a result, currently available anti-CD20 therapies address peripheral B-cells and efficiently prevent acute relapsing-remitting MS (RRMS) attacks (relapses). Anti-CD20 therapies have limited access to CNS B-cells, which is believed to limit their effect on disease progression. In order to deplete compartmentalized B-cells in the brain, a clinical candidate would need to substantially cross the BBB and kill B-cells in an effector-function independent manner.

Therapeutic antibodies in brain shuttle format are reported, e.g. in WO 2017/055542.

### SUMMARY OF THE INVENTION

The current invention is based, at least in part, on the use of a specific brain shuttle-CD20 (RG6035) construct. RG6035 used in the methods of the current invention is a bispecific modular fusion protein of a human transferrin receptor 1 (TfR1)-directed brain shuttle (a TfR1-binding antigen-binding fragment (Fab)) and a paratope of the anti-CD20 antibody, obinutuzumab. TfR1 is abundant at the BBB and is a target for enabling receptor-mediated transport of large molecules across the BBB, while obinutuzumab has a unique mechanism of action: inducing Fc-effector-independent, non-apoptotic, direct B-cell death upon binding CD20.[14-21] RG6035 has the function to (fully) deplete brain-localized B-cells.

The current invention is based, at least in part, on the finding that a PK/PD model for RG6035 based on non-clinical data can be used to estimate the pharmacologically active/therapeutically efficient, systemic RG6035 exposure. In more detail, it has been found that data observed in blood can be used as a surrogate to model the human effective exposure.

In more detail, the current invention encompasses at least the following embodiments
1. RG6035 for use as a medicament in the treatment of multiple sclerosis, wherein RG6035 is administered at a dose of 140-210 mg.
2. RG6035 for use in the treatment of multiple sclerosis, wherein RG6035 is administered at a dose of 140-210 mg.
3. The use according to any one of embodiments 1 to 2, wherein the multiple sclerosis is primary progressive multiple sclerosis or secondary progressive multiple sclerosis.
4. The use according to any one of embodiments 1 to 3, wherein the dose is effective to deplete more than 95% of B-cells in the CSF of the subject that has been administered RG6035.
5. The use according to embodiment 4, wherein the depletion is achieved within 8 weeks after start of the administration.
6. The use according to any one of embodiments 1 to 5, wherein the concentration of RG6035 in the CSF is 1% or more of the serum concentration of RG6035.
7. The use according to any one of embodiments 1 to 6, wherein the concentration of RG6035 in the CSF is 0.055 g/mL or higher.
8. The use according to any one of claims 4 to 7, wherein the B-cells are CD19 positive B-cells.
9. The use according to any one of embodiments 1 to 8, wherein RG6035 is administered at a second dose after 95% or more of the B-cells have been depleted from the CSF to maintain the B-cell depletion in the CSF at said level.
10. The use according to any one of embodiments 4 to 9, wherein the B-cell depletion is determined compared to the B-cell number in the CSF prior to the first administration of RG6035.
11. The use according to any one of embodiments 1 to 10, wherein the administration is once every four weeks.
12. The use according to any one of embodiments 1 to 10, wherein the administration is started as once every week and changed to once every four weeks after 95% or more of the B-cells have been depleted from the CSF to maintain the B-cell depletion in the CSF at said level.
13. The use according to any one of embodiments 1 to 12, wherein the administration of RG6035 is intravenously or subcutaneously.
14. The use according to any one of embodiments 1 to 13, wherein the administration of RG6035 is subcutaneously.
15. The use according to any one of embodiments 1 to 14, wherein RG6035 comprises a first polypeptide with the amino acid sequence of SEQ ID NO: 01, a second polypeptide with the amino acid sequence of SEQ ID NO: 02, a third polypeptide with the amino acid sequence of SEQ ID NO: 03 and a fourth polypeptide with the amino acid sequence of SEQ ID NO: 05.

In addition to the various embodiments depicted and claimed, the disclosed subject matter is also directed to other embodiments having other combinations of the features disclosed or claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### DETAILED DESCRIPTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless otherwise defined herein the term “comprising of” shall include the term “consisting of”.

The term “about” as used herein in connection with a specific value (e.g. temperature, concentration, time and others) shall refer to a variation of +/−1% of the specific value that the term “about” refers to.

The “blood-brain-barrier” or “BBB” refers to the physiological barrier between the peripheral blood circulation and the brain and spinal cord that is formed by tight junctions within the brain capillary endothelial plasma membranes, creating a tight barrier that restricts the transport of molecules into the brain, even very small molecules such as urea (60 Daltons). The BBB within the brain, the blood-spinal-cord-barrier within the spinal cord, and the blood-retinal-barrier within the retina are contiguous capillary barriers within the CNS, and are herein collectively referred to as the blood-brain-barrier or BBB. The BBB also encompasses the blood-CSF-barrier (choroid plexus) where the barrier is comprised of ependymal cells rather than capillary endothelial cells.

The terms “anti-human CD20 antibody” and “antibody specifically binding to human CD20” refer to an antibody that is capable of binding human CD20 with sufficient affinity such that the antibody is useful as a diagnostic and/or therapeutic agent in targeting CD20.

The “CD20” antigen is an approx. 35 kDa, non-glycosylated phosphoprotein found on the surface of greater than 90% of B-cells from peripheral blood or lymphoid organs. CD20 is expressed during early pre-B-cell development and remains until plasma cell differentiation. CD20 is present on both normal B-cells as well as malignant B-cells. Other names for CD20 in the literature include “B-lymphocyte-restricted antigen” and “Bp35” and “MS4A1”.[46] The CD20 antigen is described in Clark et al. Proc. Natl. Acad. Sci USA 82 (1985) 1766, for example. See also SEQ ID NO: 06.

An “autoimmune disease” herein is a non-malignant disease or disorder arising from and directed against an individual's own tissues. Examples of autoimmune diseases or disorders include, but are not limited to, multiple sclerosis.

An “antagonist” is a molecule that upon binding to a B-cell surface marker destroys, kills or depletes B-cells in a mammal and/or interferes with one or more B-cell functions, e.g. by reducing or preventing a humoral response elicited by the B-cell. The antagonist is able to deplete B-cells (i.e. reduce circulating B-cell numbers or levels) in a mammal treated therewith. Such depletion may be achieved via various mechanisms such antibody-dependent cell-mediated cytotoxicity (ADCC) and/or complement dependent cytotoxicity (CDC), inhibition of B-cell proliferation and/or induction of direct B-cell death (e.g. via apoptosis).

Antagonists which “induce apoptosis” are those which induce programmed cell death, e.g. of a B-cell, as determined by standard apoptosis assays, such as binding of Annexin V, fragmentation of DNA, cell shrinkage, dilation of endoplasmic reticulum, cell fragmentation, and/or formation of membrane vesicles (called apoptotic bodies).

An antagonist “which binds” an antigen of interest, e.g. a B-cell surface marker, is a compound capable of binding that antigen with sufficient affinity and/or avidity such that the antagonist is useful as a therapeutic agent for targeting a cell expressing the antigen.

The “central nervous system” or “CNS” refers to the complex of nerve tissues that control bodily function, and includes the brain and spinal cord.

A “blood-brain-barrier receptor” (BBBR) is an extracellular membrane-linked receptor protein expressed on brain endothelial cells that is capable of transporting molecules across the BBB or can be used to transport exogenous administered molecules. Examples of BBBR include the transferrin receptor 1 (TfR1).

The “transferrin receptor 1” (“TfR1”) is a transmembrane glycoprotein (with a molecular weight of about 180,000 Da) composed of two disulphide-bonded sub-units (each of apparent molecular weight of about 90,000 Da) involved in iron uptake in vertebrates. In certain embodiments of all aspects and embodiments of the current invention, the TfR1 as mentioned herein is human TfR1 comprising the amino acid sequence as in Schneider et al. (Nature 311 (1984) 675-678), for example.

A “multispecific antibody” denotes an antibody having binding specificities for at least two different antigens. Exemplary multispecific antibodies may bind both a BBBR and a brain antigen. Multispecific antibodies can be prepared as full-length antibodies or antibody fragments (e.g. F(ab′) 2 bispecific antibodies) or combinations thereof (e.g. full length antibody plus additional scFv or Fab fragments). Engineered antibodies with two, three or more (e.g. four) functional antigen binding sites have also been reported (see, e.g., US 2002/0004587).

The term “antibody” herein is used to encompass various antibody structures, including but not limited to monoclonal antibodies and multispecific antibodies (e.g., bispecific antibodies) so long as they exhibit the desired antigen-binding activity.

The term “antibody-dependent cellular cytotoxicity (ADCC)” is a function mediated by Fc receptor binding and refers to lysis of target cells by an antibody in the presence of effector cells. ADCC can be measured by the treatment of a preparation of CD19 expressing erythroid cells (e.g. K562 cells expressing recombinant human CD19) with an antibody in question in the presence of effector cells such as freshly isolated peripheral blood mononuclear cells (PBMCs) or purified effector cells from buffy coats, like monocytes or natural killer (NK) cells. Target cells are labeled with Cr⁵¹ and subsequently incubated with the antibody in question. The labeled cells are incubated with effector cells and the supernatant is analyzed for released Cr⁵¹.

Controls include the incubation of the target endothelial cells with effector cells but without the antibody. The capacity of the antibody in question to induce the initial steps mediating ADCC is investigated by measuring their binding to Fcγ receptors expressing cells, such as cells, recombinantly expressing FcγRI and/or FcγRIIA or NK cells (expressing essentially FcγRIIIA).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, gamma, epsilon and miu, respectively.

The term “complement-dependent cytotoxicity (CDC)” refers to lysis of cells induced by an antibody in question in the presence of complement. CDC can be measured by the treatment of CD19 expressing human endothelial cells with an antibody in question in the presence of complement. The cells are labeled with calcein. CDC is found if the antibody in question induces lysis of 20% or more of the target cells at a concentration of 30 μg/ml. Binding to the complement factor C1q can be measured in an ELISA. In such an assay, an ELISA plate is coated with concentration ranges of the antibody in question, to which purified human C1q or human serum is added. C1q binding is detected by an antibody directed against C1q followed by a peroxidase-labeled conjugate. Detection of binding (maximal binding Bₘₐₓ) is measured as optical density at 405 nm (OD405) for peroxidase substrate ABTS® (2,2′-azino-di-[3-ethylbenzthiazoline-6-sulfonate (6)]).

“Effector function” refers to those biological activities attributable to the Fc-region of an antibody, which vary with the antibody class. Examples of antibody effector functions include C1q binding and complement dependent cytotoxicity (CDC); Fc receptor binding; antibody-dependent cell-mediated cytotoxicity (ADCC); phagocytosis; down regulation of cell surface receptors (e.g. B-cell receptor); and B-cell activation.

Fc receptor binding dependent effector functions can be mediated by the interaction of the Fc-region of an antibody with Fc receptors (FcRs), which are specialized cell surface receptors on hematopoietic cells and their descendants. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 103-fold and eliminated the human monocyte response to antibody-sensitized red blood cells (Armour, K. L., et al., Eur. J. Immunol. 29 (1999) 2613-2624);
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat);
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g, for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al., J. Biol. Chem. 276 (2001) 6591-6604.

An “effective amount” of an agent, e.g., a pharmaceutical formulation, refers to an amount effective, at dosages and for periods of time necessary, to achieve the desired therapeutic or prophylactic result.

The term “Fc receptor” as used herein refers to activation receptors characterized by the presence of a cytoplasmic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA. The term “no binding of FcγR” denotes that at an antibody concentration of 10 μg/ml the binding of an antibody in question to NK cells is 10% or less of the binding found for anti-OX40L antibody LC.001 as reported in WO 2006/029879.

While IgG4 shows reduced FcR binding, antibodies of other IgG subclasses show strong binding. However, Pro238, Asp265, Asp270, Asn297 (loss of Fc carbohydrate), Pro329 and 234, 235, 236 and 237 Ile253, Ser254, Lys288, Thr307, Gln311, Asn434, and His435 are residues which provide if altered also reduce FcR binding (Shields, R. L., et al., J. Biol. Chem. 276 (2001) 6591-6604; Lund, J., et al., FASEB J. 9 (1995) 115-119; Morgan, A., et al., Immunology 86 (1995) 319-324; and EP 0 307 434).

The term “Fc-region” herein is used to define a C-terminal region of an immunoglobulin heavy chain that contains at least a portion of the constant region. The term includes native sequence Fc-regions and variant Fc-regions. A human IgG heavy chain Fc-region extends from Cys226, or from Pro230, to the carboxyl-terminus of the heavy chain. However, the C-terminal lysine (Lys447) of the Fc-region may or may not be present.

The Fc-region of an antibody is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R. and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat). Antibodies of subclass IgG1, IgG2 and IgG3 usually show complement activation, C1q binding and C3 activation, whereas IgG4 do not activate the complement system, do not bind C1q and do not activate C3.

An “Fc-region of an antibody” is a term well known to the skilled artisan and defined on the basis of papain cleavage of antibodies. The Fc-region of RG6035 is of the human IgG1 subclass comprising the effector function eliminating mutations L234A, L235A and P329G (numbering according to EU index of Kabat).

The term “full-length antibody” is used herein to refer to an antibody having a structure substantially similar to a native antibody structure or having heavy chains that contain an Fc-region as defined herein. A “full-length antibody” is an antibody that comprises the antigen-binding variable regions VH and VL as well as a light chain constant domain (CL) and heavy chain constant domains, CH1, CH2 and CH3. The constant domains may be native sequence constant domains (e.g. human native sequence constant domains) or amino acid sequence variants thereof. In more detail, a full-length antibody comprises two antibody light chains (each comprising a variable domain and a constant domain) and two antibody heavy chains (each comprising a variable domain, a hinge region and three constant domains including the CH2 and CH3 domains). The C-terminal amino acid residues K or GK may be present or not independently of each other in the two antibody heavy chains of a full-length antibody.

The terms “host cell”, “host cell line”, and “host cell culture” are used interchangeably and refer to cells into which one or more exogenous nucleic acid(s) has (have) been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “humanized” antibody refers to a chimeric antibody comprising amino acid residues from non-human hypervariable regions and amino acid residues from human frameworks. A “humanized form” of an antibody, e.g., a non-human antibody, refers to an antibody that has undergone humanization.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the VH (H1, H2, H3), and three in the VL (L1, L2, L3).

HVRs include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, MD (1991), NIH Publication 91-3242);
(c) antigen contacts occurring at amino acid residues 27c-36 (L1), 46-55 (L2), 89-96 (L3), 30-35b (H1), 47-58 (H2), and 93-101 (H3) (MacCallum et al., J. Mol. Biol. 262 (1996) 732-745); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “individual” or “subject” is a mammal. Mammals include, but are not limited to, domesticated animals (e.g. cows, sheep, cats, dogs, and horses), primates (e.g., humans and non-human primates such as monkeys), rabbits, and rodents (e.g., mice and rats). In certain preferred embodiments of all aspects and embodiments according to the current invention, the individual or subject is a human.

An “isolated” antibody is one, which has been separated from a component of its natural environment. In some embodiments of all aspects and embodiments according to the current invention, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S., et al., J. Chrom. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-human CD20/human transferrin receptor antibody” refers to one or more nucleic acid molecules encoding the antibody heavy and light chains of the antibody, including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3), whereby between the first and the second constant domain a hinge region is located. Similarly, from N- to C-terminus, each light chain has a variable region (VL), also called a variable light domain or a light chain variable domain, followed by a constant light chain (CL) domain. The light chain of an antibody may be assigned to one of two types, called kappa (κ) and lambda (λ), based on the amino acid sequence of its constant domain.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments of all aspects and embodiments according to the current invention, RG6035 is used to delay development of a disease or to slow the progression of a disease, especially of multiple sclerosis.

The term “variable region” or “variable domain” refers to the domain of an antibody's heavy or light chain that is involved in binding of the antibody to the antigen. The variable domains of the heavy chain and light chain (VH and VL, respectively) of a native antibody generally have similar structures, with each domain comprising four conserved framework regions (FRs) and three hypervariable regions (HVRs) (see, e.g., Kindt, T. J. et al., Kuby Immunology, 6th ed., W.H. Freeman and Co., N.Y. (2007), page 91).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichloro triethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERER, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

### Recombinant Methods and Compositions

Antibodies may be produced using recombinant methods and compositions, e.g., as described in U.S. Pat. No. 4,816,567. Isolated nucleic acid encoding RG6035 can be provided. One or more vectors (e.g., expression vectors) comprising such nucleic acid can be provided. A host cell comprising such nucleic acid can be provided. The host cell can be a eukaryotic, e.g. a Chinese Hamster Ovary (CHO) cell or lymphoid cell (e.g., Y0, NS0, Sp2/0 cell). A method of making RG6035 can be provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of RG6035, one or more nucleic acids encoding the antibody, e.g., as described above, is/are isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for the expression of RG6035 are mammalian cell lines that are adapted to grow in suspension. Useful mammalian host cell lines include Chinese hamster ovary (CHO) cells, including DHFR-CHO cells (Urlaub, G. et al., Proc. Natl. Acad. Sci. USA 77 (1980) 4216-4220). For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

### Multiple Sclerosis

Multiple sclerosis (MS) is a chronic, demyelinating, immune-mediated, inflammatory disease of the central nervous system (CNS). [8a]

Historically, it was believed that CNS tissue damage in MS was mediated by infiltrating proinflammatory CD4+ T-cells [9a, 10a]. However, it is now known that B-cells are also key contributors to MS immunopathology, as demonstrated by the observed clinical efficacy of CD20-targeting, peripherally B-cell-depleting therapies in relapsing-remitting MS [11a-17a]. Recent publications suggest that CNS-compartmentalized B-cells are particularly detrimental in progressive MS (PMS) [18a]. The presence of B-cell-rich lymphoid-like structures in PMS is associated with an unfavorable disease course, including cortical demyelination, brain atrophy, microglia activation, as well as synaptic and neuronal loss [18a, 19a].

Thus, growing evidence strongly suggests involvement of B-cells in the pathophysiology of MS. Whilst several B-cell depleting monoclonal antibody (mAb) therapies are now approved for MS, they are unable to actively cross the blood-brain-barrier and address the disease in the CNS. Thus, one of the key hurdles of the development of novel B-cell depletion monoclonal antibody therapies remains their delivery to the brain.

Although progress has been made in the treatment of MS, delaying disease progression by effectively delivering therapeutics to the CNS remains an unmet medical need. Studies on post-mortem brain tissue of secondary progressive MS (SPMS) patients revealed the presence of B-cell-containing ectopic lymphoid-like structures and the lack of regulatory T-cells in those structures. [7,8,29] This suggests that the latter promote auto-antigen-specific adaptive immune responses that exacerbate chronic disease.[30] Given this evidence, effective depletion of B-cells behind the BBB and blood-CSF-barrier is crucial to halter the sustained inflammation.

Recent scientific evidence suggests that brain-compartmentalized B-cells within lymphoid follicle-like structures of the brain and spinal cord of MS patients are associated with disease progression.[29,31] Besides approved MS therapies with varying mechanisms of action, including T-cell-targeting therapies, recently approved B-cell-targeting therapies have also shown efficacy.[32] The importance of this mode of action has been reinforced with the successful development of B-cell-depleting monoclonal antibodies that target the CD20 surface antigen, such as ocrelizumab and ofatumumab.[33] Even though many drugs that target the pathological inflammatory mechanisms associated with relapses and relapse associated worsening are available, to date, only ocrelizumab is indicated for primary progressive MS (PPMS). Ocrelizumab is a recombinant humanized mAb that selectively targets CD20-expressing B-cells.[15] Increasing evidence supports the hypothesis that CNS compartmentalized inflammation contributes significantly to disability progression.[8,29,31] Thus, therapeutic candidates that target this region are needed to allow for an effective therapy.

The BBB prevents neurotoxic plasma components, blood cells, and pathogens from entering the brain. Simultaneously, the BBB strongly impedes access of systemically administered mAbs to the brain, limiting delivery of the amount of therapeutically active mAbs and achieving therapeutic concentrations. This presents a major challenge in the development of mAb-based therapies for brain disorders.

### The Approach According to the Current Invention

Using a PK/PD study a cynomolgus monkey model was designed to determine the therapeutic effect of RG6035, i.e. to show that RG6035 could effectively access the immune-privileged area of the CNS behind the BBB and deplete B-cell populations.

RG6035 is an engineered trivalent, bispecific monoclonal antibody comprising a variant of obinutuzumab and an additional Fab specific for human TfR1. The additional binding to TfR1 allows an improved transfer of RG6035 across the BBB by TfR1-mediated transcytosis.

Without being bound by this theory, it is assumed that the TfR1-mediated uptake constitutes an important clearance pathway in the periphery and this is believed to be additive to other clearance mechanisms such as the non-specific clearance of the antibody component.[37] This would lead to a higher total clearance of the brain shuttle construct compared with a conventional monoclonal antibody.

The doses applied in the study amounted for 0.13% to 3.4% of serum concentration in the brain. These are higher than those achievable with a typical antibody. Thus, brain penetration was higher for RG6035 than for a normal IgG. That is brain exposure was with a 25-fold higher brain AUC at steady state compared with a typical IgG, such as obinutuzumab.

Multiple CSF sampling time points are needed to generate a full PK profile of a compound in the CNS. Therefore, the cannulation of the cisterna magna was used and compared to lumbar puncture for the in-vivo sampling of cerebrospinal fluid. Indeed, cisterna magna cannulation offers the advantages of repeated sampling without anesthesia-induced bias.

It has been found that concentration over time was similar in both compartments, with the ratio between the concentrations in the cisterna magna and in the lumbar puncture ranging from 0.2 to 1.1, indicating no difference between the two sampling methods. Chronic CSF sampling models by surgical implantation of a catheter into the brain allows repeated sampling of CSF in conscious, freely moving animals.

The ability of obinutuzumab to induce direct B-cell death was identified as the driving mechanism for efficient depletion of immunologically active B-cells in an Fc-region-effector-function-independent manner. Reducing the potency of the Fc-region in RG6035 resulted in less cytokine release and fewer adverse events in human whole blood assays compared with obinutuzumab.[38] A more favorable safety profile, particularly in combination with the TfR1 brain shuttle, is integral of the Fc-silent type II CD20/TfR1 antibody (BS-obinutuzumab-PGLALA) RG6035. It could be shown that despite the combination of an Fc-region-effector-function-silent obinutuzumab with a TfR1 brain shuttle module both functionalities, i.e, the brain shuttle functionality as well as the CD20 binding functionality including the B-cell killing property (receptor binding and direct B-cell depletion), are preserved and that brain exposure is increased in comparison to a standard, non-TfR1-binding IgG antibody.

The increase in exposure was over proportional compared to the increase in dose with dose-normalized Cₘₐₓ and AUC increasing by roughly 4-8-fold as the dose increased from 0.3 mg/kg to 10 mg/kg. Without being bound by this theory, it is assumed that these observations are consistent with TMDD and it is believed that this was related to the TfR1 and/or CD20 binding and subsequent trafficking. It has been found that even at the highest single dose of 10 mg/kg, drug clearance was approximately 3-7-fold higher than expected for similar molecules, such as a standard IgG antibody. Without being bound by this theory, it is assumed that this increased clearance is consistent with TMDD via both TfR and CD20, however with varying contributions to the non-linearity in the dose exposure relationship.

Even though preclinical murine MS models are widely used in this field, their utility for RG6035 is extremely limited. The BBB in these models is leaky and overall B-cell depletion has demonstrated marginal and inconsistent results in murine MS models.[39,40] Furthermore, the difference between the type I and type II anti-CD20 antibody mechanisms for induction of direct B-cell death does not apply in wild-type rodents. It therefore can be expected that B-cell depletion in the CNS of mice has limited value for extrapolating therapeutic effects in humans.[40] Additionally, and perhaps most importantly, the clinical brain shuttle does not bind to rodent, canine or porcine TfR1 homologues or to non-primate CD20. Binding affinities of RG6035 to human and cynomolgus monkey TfR1 have been shown to be similar via surface plasmon resonance (equilibrium dissociation constant KD huTfR1 421±5 nM, KD cyTfR1 694±11 nM at 37° C.). The species selection was, thus, based on the binding affinity restricted to human and cynomolgus monkey. There is also high homology in the constant regions of endogenous primate IgGs in comparison to the human therapeutic antibody, thereby reducing the extent to which ADAs may be formed. Considering these factors together, murine MS models cannot be effectively used for dose prediction in humans of RG6035.

The CNS of cynomolgus monkeys is similar to that of healthy humans and contains only very few, if any, B-cells.[41,42] Therefore, healthy cynomolgus monkeys cannot be used as a model for B-cell depletion in human CNS.

It has now been found that peripheral pharmacodynamics (PD) of B-cells in blood in combination with CNS exposure to RG6035 in cynomolgus monkeys can be used to simulate and predict doses required to achieve therapeutic active levels of RG6035 in the human brain. The depletion of B-cells in lymphoid tissues shows that B-cell depletion is not limited to blood.

Given the qualitative overlap of CD20 and CD19, CD19 has been used as marker for total B-cells, capturing B-cell maturation states from pro-B-cell to plasmablasts.[43] The drastic decrease of CD19+-B-cells in surrogate lymphoid organs (e.g. lymph nodes) upon treatment with RG6035 illustrates their long-lasting and effective depletion. Without being bound by this theory, it is assumed that several mechanisms of excretion are thought to be involved in the clearance of RG6035. Specific clearance of RG6035 bound to the CD20 target on B-cells may be mediated by the destruction of said B-cells. RG6035 may be sustained in the circulation by recycling through the neonatal Fc receptor.

Herein a translational PK/PD model based on PK/PD data on blood B-cell depletion in cynomolgus monkeys was used for the identification of a pharmacologically active dose range for RG6035.

Since no data is available for central (brain) B-cell depletion in any relevant animal model, the current invention is based, at least in part, on deliberate projections that have been made:
(i) the PK can actually be scaled from cynomolgus monkeys;
(ii) the brain uptake of RG6035 is correctly estimated in cynomolgus monkeys and translates to humans;
(iii) the potency of RG6035 in brain is similar to the one in the periphery, as it is independent of effector cell or complement presence;
(iv) the potency of B-cell depletion can be translated to humans using the ratio of ex-vivo-measured potencies in human and cynomolgus monkey blood (˜4-time higher potency in human than in cynomolgus monkey).

With this translational PK/PD model it was possible to estimate the pharmacologically active RG6035 systemic exposure for therapeutic efficacy, defined herein as ≥60%, preferably ≥95%, B-cell depletion in the brain (as assessed in CSF).

It has been found based on this assessment, that short dosing intervals (e.g. once every week, once every two weeks or once every four weeks) are advantageous for maintenance of B-cell depletion in the CSF.

Despite the small sample size (n=2 per group) used in this study for some of the PK analysis, due to ethical constraints associated with the cynomolgus monkey model, it was possible to evaluate the capacity of RG6035 to cross the BBB and deplete B-cells in blood and secondary lymphoid tissues which enabled the identification of an efficacious human dose.

It is expected that with a dose of 70 mg once every week for four weeks about 60% B-cell depletion can be achieved, with a dose of 200 mg once every week for four weeks 80-90% B-cell depletion can be achieved and with a dose of 700 mg once every week for four weeks more than 95% B-cell depletion can be achieved. This is shown in FIG. 1.

### Brain Shuttle—CD20 Construct

CD20 is a protein expressed on the surface of B-cells (from pro-B-cells to plasmablasts where it is expressed at low levels) and is a clinically validated therapeutic target for MS [20a]. There are two classes of anti-CD20 antibodies: Type I antibodies such as rituximab, ocrelizumab or ofatumumab, and Type II antibodies like tositumomab or obinutuzumab. [22a, 23a]. Obinutuzumab is differentiated from rituximab and type I antibodies by virtue of mediating stronger direct cell death induction independent of Fc-region-effector-functions, and enhanced ADCC/ADCP due to Fc-engineering, but reduced CDC [48].

In order to efficiently target CNS-compartmentalized B-cells in progressive MS, a CD20 mAb would need to possess properties for extravascular B-cell depletion and the ability to cross the BBB. Without being bound by this theory, it is reasoned that the Type II CD20 antibody obinutuzumab is particularly suited to destroy brain resident B-cells along with properties of efficient extravascular B-cell depletion due to its advantageous B-cell depletion potential, independent of effector functions (ADCC/CDC), that are limited in the CNS. Obinutuzumab not only mediates Fc-region-effector-function-dependent mechanisms but more importantly also induces Fc-region-effector-function-independent, non-apoptotic B-cell death upon CD20 receptor binding [22a, 23a]. RG6035 is a fusion between obinutuzumab and a brain shuttle module, wherein the Fc-region of obinutuzumab was made Fc-region-effector-function-silent by introducing the P329G/L234A/L235A mutation (PGLALA mutation). This improves the safety whilst retaining B-cell depleting properties.[24a] Importantly, obinutuzumab containing the PGLALA mutation is still able to deplete B-cells in whole blood and to mediate anti-tumor efficacy in xenograft models in contrast to Fc-region-effector-function-silenced version of other anti-CD20 antibodies that lost B-cell depletion activity in absence of Fc-region-effector-functions.

Thus, RG6035 has an advantageous safety profile compared with an Fc-region-effector-function-competent construct, while maintaining the ability to cross the BBB and to deplete B-cells.

In more detail, RG6035 is a trivalent, bispecific antibody comprising
a) one full-length antibody comprising two pairs each of a full-length antibody light chain and a full-length antibody heavy chain, wherein the binding sites formed by each of the pairs of the full-length heavy chain and the full-length light chain specifically bind to human CD20, and
b) one additional Fab that is fused to the C-terminus of one heavy chain of the full-length antibody of a), wherein the binding site of the additional Fab specifically binds to human transferrin receptor 1,
wherein each of the full-length antibody light chains comprises in the constant light chain domain at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein each of the full-length antibody heavy chains comprises in the first constant heavy chain domain at position 147 an glutamic acid residue (instead of the wild-type lysine residue; K147E mutation) and at position 213 an glutamic acid residue (instead of the wild-type lysine amino acid residue; K213E mutation) (numbering according to Kabat),
wherein the additional Fab specifically binding to human transferrin receptor 1 comprises a domain crossover such that the constant light chain domain and the constant heavy chain domain 1 are replaced by each other.

Thus, RG6035 is composed of four polypeptides that have the amino acid sequence of SEQ ID NO: 01, SEQ ID NO: 02, SEQ ID NO: 03 and SEQ ID NO: 05.

In one embodiment of all aspects and embodiments according to the current invention, RG6035 is a trivalent, bispecific antibody comprising two polypeptides that comprise the amino acid sequence of SEQ ID NO: 01, one polypeptide that comprises the amino acid sequence of SEQ ID NO: 02, optionally with additional N-terminal glutamine (Q) or pyroglutamic acid (pE) residue or/and optionally with additional C-terminal lysine (K) amino acid residue, one polypeptide that comprises the amino acid sequence of SEQ ID NO: 03, and one polypeptide that comprising the amino acid sequences of SEQ ID NO: 04, optionally with additional N-terminal glutamine (Q) or pyroglutamic acid (pE) residue.

In one preferred embodiment of all aspects and embodiments according to the current invention, RG6035 is a trivalent, bispecific antibody comprising two polypeptides that comprise the amino acid sequence of SEQ ID NO: 01, one polypeptide that comprises the amino acid sequence of SEQ ID NO: 02, optionally with additional N-terminal glutamine (Q) or pyroglutamic acid (pE) residue and/or optionally with additional C-terminal lysine (K) amino acid residue, one polypeptide that comprises the amino acid sequence of SEQ ID NO: 03, and one polypeptide that comprising the amino acid sequences of SEQ ID NO: 05, optionally with additional N-terminal glutamine (Q) or pyroglutamic acid (pE) residue.

SEQ ID NO: 01 has the amino acid sequence:
DIVMTQTPLSLPVTPGEPASISCRSSKSLLHSNGITYLYWYLQKPGQS
PQLLIYQMSNLVSGVPDRFSGSGSGTDFTLKISRVEAEDVGVYYCAQN
LELPYTFGGGTKVEIKRTVAAPSVFIFPPSDRKLKSGTASVVCLLNNF
YPREAKVQWKVDNALQSGNSQESVTEQDSKDSTYSLSSTLTLSKAD
YEKHKVYACEVTHQGLSSPVTKSFNRGEC.
SEQ ID NO: 02 has the amino acid sequence:
VQLVQSGAEVKKPGSSVKVSCKASGYAFSYSWINWVRQAPGQGLE
WMGRIFPGDGDTDYNGKFKGRVTITADKSTSTAYMELSSLRSEDTAV
YYCARNVFDGYWLVYWGQGTLVTVSSASTKGPSVFPLAPSSKSTSG
GTAALGCLVEDYFPEPVTVSWNSGALTSGVHTFPAVLQSSGLYSLSS
VVTVPSSSLGTQTYICNVNHKPSNTKVDEKVEPKSCDKTHTCPPCPAP
EAAGGPSVFLFPPKPKDTLMISRTPEVTCVVVDVSHEDPEVKFNWYV
DGVEVHNAKTKPREEQYNSTYRVVSVLTVLHQDWLNGKEYKCKVS
NKALGAPIEKTISKAKGQPREPQVCTLPPSRDELTKNQVSLSCAVKGF
YPSDIAVEWESNGQPENNYKTTPPVLDSDGSFFLVSKLTVDKSRWQQ
GNVFSCSVMHEALHNHYTQKSLSLSPG.
SEQ ID NO: 03 has the amino acid sequence:
AIQLTQSPSSLSASVGDRVTITCRASQSISSYLAWYQQKPGKAPKLLI
YRASTLASGVPSRFSGSGSGTDFTLTISSLQPEDFATYYCQQNYASSN
VDNTFGGGTKVEIKSSASTKGPSVFPLAPSSKSTSGGTAALGCLVKDY
FPEPVTVSWNSGALTSGVHTFPAVLQSSGLYSLSSVVTVPSSSLGTQT
YICNVNHKPSNTKVDKKVEPKSC.
SEQ ID NO: 04 has the amino acid sequence:
SMQESGPGLVKPSQTLSLTCTVSGFSLSSYAMSWIRQHPGKGLEWIG
YIWSGGSTDYASWAKSRVTISKTSTTVSLKLSSVTAADTAVYYCARR
YGTSYPDYGDASGFDPWGQGTLVTVSSASVAAPSVFIFPPSDEQLKSG
TASVVCLLNNFYPREAKVQWKVDNALQSGNSQESVTEQDSKDSTYS
LSSTLTLSKADYEKHKVYACEVTHQGLSSPVTKSFNRGEC.
SEQ ID NO: 05 has the amino acid sequence:
VQLVQSGAEVKKPGSSVKVSCKASGYAFSYSWINWVRQAPGQGLE
WMGRIFPGDGDTDYNGKFKGRVTITADKSTSTAYMELSSLRSEDTAV
YYCARNVFDGYWLVYWGQGTLVTVSSASTKGPSVFPLAPSSKSTSG
GTAALGCLVEDYFPEPVTVSWNSGALTSGVHTFPAVLQSSGLYSLSS
VVTVPSSSLGTQTYICNVNHKPSNTKVDEKVEPKSCDKTHTCPPCPAP
EAAGGPSVFLFPPKPKDTLMISRTPEVTCVVVDVSHEDPEVKFNWYV
DGVEVHNAKTKPREEQYNSTYRVVSVLTVLHQDWLNGKEYKCKVS
NKALGAPIEKTISKAKGQPREPQVYTLPPCRDELTKNQVSLWCLVKG
FYPSDIAVEWESNGQPENNYKTTPPVLDSDGSFFLYSKLTVDKSRWQ
QGNVFSCSVMHEALHNHYTQKSLSLSPGGGSGGGGSGGGGSGGGGS
QSMQESGPGLVKPSQTLSLTCTVSGFSLSSYAMSWIRQHPGKGLEWI
GYIWSGGSTDYASWAKSRVTISKTSTTVSLKLSSVTAADTAVYYCAR
RYGTSYPDYGDASGFDPWGQGTLVTVSSASVAAPSVFIFPPSDEQLKS
GTASVVCLLNNFYPREAKVQWKVDNALQSGNSQESVTEQDSKDSTY
SLSSTLTLSKADYEKHKVYACEVTHQGLSSPVTKSFNRGEC.

In one embodiment of all aspects and embodiments of the current invention, RG6035 is a monoclonal trivalent, bispecific antibody.

In CD20 binding assays, the Fc-region-effector-function-competent variant of RG6035 and RG6035 itself demonstrated comparable CD20 binding in a concentration-dependent manner. The brain shuttle constructs had a slightly higher affinity than the corresponding non-brain shuttle control antibodies, i.e. obinutuzumab and obinutuzumab-PGLALA. Without being bound by this theory, it is assumed that this may be caused by additional binding of the brain shuttle moiety to TfR1 expressed on the surface of B-cells.

Maximum B-cell depletion in human CSF spiked with human peripheral blood mononuclear cells (PBMCs) was similar for both RG6035 and obinutuzumab-PGLALA and there were no significant differences between the two constructs in terms of binding and B-cell depletion in ex vivo human tonsil-derived cells, confirming the ability of RG6035 to deplete B-cells stemming from lymphoid tissues.

In more detail, to assess B-cell death induction properties in cerebrospinal fluid (CSF), human peripheral blood mononuclear cells (PBMCs) were incubated with RG6035 and obinutuzumab-PGLALA in CSF from healthy human donors. The average maximum depletion (±standard deviation [±SD]) after 22 hours incubation was 75.11%±5.94 and 69.87%±13.3 for RG6035 and obinutuzumab-PGLALA, respectively. These results indicate strong direct B-cell depleting potency in the CSF cell culture matrix, confirming the susceptibility of B-cells in CSF as an example of central nervous system (CNS)-compartmentalized B-cells to the caspase-independent (non-apoptotic) B-cell depletion mechanism, mediated by RG6035 and obinutuzumab-PGLALA.[15]

This PBMC-CSF assay shows that the mechanism of action of RG6035 and obinutuzumab-PGLALA is an Fc-region-effector-function-independent, direct B-cell killing mechanism leading to efficient B-cell depletion in the CSF. Furthermore, B-cell depletion does not require availability of the complement system.

RG6035 binding and B-cell depletion were also assessed in an ex vivo autologous B-cell depletion assay in human tonsil-derived cells to assess efficacy of direct cell death induction. Without being bound by this theory, it is assumed that B-cells from tonsil tissue may better reflect the response of B-cells in secondary lymphoid tissues compared with peripheral blood B-cells.

The assessment indicated that human tonsils contain a large proportion of B-cells and substantial heterogeneity of B-cell subpopulations, particularly naive B-cells, tissue resident memory B-cells, and immunologically active germinal center (GC) B-cells.

It has been found that obinutuzumab-PGLALA induced direct CD19+-B-cell death and depletion with a half-maximal effective concentration (EC50) of 0.523±0.134 nM. The ability for direct cytotoxicity was preserved in RG6035 (EC50=0.396±0.177 nM). A cytotoxicity dose effect depending on the antibody concentration was observed 22 hours after the addition of both constructs to tonsil-derived cells and concentration dependent CD19+-B-cell depletion for three tonsil donors was shown. Maximum depletion was reached for both compounds in a concentration range between 25 nM and 100 nM (see Table 1). There was no significant difference in maximum B-cell depletion between the two constructs at any of the tested concentrations. RG6035 was able to reduce the amount of CD19+ human tonsil B-cells in this experiment by a maximum mean of 60%.

TABLE 1
Obinutuzumab-PGLALA and RG6035 binding and
B-cell depletion in an ex vivo autologous
human tonsil-derived B-cell depletion assay.

| | Obinutuzumab- | |
|---|---|---|
| nM | PGLALA | RG6035 |
| T1 | 0.44 | 0.44 |
| T2 | 0.46 | 0.20 |
| T3 | 0.68 | 0.55 |
| average | 0.52 | 0.40 |
| standard- | 0.13 | 0.18 |
| deviation | | |

Three tonsil donors were used to determine the EC50 value for CD19+-B-cell depletion as well as B-cell subset depletion, It has been found that there was a different cellular depletion profile between the three donors. A comparative analysis showed a correlation between maximal B-cell subset depletion and surface CD20 expression, with the highest CD20 expression on transitional and the lowest CD20 expression on naive B-cells. The highest maximal depletion was observed on transitional and the lowest on naive B-cells. B-cells with intermediate CD20 expression levels showed also intermediate maximal B-cell depletion levels. Thus, the response to RG6035 correlates with the degree of CD20 surface expression.

Thus, RG6035 is able to induce direct cell death and deplete extravascular human B-cell subsets located in secondary lymphoid tissues.

Without being bound by this theory, it is assumed that an explanation for incomplete depletion of B-cells could be the differential expression of CD19 and CD20 on late-stage B-cells, specifically plasmablasts, which express low levels of CD20 and plasma cells, which express no CD20, while both cell types express CD19. Furthermore, a potential modulation of CD19 expression levels after CD20 antibody incubation could explain insufficient detection of maximal depletion [Jones, D. J., et al., Arthritis Rheum. 64 (2012) 3111-3118].

Thus, the presence of a TfR1 binding moiety does not negatively interfere with the B-cell-depleting potency. No significant differences in EC50 and maximal depletion were observed between the two constructs, proving for a preserved direct B-cell death induction ability of RG6035 despite BS-mediated TfR1 co-binding.

RG6035 and BS-obinutuzumab displayed a 30-fold higher intracellular uptake in Madin-Darby canine kidney II (MDCKII) cells transfected with huTfR1 (MDCKII-huTfR) following a 30-minute loading (pulse) phase compared with huTfR1-negative non-transfected cells (MDCKII-parental). Following huTfR1-mediated uptake, comparable transcytosis activity profiles were observed for both brain shuttle constructs, with the amount of IgG released into the extracellular compartment being identical. The non-target binding huTfR1 positive control molecule, BS-DP47-PGLALA showed similar huTfR1-mediated uptake and transcytosis. Data are the mean of triplicates+/−SD. Following a loading (pulse) phase of 30 minutes, cells were extracted and analyzed for intracellular IgG content using ELISA. The results are expressed as uptake of IgG normalized against the total cell protein in each well (ng IgG/mg). The results are shown in FIGS. 2-4.

The transcytosis efficiency is shown in FIG. 5. The ng IgG transcytosed are compared with the ng IgG in the corresponding intracellular compartment at the beginning (t=0) of the chase (expressed as a %).

Thus, Fc-effector-function-silencing and the conjugation to a brain shuttle module do not impair CD20 binding nor TfR1-mediated transcytosis. The extent of direct B-cell death as a result of binding was comparable and concentration-dependent for these molecules.

Human CD20 transgenic mice were treated via intravenous (IV) administration with a surrogate antibody consisting of the human CD20 binder (obinutuzumab variable domains) and a murine TfR binding brain shuttle module (mBS-obinutuzumab) at 0.6 mg/kg, 1.3 mg/kg, and 13.3 mg/kg, or with obinutuzumab (non-shuttled) at 0.5 mg/kg, 1 mg/kg, and 10 mg/kg (equimolar concentrations). Similar blood B-cell depletion was achieved with both constructs on Day 6, without evidence of a dose-dependent response. A higher degree of B-cell depletion with obinutuzumab compared with mBS-obinutuzumab antibody was observed. When comparing effects in lymphoid tissues, B-cell killing by mBS-obinutuzumab was greater than by mBS-obinutuzumab-PGLALA. The effect was more pronounced in the spleen (p<0.05 at all doses) than in the lymph node (p<0.05 in 0.6 mg/kg dose group only) as assessed using two-tailed paired t-test. However, in the lymph node, a difference between Fc-region-effector-function-silent and Fc-region-effector-function-competent constructs was only observed in animals that received the lowest dose, showing that comparable B-cell depletion can be achieved with an ADCC-independent molecule.

The results are shown in Table 2.

TABLE 2
Kinetics of B-cell depletion in blood, spleen, and lymph node:
kinetics of B-cell depletion in huCD20 murine blood following
administration of mBS-obinutuzumab or mBS-obinutuzumab-PGLALA
(A) and parental (non-shuttled) antibody (B); data points represent
mean ± SD of N = 6 mice; kinetics of B-cell depletion
of mBS-huCD20 mAbs at Day 6 in the spleen (C) and lymph nodes
(D) of huCD20 and naive mice; differences in the means of B-
cell depletion in the lymph nodes and spleen in mBS-huCD20-WT
and mBS-huCD20-PGLALA-treated vs. naive mice were assessed;
P-values (one-way ANOVA) at all dose groups (0.6, 1.3 and 13.3
mg/kg) were <0.05 in spleen and in lymph nodes.
B220+-cells/% of total leukocytes in blood

| | | dose | | |
|---|---|---|---|---|
| antibody | time | 0.6 mg/kg | 1.3 mg/kg | 13.3 mg/kg |
| mBS- | −1 | 26.33 | 29.53 | 28.25 |
| obinutuzumab | 2 | 18.50 | 23.53 | 22.67 |
| | 6 | 5.63 | 6.71 | 5.31 |
| mBS- | −1 | 24.68 | 24.03 | 23.53 |
| obinutuzumab- | 2 | 37.88 | 33.70 | 30.83 |
| PGLALA | 6 | 13.48 | 12.57 | 14.47 |

B220+-cells/% of total leukocytes in blood

| | | dose | | |
|---|---|---|---|---|
| antibody | time | 0.5 mg/kg | 1.0 mg/kg | 10 mg/kg |
| obinutuzumab | −1 | 42.27 | 47.75 | 45.20 |
| | 2 | 4.11 | 3.87 | 2.31 |
| | 6 | 1.41 | 1.98 | 1.38 |
| obinutuzumab- | −1 | 51.73 | 50.92 | 53.60 |
| PGLALA | 2 | 58.07 | 55.32 | 55.58 |
| | 6 | 31.05 | 26.30 | 35.28 |

| | | dose | | |
|---|---|---|---|---|
| antibody | time | 0.6 mg/kg | 1.3 mg/kg | 13.3 mg/kg |

B220+-cells/% of total leukocytes in spleen (day 6)

| | | | | |
|---|---|---|---|---|
| mBS- | 6 | 18.62 | 17.72 | 4.29 |
| obinutuzumab | | | | |
| mBS- | 6 | 44.47 | 35.38 | 27.25 |
| obinutuzumab- | | | | |
| PGLALA | | | | |

B220+-cells/% of total leukocytes in lymph node (day 6)

| | | | | |
|---|---|---|---|---|
| mBS- | 6 | 6.23 | 7.22 | 4.91 |
| obinutuzumab | | | | |
| mBS- | 6 | 11.95 | 7.45 | 5.46 |
| obinutuzumab- | | | | |
| PGLALA | | | | |

A potential concern regarding Fc-region-effector-function-competent molecules is the induction of infusion-related reactions (IRRs), which involves cross-linking of TfR1 on peripheral cells with Fc gamma receptors on immune cells and their subsequent activation. It has been shown previously that BS-antibody fusion constructs with full effector function can be transported in “stealth mode” in the periphery due to steric hindrance, whilst retaining full activity when engaged with their CNS target in preclinical in vitro and mouse models [27a].

To confirm the absence of a risk of IRRs related to Fc-region-effector-function and the presence of the BS module an in vitro human whole blood assay (WBA) was performed. Additionally, the compounds were administered to huCD20 and huCD20xHIGR3 mice and cytokine release was determined in vitro and temperature change was determined in vivo.

In more detail, in the human WBA no relevant release of any investigated cytokine was observed for obinutuzumab-PGLALA and RG6035 within the concentration range examined (0.01 nM to 1000 nM). In contrast, moderate interferon-gamma (IFN-γ), tumor necrosis factor-alpha (TNF-α), interleukin-6 (IL-6), IL-8, and IL-1β release was observed for obinutuzumab. Moderate-to-strong IL-6 and strong IL-8 cytokine release was observed for BS-obinutuzumab at the highest concentration. All tested constructs demonstrated concentration-dependent B-cell depletion. The comparable BS-obinutuzumab and obinutuzumab B-cell depletion potency in the WBA indicates that the BS moiety has no impact on B-cell killing function.

mBS-obinutuzumab, mBS-obinutuzumab-PGLALA, and obinutuzumab were assessed in a single IV dose study in both huCD20 and huCD20xHIGR3 mice (n=5 animals/group for each compound and mouse model). Slight-to-moderate hypoactivity was recorded 15 minutes after administration of 10 mg/kg mBS-obinutuzumab in all huCD20 and huCD20xHIGR3 mice. However, within two hours after administration, all animals recovered and exhibited normal behavior. Body temperature data demonstrates a clear differentiation between treatments; all animals treated with mBS-obinutuzumab experienced an acute temperature drop of approximately 2-5° C., indicative of an acute IRR, followed by a quick recovery, while animals treated with obinutuzumab or mBS-obinutuzumab-PGLALA experienced no drop in body temperature. Cytokine levels in these groups indicated a typical pattern for classical IRR [29a]; notably, keratinocyte-derived cytokine (KC/CXCL1), monocyte chemoattractant protein-1 (MCP-1), granulocyte colony stimulating factor (G-CSF), and macrophage inflammatory protein-1ß (MIP-1) serum levels were elevated.

Compared to mBS-obinutuzumab-PGLALA, mBS-obinutuzumab induced strong serum release of KC, G-CSF, MIP-1β, MCP-1 and a moderate IL-6 release. Other cytokines remained largely unaffected, with values for TNF-α, IL-2, and IFN-γ were below the limit of detection (2.3 μg/mL, 1.0 μg/mL, and 1.1 μg/mL, respectively). In mice treated with mBS-obinutuzumab-PGLALA, cytokine levels for KC, G-CSF, MIP-1β, and MCP-1, MIP-2 remained largely unaffected or declined moderately.

The maximal tolerated dose (MTD) after a single IV administration was ≥10 mg/kg for both mBS-obinutuzumab and mBS-obinutuzumab-PGLALA.

mBS-obinutuzumab and mBS-obinutuzumab-PGLALA variants were administered as a single IV dose (0.6 mg/kg, 1.3 mg/kg, or 13.3 mg/kg) to female huCD20xHIGR3 mice. Following IV administration, all mice survived until scheduled sacrifice on Day 3 (N=5), Day 8 (N=5), or Day 22 (N=5) post administration. No clinical signs or effects on body weight were observed. IL-6 levels were below the lower limit of quantification (1.94 μg/mL) in all animals on all investigated days (Days 3, 8, and 22). Animals treated with mBS-obinutuzumab (13.3 mg/kg) demonstrated severe decreases in reticulocyte count and immature reticulocyte fraction on Day 3, consistent with an inhibition/depletion of bone marrow red blood cell (RBC) precursors. In a single animal treated with mBS-obinutuzumab (13.3 mg/kg), the neutrophil count was increased on Day 3, consistent with an acute phase response. Slightly decreased white blood cells (WBCs) and lymphocyte counts on Day 8 were consistent with expected pharmacology of B-cell killing. In contrast, only a slight decrease in mean corpuscular volume was observed on Day 8 in animals treated with mBS-obinutuzumab-PGLALA.

In blood, mBS-obinutuzumab-PGLALA increased B-cell numbers on Day 3 followed by depletion from Day 8 up to Day 22 at all three dose levels investigated. The most pronounced B-cell depletion (˜25%) was observed with the 1.3 mg/kg dose on Day 22. Treatment with mBS-obinutuzumab led to ˜20% B-cell depletion on Day 3, reaching a maximum of ˜50% depletion on Day 22 at the 13.3 mg/kg dose. On Day 8, treatment at 0.6 mg/kg and 1.3 mg/kg led to a similar magnitude of B-cell depletion (10-20%), whereas a more pronounced depletion effect of mBS-obinutuzumab was observed at 13.3 mg/kg. The results are shown in FIGS. 6 to 8.

No effect on spleen and inguinal lymph node weight was recorded with either of the investigated constructs. In one animal treated with mBS-obinutuzumab, gross findings consisted of an increase in spleen size at 13.3 mg/kg on Day 3, which correlated histologically with an increase in extramedullary hematopoiesis. There were no gross findings in inguinal lymph nodes or sternal bone marrow following treatment with either mBS-obinutuzumab antibody.

In inguinal lymph nodes, the most severe reduction of B-cell numbers (˜50%) was observed on Day 3. Without being bound by this theory, it is assumed that this may not necessarily reflect B-cell depletion, but rather egress to peripheral blood. The results are shown in FIGS. 9 to 11.

In the spleen, no effect of mBS-obinutuzumab-PGLALA was observed and only a minimal and non-dose-dependent effect was observed with mBS-obinutuzumab. Histological findings in the spleen consisted of an increase in extramedullary hematopoiesis on Days 3 and 8 with return to normal on Day 22 with both mBS-obinutuzumab constructs. There was no difference in the extent of hemosiderosis (evidence of iron accumulation) in the spleen between treated and control animals. There were no histological findings in inguinal lymph nodes and bone marrow. The results are shown in FIGS. 12 to 14.

Following single-dose IV administration of 13.3 mg/kg of the mBS-obinutuzumab antibodies, pharmacokinetic (PK) parameters in serum were calculated based on composite concentration-time profiles. PK parameters were similar between both constructs. Following injection of a blood tracer and perfusion, both mBS-obinutuzumab constructs were detected in brain tissue at 24, 48, and 168 hours post administration, except for one animal treated with mBS-obinutuzumab at 168 hours, where the brain concentration was below the limit of quantification. Brain-to-serum ratios ranged between 0.0183 and 0.131 for mBS-obinutuzumab-PGLALA and between 0.00951 and 0.153 for mBS-obinutuzumab, whereby for the latter blood contamination in brain was negligible.

TABLE 3
PK parameters of the murine BS constructs.

| | huCD20 transgenic mice | |
|---|---|---|
| | | mBS-obinutuzumab- |
| PK parameter | mBS-obinutuzumab | PGLALA |
| Clearance [mL/min/kg] | 0.042 | 0.048 |
| Volume of Distribution [L/kg] | 0.089 | 0.098 |
| Half-life [h] | 45.9 | 42.0 |

Immunohistochemistry for human IgG, which detects the blood tracer and both mBS-obinutuzumab constructs, demonstrated a comparable staining distribution and intensity in mice treated with the two mBS-obinutuzumab constructs with different parenchymal staining intensity at each time point, which was consistent with the brain PK profiles. The strongest diffuse brain parenchymal and meningeal vessel staining was at 24 hours post administration, with only vascular endothelial cell staining at the 168-hour time point, most likely related to the presence of blood tracer.

The cynomolgus monkey is the only cross-reactive species for both the CD20 and TfR1 binding moieties of RG6035. PK, pharmacodynamic (PD) and tolerability studies in cynomolgus monkeys were performed to assess the PK/PD and safety profile of RG6035 following single-dose IV administration.

Two separate studies were conducted, from which four animals per group were compared. Animals were treated with a single IV slow bolus of 10 mg/kg RG6035 or vehicle control with a 15-day observation period followed by euthanasia and necropsy, or 10 mg/kg BS-obinutuzumab with an 8-week observation period. Evaluated in-life parameters included clinical observations, body weight, body temperature, clinical pathology (hematology, clinical chemistry, and coagulation), immunophenotyping, cytokine evaluation, macroscopic and histological examination.

Following single-dose IV administration of 10 mg/kg of the constructs, PK parameters in cynomolgus monkey serum were similar. The concentration of RG6035 in CSF was also evaluated and showed good brain penetration, with an area under the concentration-time curve from 0 to 168 hours of 242 nmol*h/L based on composite profile.

TABLE 4
PK parameters of the constructs in cynomolgus monkey.

| | cynomolgus monkey | |
|---|---|---|
| | | BS-obinutuzumab- |
| PK parameter, mean (CV %) | BS-obinutuzumab | PGLALA |
| Clearance [mL/min/kg] | 0.87 (21.2) | 1.33 (28.6) |
| Volume of Distribution [L/kg] | 0.031 (4.9) | 0.0423 (13.0) |
| Half-life [h] | 24.0 (16.6) | 35.3 (26.1) |

RG6035 was well tolerated in all animals and there were no treatment-related changes in clinical observations, body weight, body weight development, or body temperature. There were also no safety-relevant effects on blood cytokine levels, clinical chemistry (including transferrin, iron, ferritin, unsaturated iron binding capacity, and total iron binding capacity), blood soluble transferrin receptor, and macroscopic or histopathological features.

There was no evidence to suggest RG6035 at a single dose of 10 mg/kg interferes with erythropoiesis.

Minimal to mild and transient clinical pathology changes in animals treated with RG6035 were limited to decreases in the content of hemoglobin in reticulocytes and generally non-dose-related increases in C-Reactive Protein, both changes seen as early as Day 2-3 and/or 4. Transient, minimal to moderate decreases in red cell mass parameters (RBC count, hemoglobin and hematocrit) and increases in reticulocyte counts were observed as early as Day 2-8, with a nadir generally apparent on Day 3/4, in all animals including the controls. These changes were attributed to the multiple blood collections. To assess the functional state of bone marrow erythroblasts, blood samples were taken at pre-dose, and at Days 1, 2, 3, 4, 6, 8 and 44 and the serum subjected to determine the hepcidin and erythropoietin (EPO) concentrations. Mildly-to-moderately elevated EPO serum concentrations and a clear trend for suppression of hepcidin were noted in all dose groups, including controls. These findings are indicative of normal regenerative erythropoiesis in response to multiple blood collections, consistent with the observed minimal-to-moderate drop in red cell mass parameters (RBC count, hemoglobin, and hematocrit levels) and the increase in reticulocyte counts. The decrease in the content of hemoglobin in reticulocytes might indicate a concurrent early iron restriction due to the binding of RG6035 to TfR1.

Based on these findings, there is no evidence that RG6035 at a single dose of 10 mg/kg interferes at a relevant level with normal regenerative erythropoiesis.

BS-obinutuzumab was generally well tolerated in the cynomolgus monkey, except in one animal, which exhibited transient clinical signs of subdued and/or decreased activity on Days 2-3 post dosing. Overall, although there were some post-treatment changes in cytokine levels in some animals, none appeared to be proportional to the administered dose. BS-obinutuzumab administration at 10 mg/kg induced transient changes in hematology, coagulation, and clinical chemistry parameters consistent with repeated blood collection and acute phase response.

The expected PD effect of lower lymphocyte numbers for both constructs was observed microscopically in mesenteric lymph nodes and spleen of some of the assessed animals, along with decreased peripheral blood CD19+-B-cells, and CD86-activated B-cell counts starting at 4 hours after dosing until Day 15, CD3+-T-cell and CD16+-NK-cell counts starting at 4 hours after dosing and recovering from Day 4, and an increase in IL-6 levels at the 1- and 4-hour time points post dosing, and a slight increase in MCP-1 levels at 4 hours post dosing only. Without being bound by this theory, it is assumed that this is related to the mode of action of RG6035.

In both cynomolgus monkey studies, there was rapid decrease in blood B-cell numbers following administration. The maximum mean reduction in B-cell count at 4 hours post dosing was approximately 89% for BS-obinutuzumab and 83% for RG6035. A maximum decrease in T-cell count (29%) was observed with both constructs at 4 hours post dosing; this decrease was transient, with a return to baseline levels after 24-96 hours. The maximum decrease for NK-cells (43%) was also observed 4 hours post dosing for both constructs; this decrease was also transient, with a return to baseline levels after 96-168 hours. Almost no change was observed for monocyte levels post dosing. All animals developed ADAs, independent of the Fc-region-effector-function status of the constructs. ADAs in cynomolgus monkeys are considered to be not predictive of immunogenicity in humans [29a].

The results are shown in FIG. 15 and Table 5.

TABLE 5
B-cell depletion following antibody administration in cynomolgus
monkeys; B-cell numbers following antibody administration are
presented as a percentage of B-cells measured at baseline (100%);
data represents mean measurements from N = 4 animals ± SD.

| time | % of baseline | |
|---|---|---|
| [h] | RG6035 | BS-obinutuzumab |

| | | | | |
|---|---|---|---|---|
| 4 | 17 | ±2 | 6 | ±2 |
| 24 | 27 | ±3 | 15 | ±3 |
| 72 | 34 | ±8 | 7 | ±3 |
| 168 | 17 | ±8 | 13 | ±4 |
| 336 | 57 | ±6 | 15 | ±4 |

In cynomolgus monkeys treated with RG6035 on Day 2, there were mild-to-marked increases in absolute neutrophil counts and monocyte counts reflected in increases in total white blood cell counts. Minimal decreases in red blood cell (RBC) mass parameters were noted on Day 8, which were accompanied by moderate increases in absolute reticulocyte counts, indicating increased erythropoiesis secondary to repeated blood collection. Clinical chemistry changes consisted of mildly decreased phosphorus concentrations on Day 2, and minimal-to-mild decreased triglyceride and cholesterol concentrations on Day 2 and/or Day 8. Additionally, minimal increases in fibrinogen, C-reactive protein, haptoglobin, and ferritin concentrations, and minimal decreases in iron concentrations were observed on Day 2 and/or Day 8. Together with the changes in absolute neutrophil and monocyte concentrations, these changes were indicative of an acute phase response. On Day 49, the observed changes in hematology, coagulation, and clinical chemistry (with the exception of triglyceride changes at 1 mg/kg) were no longer present, indicating recovery. These findings are indicative of normal regenerative erythropoiesis in response to multiple blood collections, consistent with the observed minimal-to-moderate drop in red cell mass parameters (RBC count, hemoglobin, and hematocrit levels) and the increase in reticulocyte counts. Based on this, there is no evidence that RG6035 at a single dose of 10 mg/kg interferes with normal regenerative erythropoiesis.

In summary, it has been shown that RG6035 is well tolerated, with no indication of risk for IRRs, while retaining B-cell killing properties. It has been demonstrated that there is a TfR1-dependent uptake of RG6035 into the brain in huCD20 transgenic mice. Taken together, RG6035 is able to and has advantageous properties to target and deplete CNS-located B-cells in MS more effectively, as compared to currently available therapies. It has been shown that for peripherally expressed targets, Fc-region-effector-function-silencing mitigates the risk of IRRs and effects on reticulocytes, resulting in an advantageous safety profile.

RG6035 (RO7121932) is currently in clinical trials in human MS patients (NCT05704361).

### Some Specific Embodiments of the Invention

The current invention is based, at least in part, on the use of a specific brain shuttle-CD20 construct RG6035 (BS-CD20, RO7121932) for the depletion of brain-located B-cells. RG6035 used in the methods of the current invention is a bispecific modular fusion protein of a human transferrin receptor 1 (TfR1)-directed brain shuttle (a TfR1-binding antigen-binding fragment (Fab)) and a paratope of the anti-CD20 antibody, obinutuzumab. TfR1 is abundant at the BBB and is a target for enabling receptor-mediated transport of large molecules across the BBB, while obinutuzumab has a unique mechanism of action: inducing Fc-effector-independent, non-apoptotic, direct B-cell death upon binding of surface-exposed CD20.[14-21] RG6035 has the function to (fully) deplete brain-located B-cells, following repeated administration.

The current invention is based, at least in part, on the finding that a PK/PD model for RG6035 based on the pathophysiology of MS and data observed in blood as a surrogate can be used to model the human situation.

### Therapeutically Effective Amount

The methods of the present invention comprise administering to a subject a composition comprising a therapeutically effective amount of RG6035. As used herein, the term a “therapeutically effective amount” refers to an amount of a compound or pharmaceutical composition sufficient to produce a desired therapeutic effect.

RG6035-treated animals showed a drastic decrease in percentage of CD19+-B-cells as well as CD20+-B-cells of total cells as well as per tissue area in all investigated lymphoid tissues when compared to the vehicle-treated animals without further significant decrease among the different treatment groups. There was significant decrease in activated germinal center B-cells in spleen and tonsils in all RG6035-treated animals compared to vehicle control.

Cell segmentation and subsequent thresholding for each marker allowed precise quantification of each B-cell subset. CD19+CD27-CD38−-Naïve B-cells (n=2), CD19+CD38+- or CD19+CD21−-activated germinal center B-cells (n=4), CD19+CD38-CD27+-memory B-cells (n=2), CD19+CD21+-naïve/memory B-cells (n=2), CD138+CD21−-plasma cells (n=2) in cervical lymph node, spleen, mandibular lymph node and tonsils. Two-Way ANOVA with multiple comparisons within each row (simple effects within rows, compared to vehicle control) was performed. Percentages of positive cell types were normalized to tissue area. *, p<0.05 compared to vehicle control. The results are shown in FIGS. 16 to 19.

One of ordinary skill in the art will understand that the therapeutically effective amount of RG6035 administered to a subject may depend upon a number of factors including pharmacodynamic characteristics, route of administration, frequency of treatment, and health, age, and weight of the subject to be treated and, with the information disclosed herein, will be able to determine the appropriate amount for each subject.

In certain embodiments of all aspects and embodiments of the current invention, the therapeutically effective amount is a dose chosen to improve efficacy and/or maintain efficacy and improve at least one of safety and tolerability. In some embodiments, the therapeutically effective amount is chosen to lower at least one side effect and simultaneously improve efficacy and/or maintain efficacy.

In certain embodiments, 1 mg/kg to 4 mg/kg, 1 mg/kg to 3.5 mg/kg, 1 mg/kg to 3.0 mg/kg, 1 mg/kg to 2.9 mg/kg, 1 mg/kg to 2.8 mg/kg, 1 mg/kg to 2.7 mg/kg, 1 mg/kg to 2.6 mg/kg, 1 mg/kg to 2.5 mg/kg, 1 mg/kg to 2.4 mg/kg, 1 mg/kg to 2.3 mg/kg, 1 mg/kg to 2.2 mg/kg, 1 mg/kg to 2.1 mg/kg, or 1 mg/kg to 2 mg/kg of RG6035 is administered to the subject relative to the body weight of the subject.

In certain embodiments, 1.25 mg/kg to 4 mg/kg, 1.25 mg/kg to 3.5 mg/kg, 1.25 mg/kg to 3 mg/kg, 1.25 mg/kg to 2.9 mg/kg, 1.25 mg/kg to 2.8 mg/kg, 1.25 mg/kg to 2.7 mg/kg, 1.25 mg/kg to 2.6 mg/kg, 1.25 mg/kg to 2.5 mg/kg, 1.25 mg/kg to 2.4 mg/kg, 1.25 mg/kg to 2.3 mg/kg, or 1.25 mg/kg to 2.2 mg/kg of RG6035 is administered to the subject relative to the body weight of the subject.

In certain embodiments, 1.5 mg/kg to 4 mg/kg, 1.5 mg/kg to 3.5 mg/kg, 1.5 mg/kg to 3 mg/kg, 1.5 mg/kg to 2.9 mg/kg, 1.5 mg/kg to 2.8 mg/kg, 1.5 mg/kg to 2.7 mg/kg, 1.5 mg/kg to 2.6 mg/kg, 1.5 mg/kg to 2.5 mg/kg, 1.5 mg/kg to 2.4 mg/kg, 1.5 mg/kg to 2.3 mg/kg, or 1.5 mg/kg to 2.2 mg/kg, of RG6035 is administered to the subject relative to the body weight of the subject.

In certain embodiments, 1.8 mg/kg to 4 mg/kg, 1.8 mg/kg to 3.5 mg/kg, 1.8 mg/kg to 3 mg/kg, 1.8 mg/kg to 2.9 mg/kg, 1.8 mg/kg to 2.8 mg/kg, 1.8 mg/kg to 2.7 mg/kg, 1.8 mg/kg to 2.6 mg/kg, 1.8 mg/kg to 2.5 mg/kg, 1.8 mg/kg to 2.4 mg/kg, 1.8 mg/kg to 2.3 mg/kg, or 1.8 mg/kg to 2.2 mg/kg, of RG6035 is administered to the subject relative to the body weight of the subject.

In certain embodiments, 1.9 mg/kg to 4 mg/kg, 1.9 mg/kg to 3.5 mg/kg, 1.9 mg/kg to 3 mg/kg, 1.9 mg/kg to 2.9 mg/kg, 1.9 mg/kg to 2.8 mg/kg, 1.9 mg/kg to 2.7 mg/kg, 1.9 mg/kg to 2.6 mg/kg, 1.9 mg/kg to 2.5 mg/kg, 1.9 mg/kg to 2.4 mg/kg, 1.9 mg/kg to 2.3 mg/kg, or 1.9 mg/kg to 2.2 mg/kg, of RG6035 is administered to the subject relative to the body weight of the subject.

In certain embodiments, 2 mg/kg to 4 mg/kg, 2 mg/kg to 3.5 mg/kg, 2 mg/kg to 3 mg/kg, 2 mg/kg to 2.9 mg/kg, 2 mg/kg to 2.8 mg/kg, 2 mg/kg to 2.7 mg/kg, 2 mg/kg to 2.6 mg/kg, 2 mg/kg to 2.5 mg/kg, 2 mg/kg to 2.4 mg/kg, 2 mg/kg to 2.3 mg/kg, or 2 mg/kg to 2.2 mg/kg, of RG6035 is administered to the subject relative to the body weight of the subject.

In certain embodiments, 1 mg/kg to 4 mg/kg, 1.1 mg/kg to 4 mg/kg, 1.2 mg/kg to 4 mg/kg, 1.3 mg/kg to 4 mg/kg, 1.4 mg/kg to 4 mg/kg, 1.5 mg/kg to 4 mg/kg, 1.6 mg/kg to 4 mg/kg, 1.7 mg/kg to 4 mg/kg, 1.8 mg/kg to 4 mg/kg, 1.9 mg/kg to 4 mg/kg, 2 mg/kg to 4 mg/kg, 2.1 mg/kg to 4 mg/kg, 2.2 mg/kg to 4 mg/kg, 2.3 mg/kg to 4 mg/kg, 2.4 mg/kg to 4 mg/kg, 2.5 mg/kg to 4 mg/kg, 2.6 mg/kg to 4 mg/kg, 2.7 mg/kg to 4 mg/kg, 2.8 mg/kg to 4 mg/kg, 2.9 mg/kg to 4 mg/kg, 3 mg/kg to 4 mg/kg, or 3.5 mg/kg to 4 mg/kg of RG6035 is administered to the subject relative to the body weight of the subject.

In certain embodiments, 1 mg/kg to 3 mg/kg, 1.1 mg/kg to 3 mg/kg, 1.2 mg/kg to 3 mg/kg, 1.3 mg/kg to 3 mg/kg, 1.4 mg/kg to 3 mg/kg, 1.5 mg/kg to 3 mg/kg, 1.6 mg/kg to 3 mg/kg, 1.7 mg/kg to 3 mg/kg, 1.8 mg/kg to 3 mg/kg, 1.9 mg/kg to 3 mg/kg, 2 mg/kg to 3 mg/kg, 2.1 mg/kg to 3 mg/kg, 2.2 mg/kg to 3 mg/kg, 2.3 mg/kg to 3 mg/kg, 2.4 mg/kg to 3 mg/kg, 2.5 mg/kg to 3 mg/kg, 2.6 mg/kg to 3 mg/kg, 2.7 mg/kg to 3 mg/kg, 2.8 mg/kg to 3 mg/kg, or 2.9 mg/kg to 3 mg/kg of RG6035 is administered to the subject relative to the body weight of the subject.

In certain embodiments, 1 mg/kg to 2.9 mg/kg, 1.1 mg/kg to 2.9 mg/kg, 1.2 mg/kg to 2.9 mg/kg, 1.3 mg/kg to 2.9 mg/kg, 1.4 mg/kg to 2.9 mg/kg, 1.5 mg/kg to 2.9 mg/kg, 1.6 mg/kg to 2.9 mg/kg, 1.7 mg/kg to 2.9 mg/kg, 1.8 mg/kg to 2.9 mg/kg, 1.9 mg/kg to 2.9 mg/kg, 2 mg/kg to 2.9 mg/kg, 2.1 mg/kg to 2.9 mg/kg, 2.2 mg/kg to 2.9 mg/kg, 2.3 mg/kg to 2.9 mg/kg, 2.4 mg/kg to 2.9 mg/kg, 2.5 mg/kg to 2.9 mg/kg, 2.6 mg/kg to 2.9 mg/kg, 2.7 mg/kg to 2.9 mg/kg, or 2.8 mg/kg to 2.9 mg/kg of RG6035 is administered to the subject relative to the body weight of the subject.

In certain embodiments, 1 mg/kg to 2.8 mg/kg, 1.1 mg/kg to 2.8 mg/kg, 1.2 mg/kg to 2.8 mg/kg, 1.3 mg/kg to 2.8 mg/kg, 1.4 mg/kg to 2.8 mg/kg, 1.5 mg/kg to 2.8 mg/kg, 1.6 mg/kg to 2.8 mg/kg, 1.7 mg/kg to 2.8 mg/kg, 1.8 mg/kg to 2.8 mg/kg, 1.9 mg/kg to 2.8 mg/kg, 2 mg/kg to 2.8 mg/kg, 2.1 mg/kg to 2.8 mg/kg, 2.2 mg/kg to 2.8 mg/kg, 2.3 mg/kg to 2.8 mg/kg, 2.4 mg/kg to 2.8 mg/kg, 2.5 mg/kg to 2.8 mg/kg, 2.6 mg/kg to 2.8 mg/kg, or 2.7 mg/kg to 2.8 mg/kg of RG6035 is administered to the subject relative to the body weight of the subject.

In certain embodiments, 1 mg/kg to 2.7 mg/kg, 1.1 mg/kg to 2.7 mg/kg, 1.2 mg/kg to 2.7 mg/kg, 1.3 mg/kg to 2.7 mg/kg, 1.4 mg/kg to 2.7 mg/kg, 1.5 mg/kg to 2.7 mg/kg, 1.6 mg/kg to 2.7 mg/kg, 1.7 mg/kg to 2.7 mg/kg, 1.8 mg/kg to 2.7 mg/kg, 1.9 mg/kg to 2.7 mg/kg, 2 mg/kg to 2.7 mg/kg, 2.1 mg/kg to 2.7 mg/kg, 2.2 mg/kg to 2.7 mg/kg, 2.3 mg/kg to 2.7 mg/kg, 2.4 mg/kg to 2.7 mg/kg, 2.5 mg/kg to 2.8 mg/kg, or 2.6 mg/kg to 2.7 mg/kg of RG6035 is administered to the subject relative to the body weight of the subject.

In certain embodiments, 1 mg/kg to 4 mg/kg, 1.1 mg/kg to 4 mg/kg, 1.2 mg/kg to 4 mg/kg, 1.3 mg/kg to 4 mg/kg, 1.4 mg/kg to 4 mg/kg, 1.5 mg/kg to 4 mg/kg, 1.5 mg/kg to 3.5 mg/kg, 1.5 mg/kg to 3 mg/kg, 1.5 mg/kg to 2.9 mg/kg, 1.5 mg/kg to 2.8 mg/kg, 1.5 mg/kg to 2.7 mg/kg, 1.6 mg/kg to 4 mg/kg, 1.6 mg/kg to 3.5 mg/kg, 1.6 mg/kg to 3 mg/kg, 1.6 mg/kg to 2.9 mg/kg, 1.6 mg/kg to 2.8 mg/kg, 1.6 mg/kg to 2.7 mg/kg, 1.7 mg/kg to 4 mg/kg, 1.7 mg/kg to 3.5 mg/kg, 1.7 mg/kg to 3 mg/kg, 1.7 mg/kg to 2.9 mg/kg, 1.7 mg/kg to 2.8 mg/kg, 1.7 mg/kg to 2.7 mg/kg, 1.8 mg/kg to 4 mg/kg, 1.8 mg/kg to 3.5 mg/kg, 1.8 mg/kg to 3 mg/kg, 1.8 mg/kg to 2.9 mg/kg, 1.8 mg/kg to 2.8 mg/kg, 1.8 mg/kg to 2.7 mg/kg, 1.9 mg/kg to 4 mg/kg, 1.9 mg/kg to 3.5 mg/kg, 1.9 mg/kg to 3 mg/kg, 1.9 mg/kg to 2.9 mg/kg, 1.9 mg/kg to 2.8 mg/kg, or 1.9 mg/kg to 2.7 mg/kg of RG6035 is administered to the subject relative to the body weight of the subject.

In some embodiments, 1 mg/kg of RG6035 is administered to the subject relative to body weight of the subject. In some embodiments, 1.1 mg/kg of RG6035 is administered to the subject relative to body weight of the subject. In some embodiments, 1.2 mg/kg of RG6035 is administered to the subject relative to body weight of the subject. In some embodiments, 1.3 mg/kg of RG6035 is administered to the subject relative to body weight of the subject. In some embodiments, 1.4 mg/kg of RG6035 is administered to the subject relative to body weight of the subject. In some embodiments, 1.5 mg/kg of RG6035 is administered to the subject relative to body weight of the subject. In some embodiments, 1.6 mg/kg of RG6035 is administered to the subject relative to body weight of the subject. In some embodiments, 1.7 mg/kg of RG6035 is administered to the subject relative to body weight of the subject. In some embodiments, 1.8 mg/kg of RG6035 is administered to the subject relative to body weight of the subject. In some embodiments, 1.9 mg/kg of RG6035 is administered to the subject relative to body weight of the subject. In some embodiments, 2 mg/kg of RG6035 is administered to the subject relative to body weight of the subject. In some embodiments, 2.1 mg/kg of RG6035 is administered to the subject relative to body weight of the subject. In some embodiments, 2.2 mg/kg of at RG6035 is administered to the subject relative to body weight of the subject. In some embodiments, 2.3 mg/kg of RG6035 is administered to the subject relative to body weight of the subject. In some embodiments, 2.4 mg/kg of RG6035 is administered to the subject relative to body weight of the subject. In some embodiments, 2.5 mg/kg of RG6035 is administered to the subject relative to body weight of the subject. In some embodiments, 2.6 mg/kg of RG6035 is administered to the subject relative to body weight of the subject. In some embodiments, 2.7 mg/kg of RG6035 is administered to the subject relative to body weight of the subject. In some embodiments, 2.8 mg/kg of RG6035 is administered to the subject relative to body weight of the subject. In some embodiments, 2.9 mg/kg of RG6035 is administered to the subject relative to body weight of the subject. In some embodiments, 3 mg/kg of RG6035 is administered to the subject relative to body weight of the subject.

In certain embodiments, a dose of 70 mg to 300 mg, 70 mg to 280 mg, 70 mg to 260 mg, 70 mg to 250 mg, 70 mg to 240 mg, 70 mg to 230 mg, 70 mg to 220 mg, 70 mg to 210 mg, 70 mg to 200 mg, 70 mg to 190 mg, 70 mg to 180 mg, 70 mg to 170 mg, 70 mg to 160 mg, or 70 mg to 150 mg of RG6035 is administered to the subject.

In certain embodiments, a dose of 75 mg to 300 mg, 75 mg to 280 mg, 75 mg to 260 mg, 75 mg to 250 mg, 75 mg to 240 mg, 75 mg to 230 mg, 75 mg to 220 mg, 75 mg to 210 mg, 75 mg to 200 mg, 75 mg to 190 mg, 75 mg to 180 mg, 75 mg to 170 mg, 75 mg to 160 mg, or 75 mg to 150 mg of RG6035 is administered to the subject.

In certain embodiments, a dose of 100 mg to 300 mg, 100 mg to 280 mg, 100 mg to 260 mg, 100 mg to 250 mg, 100 mg to 240 mg, 100 mg to 230 mg, 100 mg to 220 mg, 100 mg to 210 mg, 100 mg to 200 mg, 100 mg to 190 mg, 100 mg to 180 mg, 100 mg to 170 mg, 100 mg to 160 mg, or 100 mg to 150 mg of RG6035 is administered to the subject.

In certain embodiments, a dose of 110 mg to 300 mg, 110 mg to 280 mg, 110 mg to 260 mg, 110 mg to 250 mg, 110 mg to 240 mg, 110 mg to 230 mg, 110 mg to 220 mg, 110 mg to 210 mg, 110 mg to 200 mg, 110 mg to 190 mg, 110 mg to 180 mg, 110 mg to 170 mg, 110 mg to 160 mg, or 110 mg to 150 mg of RG6035 is administered to the subject.

In certain embodiments, a dose of 120 mg to 300 mg, 120 mg to 280 mg, 120 mg to 260 mg, 120 mg to 250 mg, 120 mg to 240 mg, 120 mg to 230 mg, 120 mg to 220 mg, 120 mg to 210 mg, 120 mg to 200 mg, 120 mg to 190 mg, 120 mg to 180 mg, 120 mg to 170 mg, 120 mg to 160 mg, or 120 mg to 150 mg of RG6035 is administered to the subject.

In certain embodiments, a dose of 130 mg to 300 mg, 130 mg to 280 mg, 130 mg to 260 mg, 130 mg to 250 mg, 130 mg to 240 mg, 130 mg to 230 mg, 130 mg to 220 mg, 130 mg to 210 mg, 130 mg to 200 mg, 130 mg to 190 mg, 130 mg to 180 mg, 130 mg to 170 mg, 130 mg to 160 mg, or 130 mg to 150 mg of RG6035 is administered to the subject.

In certain embodiments, a dose of 140 mg to 300 mg, 140 mg to 280 mg, 140 mg to 260 mg, 140 mg to 250 mg, 140 mg to 240 mg, 140 mg to 230 mg, 140 mg to 220 mg, 140 mg to 210 mg, 140 mg to 200 mg, 140 mg to 190 mg, 140 mg to 180 mg, 140 mg to 170 mg, 140 mg to 160 mg, or 140 mg to 150 mg of RG6035 is administered to the subject.

In certain embodiments, a dose of 70 mg to 300 mg, 75 mg to 300 mg, 100 mg to 300 mg, 110 mg to 300 mg, 120 mg to 300 mg, 130 mg to 300 mg, 140 mg to 300 mg, 150 mg to 300 mg, 160 mg to 300 mg, 170 mg to 300 mg, 180 mg to 300 mg, 190 mg to 300 mg, 200 mg to 300 mg, 210 mg to 300 mg, 220 mg to 300 mg, 230 mg to 300 mg, 240 mg to 300 mg, 250 mg to 300 mg, 260 mg to 300 mg, 270 mg to 300 mg, 280 mg to 300 mg, or 290 mg to 300 mg of RG6035 is administered to the subject.

In certain embodiments, a dose of 70 mg to 290 mg, 75 mg to 290 mg, 100 mg to 290 mg, 110 mg to 290 mg, 120 mg to 290 mg, 130 mg to 290 mg, 140 mg to 290 mg, 150 mg to 290 mg, 160 mg to 290 mg, 170 mg to 290 mg, 180 mg to 290 mg, 190 mg to 290 mg, 200 mg to 290 mg, 210 mg to 290 mg, 220 mg to 290 mg, 230 mg to 290 mg, 240 mg to 290 mg, 250 mg to 290 mg, 260 mg to 290 mg, 270 mg to 290 mg, or 280 mg to 290 mg of RG6035 is administered to the subject.

In certain embodiments, a dose of 70 mg to 280 mg, 75 mg to 300 mg, 100 mg to 280 mg, 110 mg to 280 mg, 120 mg to 280 mg, 130 mg to 280 mg, 140 mg to 280 mg, 150 mg to 280 mg, 160 mg to 280 mg, 170 mg to 280 mg, 180 mg to 280 mg, 190 mg to 280 mg, 200 mg to 280 mg, 210 mg to 280 mg, 220 mg to 280 mg, 230 mg to 280 mg, 240 mg to 280 mg, 250 mg to 280 mg, 260 mg to 280 mg, or 270 mg to 280 mg of RG6035 is administered to the subject.

In certain embodiments, a dose of 70 mg to 270 mg, 75 mg to 270 mg, 100 mg to 270 mg, 110 mg to 270 mg, 120 mg to 270 mg, 130 mg to 270 mg, 140 mg to 270 mg, 150 mg to 270 mg, 160 mg to 270 mg, 170 mg to 270 mg, 180 mg to 270 mg, 190 mg to 270 mg, 200 mg to 270 mg, 210 mg to 270 mg, 220 mg to 270 mg, 230 mg to 270 mg, 240 mg to 270 mg, 250 mg to 270 mg, or 260 mg to 270 mg of RG6035 is administered to the subject.

In certain embodiments, a dose of 70 mg to 260 mg, 75 mg to 260 mg, 100 mg to 260 mg, 110 mg to 260 mg, 120 mg to 260 mg, 130 mg to 260 mg, 140 mg to 260 mg, 150 mg to 260 mg, 160 mg to 260 mg, 170 mg to 260 mg, 180 mg to 260 mg, 190 mg to 260 mg, 200 mg to 260 mg, 210 mg to 260 mg, 220 mg to 260 mg, 230 mg to 260 mg, 240 mg to 260 mg, or 250 mg to 260 mg of RG6035 is administered to the subject.

In certain embodiments, a dose of 70 mg to 250 mg, 75 mg to 250 mg, 100 mg to 250 mg, 110 mg to 250 mg, 120 mg to 250 mg, 130 mg to 250 mg, 140 mg to 250 mg, 150 mg to 250 mg, 160 mg to 250 mg, 170 mg to 250 mg, 180 mg to 250 mg, 190 mg to 250 mg, 200 mg to 250 mg, 210 mg to 250 mg, 220 mg to 250 mg, 230 mg to 250 mg, or 240 mg to 250 mg of RG6035 is administered to the subject.

In certain embodiments, a dose of 70 mg to 240 mg, 75 mg to 240 mg, 100 mg to 240 mg, 110 mg to 240 mg, 120 mg to 240 mg, 130 mg to 240 mg, 140 mg to 240 mg, 150 mg to 240 mg, 160 mg to 240 mg, 170 mg to 240 mg, 180 mg to 240 mg, 190 mg to 240 mg, 200 mg to 240 mg, 210 mg to 240 mg, 220 mg to 240 mg, or 230 mg to 240 mg of RG6035 is administered to the subject.

In certain embodiments, a dose of 70 mg to 230 mg, 75 mg to 230 mg, 100 mg to 230 mg, 110 mg to 230 mg, 120 mg to 230 mg, 130 mg to 230 mg, 140 mg to 230 mg, 150 mg to 230 mg, 160 mg to 230 mg, 170 mg to 230 mg, 180 mg to 230 mg, 190 mg to 230 mg, 200 mg to 230 mg, 210 mg to 230 mg, or 220 mg to 230 mg of RG6035 is administered to the subject.

In certain embodiments, a dose of 70 mg to 220 mg, 75 mg to 220 mg, 100 mg to 220 mg, 110 mg to 220 mg, 120 mg to 220 mg, 130 mg to 220 mg, 140 mg to 220 mg, 150 mg to 220 mg, 160 mg to 220 mg, 170 mg to 220 mg, 180 mg to 220 mg, 190 mg to 220 mg, 200 mg to 220 mg, or 210 mg to 220 mg of RG6035 is administered to the subject.

In certain embodiments, a dose of 70 mg to 210 mg, 75 mg to 210 mg, 100 mg to 210 mg, 110 mg to 210 mg, 120 mg to 210 mg, 130 mg to 210 mg, 140 mg to 210 mg, 150 mg to 210 mg, 160 mg to 210 mg, 170 mg to 210 mg, 180 mg to 2100 mg, 190 mg to 210 mg, or 200 mg to 210 mg of RG6035 is administered to the subject.

In certain embodiments, a dose of 70 mg to 200 mg, 75 mg to 200 mg, 100 mg to 200 mg, 110 mg to 200 mg, 120 mg to 200 mg, 130 mg to 200 mg, 140 mg to 200 mg, 150 mg to 200 mg, 160 mg to 200 mg, 170 mg to 200 mg, 180 mg to 200 mg, or 190 mg to 200 mg of RG6035 is administered to the subject.

In certain embodiments, a dose of 70 mg to 190 mg, 75 mg to 190 mg, 100 mg to 190 mg, 110 mg to 190 mg, 120 mg to 190 mg, 130 mg to 190 mg, 140 mg to 190 mg, 150 mg to 190 mg, 160 mg to 190 mg, 170 mg to 190 mg, or 180 mg to 190 mg of RG6035 is administered to the subject.

In certain embodiments, a dose of 70 mg to 180 mg, 75 mg to 180 mg, 100 mg to 180 mg, 110 mg to 180 mg, 120 mg to 180 mg, 130 mg to 180 mg, 140 mg to 180 mg, 150 mg to 180 mg, 160 mg to 180 mg, or 170 mg to 180 mg of RG6035 is administered to the subject.

In certain embodiments, a dose of 70 mg to 170 mg, 75 mg to 170 mg, 100 mg to 170 mg, 110 mg to 170 mg, 120 mg to 170 mg, 130 mg to 170 mg, 140 mg to 170 mg, 150 mg to 170 mg, or 160 mg to 170 mg of RG6035 is administered to the subject.

In certain embodiments, a dose of 70 mg to 160 mg, 75 mg to 160 mg, 100 mg to 160 mg, 110 mg to 160 mg, 120 mg to 160 mg, 130 mg to 160 mg, 140 mg to 160 mg, or 150 mg to 160 mg of RG6035 is administered to the subject.

In certain embodiments, a dose of 70 mg to 150 mg, 75 mg to 150 mg, 100 mg to 150 mg, 110 mg to 150 mg, 120 mg to 150 mg, 130 mg to 150 mg, or 140 mg to 150 mg of RG6035 is administered to the subject.

In certain embodiments, a dose of 70 mg to 300 mg, 75 mg to 300 mg, 100 mg to 300 mg, 110 mg to 300 mg, 120 mg to 300 mg, 130 mg to 300 mg, 140 mg to 300 mg, 150 mg to 300 mg, 70 mg to 280 mg, 75 mg to 280 mg, 100 mg to 280 mg, 110 mg to 280 mg, 120 mg to 280 mg, 130 mg to 280 mg, 140 mg to 280 mg, 150 mg to 280 mg, 70 mg to 270 mg, 75 mg to 270 mg, 100 mg to 270 mg, 110 mg to 270 mg, 120 mg to 270 mg, 130 mg to 270 mg, 140 mg to 270 mg, 150 mg to 270 mg, 70 mg to 260 mg, 75 mg to 260 mg, 100 mg to 260 mg, 110 mg to 260 mg, 120 mg to 260 mg, 130 mg to 260 mg, 140 mg to 260 mg, 150 mg to 260 mg, 70 mg to 250 mg, 75 mg to 250 mg, 100 mg to 250 mg, 110 mg to 250 mg, 120 mg to 250 mg, 130 mg to 250 mg, 140 mg to 250 mg, 150 mg to 250 mg, 70 mg to 240 mg, 75 mg to 240 mg, 100 mg to 240 mg, 110 mg to 240 mg, 120 mg to 240 mg, 130 mg to 240 mg, 140 mg to 240 mg, 150 mg to 240 mg, 70 mg to 230 mg, 75 mg to 230 mg, 100 mg to 230 mg, 110 mg to 230 mg, 120 mg to 230 mg, 130 mg to 230 mg, 140 mg to 230 mg, 150 mg to 230 mg, 70 mg to 220 mg, 75 mg to 220 mg, 100 mg to 220 mg, 110 mg to 220 mg, 120 mg to 220 mg, 130 mg to 220 mg, 140 mg to 220 mg, 150 mg to 220 mg, 70 mg to 210 mg, 75 mg to 210 mg, 100 mg to 210 mg, 110 mg to 210 mg, 120 mg to 210 mg, 130 mg to 210 mg, 140 mg to 210 mg, 150 mg to 210 mg, 70 mg to 200 mg, 75 mg to 200 mg, 100 mg to 200 mg, 110 mg to 200 mg, 120 mg to 200 mg, 130 mg to 200 mg, 140 mg to 200 mg or 150 mg to 200 mg of RG6035 is administered to the subject.

In some embodiments, a dose of 70 mg of RG6035 is administered to the subject. In some embodiments, a dose of 75 mg of RG6035 is administered to the subject. In some embodiments, a dose of 100 mg of RG6035 is administered to the subject. In some embodiments, a dose of 110 mg of RG6035 is administered to the subject. In some embodiments, a dose of 120 mg of RG6035 is administered to the subject. In some embodiments, a dose of 130 mg of RG6035 is administered to the subject. In some embodiments, a dose of 140 mg of RG6035 is administered to the subject. In some embodiments, a dose of 150 mg of RG6035 is administered to the subject. In some embodiments, a dose of 160 mg of RG6035 is administered to the subject. In some embodiments, a dose of 170 mg of RG6035 is administered to the subject. In some embodiments, a dose of 180 mg of RG6035 is administered to the subject. In some embodiments, a dose of 190 mg of RG6035 is administered to the subject. In some embodiments, a dose of 200 mg of at RG6035 is administered to the subject. In some embodiments, a dose of 210 mg of RG6035 is administered to the subject. In some embodiments, a dose of 220 mg of RG6035 is administered to the subject. In some embodiments, a dose of 230 mg of RG6035 is administered to the subject. In some embodiments, a dose of 240 mg of RG6035 is administered to the subject. In some embodiments, a dose of 250 mg of RG6035 is administered to the subject. In some embodiments, a dose of 260 mg of RG6035 is administered to the subject. In some embodiments, a dose of 280 mg of RG6035 is administered to the subject relative. In some embodiments, a dose of 300 mg of RG6035 is administered to the subject.

### Therapeutic Application

The ability to achieve sufficient brain penetration while maintaining a safe therapeutic window is key for the development of brain-targeted antibodies. To this end, RG6035, an Fc-region-effector-function-silent bispecific modular fusion protein of a huTfR1-directed brain shuttle and obinutuzumab, was designed. In a head-to-head comparison in human and mouse in vitro models, and in in vivo mouse and cynomolgus monkey preclinical models, RG6035 demonstrated a superior safety profile compared to an Fc-region-effector-function-competent construct (BS-obinutuzumab) while having the ability to achieve higher brain exposures compared to a ‘normal’, non-TfR1 binding antibody. The Fc-region-effector-function-silencing PGLALA mutation prevents binding of RG6035 to Fc gamma receptors and therefore the activation of immune effector cells and at the same time does not affect TfR1 and CD20 target binding. Uptake and transcytosis of RG6035 in TfR1-expressing cells is also maintained when compared to Fc-region-effector-function-competent constructs. Furthermore, RG6035 has potent direct B-cell death induction capacity, demonstrated by fast and potent reduction of human B-cell lymphoma cells as well as primary human B-cells in WBA, tonsil derived cell cultures and human CSF cultured PBMCs. It has to be pointed out that in a B-cell lymphoma assay that assessed direct B-cell killing without immune effector functions, comparable efficacy was demonstrated between RG6035 and BS-obinutuzumab. In an ex vivo CSF assay, RG6035, like BS-obinutuzumab, was effective in depleting B-cells. Thus, the data presented herein demonstrates that the Fc-region-effector-function-silencing mutations and the BS module do not hinder CD20 target binding as well as the direct B-cell killing activity, and the anti-CD20 targeting does not interfere with active brain uptake in RG6035.

TfR1 is widely expressed in multiple tissues, which could lead to systemic target-mediated drug disposition (TMDD) as well as safety implications due to binding of the TfR1 receptor in undesired locations in the body.[30a, 31a] It has now been found that potential safety concerns related to TfR1 targeting in the periphery are mitigated in RG6035 [see also 25a].

IRRs are an inherent risk of an antibody-based therapy, representing a major obstacle for mAb clinical development. IRR assessment and steps to reduce IRRs are, therefore, essential for mAb clinical development. In RG6035 IRRs and effects on RBC precursors have been advantageously affected, i.e. addressed, by the specific design of Fc-region-effector-function-function-silenced RG6035 as shown in humanized CD20 mice. Hypoactivity, temperature decrease, and cytokine release, all indicative of IRRs, as well as reduced reticulocyte counts occurred only in mice treated with Fc-region-effector-function-competent BS-obinutuzumab [see also 27a] It has to be pointed out that steric hindrance may attenuate Fc-gamma-receptor interactions but only in the case of a brain-restricted therapeutic target, but might not hold true when both targets (e.g., CD20 and TfR1) are expressed peripherally. Interestingly, obinutuzumab without the BS molecule does not cause IRRs in the transgenic mouse models, despite the fact that this molecule retains the effector function and typically shows IRRs in humans.

In a human WBA, proinflammatory cytokine release was demonstrated with Fc-region-effector-function-competent BS-obinutuzumab, which was not present in the Fc-region-effector-function-silenced RG6035. Importantly, due to the type II killing of B-cells, Fc-region-effector-function-silenced RG6035 still has B-cell-killing properties. Without being bound by this theory, it is assumed that direct type II B-cell killing plays a major role in the CNS as in the brain lower numbers of effector cells are present. Additionally, from a safety point of view, Fc-region-effector-function-mediated effector activation and subsequent B-cell killing in the CNS by the same mechanism would induce unfavorable cytokine release.

RG6035 has improved BBB penetration properties and gains access to the CNS compared to a non-shuttled anti-CD20 antibody. Vascular uptake and brain penetration was demonstrated in vivo via enzyme-linked immunosorbent assay (ELISA) and immunohistochemistry in the mouse PK study. Without being bound by this theory, it is assumed that the improved brain penetration combined with the properties for potent Fc-region-effector-function-independent extravascular B-cell depletion by direct B-cell killing translates to clinical efficacy for MS patients, by preventing or slowing clinical progression.

In the cynomolgus monkey and transgenic mouse models, secondary lymphoid tissues containing B-cell populations were used as a surrogate to model difficult to treat B-cells, such as in MS. In the huCD20 transgenic mouse single-dose PK/PD study, a strong B-cell depletion in blood, spleen, or lymph nodes was not observed, but B-cell depletion was demonstrated in these organs in multiple dose studies on naïve mice. Without being bound by this theory, it is assumed that in contrast to the non-human primate, huCD20 transgenic mice require multiple administrations of anti-CD20 antibodies to achieve strong B-cell depletion. Possible reasons for this may be the lower density of human CD20 on murine B-cells in the huCD20 transgenic mouse, or the superior affinity of the surrogate anti-murine TfR1 brain shuttle module in mice relative to the anti-human TfR1 brain shuttle module in cynomolgus monkeys, leading to increased TMDD via TfR1. In the huCD20 mouse, it was demonstrated that Fc-region-effector-function-silent RG6035 was effective in depleting GC class-switched B cells, which are described as an important component of MS pathophysiology. [33a]

The potency of RG6035 to deplete B-cells was assessed in vivo in the cynomolgus monkey. In separate single-dose PK studies, RG6035 was well tolerated and demonstrated an improved safety profile in comparison to Fc-region-effector-function-competent BS-obinutuzumab, based on clinical signs, cytokine levels, and clinical pathology. This supported the in vitro and in vivo mouse data, which indicated an increased risk for adverse effects with an Fc-region-effector-function-competent BS construct. With RG6035, there was rapid and strong B-cell count reduction in cynomolgus monkey blood following administration. While all animals developed ADAs independent of the Fc-variant, it is known that immunogenicity in the cynomolgus monkey is poorly predictive for that in humans.[34a]

Without being bound by this theory it is assumed that RG6035 would gain access to brain-compartmentalized neuroinflammation and, through potent Fc-region-effector-function-independent extravascular B-cell depletion, would lead to better clinical efficacy for patients with MS, either preventing or slowing clinical progression.

It is herein assumed that a subject in need of treatment with RG6035 will have a body weight in the range of 70 kg to 75 kg to allow a change of the denoted doses from mg to mg/kg and vice versa.

### Thus, the Current Invention Encompasses at Least the Following Independent and Dependent Embodiments

1. RG6035 for use in treating multiple sclerosis.
1a. RG6035 for use as a medicament in the treatment of multiple sclerosis.
1b. RG6035 for use in the treatment of multiple sclerosis.
2. Use of RG6035 in the manufacture of a medicament for treating multiple sclerosis.
2a. Use of RG6035 in the manufacture of a medicament for the treatment of multiple sclerosis.
3. RG6035 for use in depleting brain sequestered B-cells expressing CD20.
4. A method of treating an individual having multiple sclerosis comprising administering to the individual an effective amount of RG6035.
5. A method for depleting CD20 expressing circulating B-cells or CD20 expressing brain sequestered B-cells or CD20 expressing B-cells in the CSF or all of the before in an individual comprising administering to the individual an effective amount of RG6035 to deplete CD20 expressing circulating B-cells or CD20 expressing brain sequestered B-cells or CD20 expressing B-cells in the CSF or all of these B-cells.
6. The method according to embodiment 5, wherein the individual has multiple sclerosis.
7. A method of treating multiple sclerosis in a human comprising administering to the human a therapeutically effective amount of RG6035, which binds to human CD20 and depletes B-cells, and wherein the antibody is not conjugated to a cytotoxic agent.
8. The method or use according to any one of embodiments 1 to 7, wherein the multiple sclerosis is relapsing multiple sclerosis or progressive multiple sclerosis.
9. The method or use according to any one of embodiments 1 to 8, wherein the multiple sclerosis is primary progressive multiple sclerosis or secondary progressive multiple sclerosis.
10. The method or use according to any one of embodiments 1 to 9, wherein the multiple sclerosis is secondary progressive multiple sclerosis.
11. The method or use according to any one of embodiments 1 to 10, wherein RG6035 is administered at an effective dose to deplete more than 60% or more than 80% or more than 95% of B-cells.
12. The method or use according to any one of embodiments 1 to 10, wherein RG6035 is administered at an effective dose to reach ≥60% or ≥80% or ≥95% of B-cell depletion.
12a. The method or use according to any one of embodiments 1 to 10, wherein RG6035 is administered chronically (at least 1 year) at an effective dose to reach ≥95% of B-cell depletion.
13. The method or use according to any one of embodiments 11 to 12a, wherein the depletion is in the brain or/and the CSF.
14. The method or use according to any one of embodiments 11 to 13, wherein the depletion is in the CSF.
15. The method or use according to any one of embodiments 11 to 14, wherein the depletion is achieved within 8 weeks after start of the administration.
16. The method or use according to any one of embodiments 11 to 15, wherein the depletion is achieved within 6 weeks after start of the administration.
17. The method or use according to any one of embodiments 11 to 16, wherein the depletion is achieved within 5 weeks after start of the administration.
18. The method or use according to any one of embodiments 11 to 17, wherein the depletion is achieved within 4 weeks after start of the administration.
19. The method or use according to any one of embodiments 1 to 18, wherein RG6035 is administered at a non-toxic dose to achieve a reduction in CNS compartmentalized inflammation.
20. The method or use according to any one of embodiments 1 to 19, wherein RG6035 is administered at a non-toxic dose to achieve a reduction of B-cell influx from the blood into the brain and a direct local B-cell killing in the brain.
21. The method or use according to any one of embodiments 1 to 20, wherein RG6035 is administered at a non-toxic dose to achieve a reduction of disability progression.
22. The method or use according to any one of embodiments 1 to 21, wherein RG6035 is administered at a non-toxic dose to achieve 0.1% or more, 0.5% or more, 0.75% or more, 0.85% or more, 0.9% or more, or 1% or more of serum concentration of RG6035 in the cortex.
23. The method or use according to any one of embodiments 1 to 22, wherein RG6035 is administered at a non-toxic dose to achieve 0.1% or more, 0.5% or more, 0.75% or more, 0.85% or more, 0.9% or more, 1% or more, 1.25% or more, or 1.5% or more of serum concentration of RG6035 in the CSF.
24. The method or use according to any one of embodiments 1 to 22, wherein RG6035 is administered at a non-toxic dose to achieve 0.1% or more, 0.5% or more, 0.75% or more, 0.85% or more, 0.9% or more, 1% or more, 1.25% or more, or 1.5% or more of serum concentration of RG6035 across different brain regions (excluding meninges) and in the CSF.
25. The method or use according to any one of embodiments 1 to 24, wherein RG6035 is administered at a non-toxic dose to achieve a concentration of RG6035 of 0.006 μg/mL or more, 0.025 μg/mL or more, 0.035 μg/mL or more, 0.045 μg/mL or more, 0.055 μg/mL or more, or 0.07 μg/mL or more in the CSF.
26. The method or use according to any one of embodiments 1 to 25, wherein RG6035 is administered at a non-toxic dose to achieve a depletion of the resident B-cell population in the brain.
27. The method or use according to embodiment 26, wherein the depletion is achieved by induction of direct B-cell death of immunologically active B-cells in an Fc-region-effector-function-independent manner.
28. The method or use according to any one of embodiments 1 to 27, wherein the B-cells are CD19 positive B-cells.
29. The method or use according to any one of embodiments 1 to 28, wherein RG6035 is administered at a second dose after more than 60% or more than 80% or more than 95% of B-cells have been depleted from the CSF to maintain the B-cell depletion in the CSF at said level.
29a. The method or use according to any one of embodiments 1 to 28, wherein RG6035 is administered chronically (at least 1 year) at a second dose after more than 95% of B-cells have been depleted from the CSF to maintain the B-cell depletion in the CSF at said level.
30. The method or use according to any one of embodiments 1 to 29a, wherein the B-cell depletion is determined compared to the B-cell number or level or concentration prior to the first administration of RG6035.
31. The method or use according to any one of embodiments 1 to 30, wherein RG6035 is administered as a monotherapy.
32. The method or use according to any one of embodiments 1 to 30, wherein RG6035 is administered in combination with a second therapeutic agent that depletes B-cells.
33. The method or use according to embodiment 32, wherein the second therapeutic agent is administered once prior to the administration of RG6035.
34. The method or use according to embodiment 32, wherein the second therapeutic agent is administered at the same time as RG6035.
35. The method or use according to any one of embodiments 1 to 34, wherein RG6035 is administered in an effective amount in the range of 75-300 mg, or 100-275 mg, or 125-250 mg, or 140-210 mg, or 150-200 mg per administration.
36. The method or use according to any one of embodiments 1 to 35, wherein RG6035 is administered in an effective amount of about 150 mg or about 200 mg per administration.
37. The method or use according to any one of embodiments 1 to 34, wherein RG6035 is administered in an effective amount in the range of 1-4 mg/kg, or 1.25-3.75 mg/kg, or 1.5-3.25 mg/kg, or 1.75-3 mg/kg or 2-2.85 mg/kg relative to the weight of the subject per administration.
38. The method or use according to any one of embodiments 1 to 34 and 37, wherein RG6035 is administered in an effective amount of about 2 mg/kg or about 2.85 mg/kg relative to the weight of the subject per administration.
39. The method or use according to any one of embodiments 1 to 38, wherein the effective amount is administered as a dose.
40. The method or use according to any one of embodiments 1 to 39, wherein the administration is once every week, or once every second week, or one every four weeks.
41. The method or use according to any one of embodiments 1 to 40, wherein the administration is once every four weeks.
42. The method or use according to any one of embodiments 1 to 41, wherein the administration is started as once every week and changed to once every four weeks after more than 95% of B-cells have been depleted from the CSF to maintain the B-cell depletion in the CSF at said level.
42a. The method or use according to any one of embodiments 1 to 42, wherein the administration is started as once every two weeks and changed to once every four weeks after more than 95% of B-cells have been depleted from the CSF to maintain the B-cell depletion in the CSF at said level.
43. The method or use according to any one of embodiments 41 to 42a, wherein the administration for once a week or for once every two weeks is for up to 8 administrations.
44. The method or use according to any one of embodiments 41 to 43, wherein the administration for once a week or for once every two wells is for up to 6 administrations.
45. The method or use according to any one of embodiment 41 to 44, wherein the administration for once a week or for once every two weeks is for up to 5 administrations.
46. The method or use according to any one of embodiments 41 to 45, wherein the administration for once a week or for once every two weeks is for up to 4 administrations.
47. The method or use according to any one of embodiments 29 to 46, wherein the second dose is in the range of 20-300 mg, or 50-275 mg, or 75-250 mg, or 100-210 mg, or 150-200 mg, or about 150 mg per administration.
48. The method or use according to any one of embodiments 29 to 46, wherein the second dose is in the range of 0.25-4 mg/kg, or 0.5-3.75 mg/kg, or 0.75-3.25 mg/kg, or 1.00-3 mg/kg, or 1.25-2.75 mg/kg, or 1.50-2.50 mg/kg, or about 2 mg/kg relative to the weight of the subject per administration.
49. The method or use according to any one of embodiments 1 to 48, wherein the administration of RG6035 is intravenously or subcutaneously.
50. The method or use according to any one of embodiments 1 to 48, wherein the administration of RG6035 is subcutaneously.

### Pharmacokinetic and Pharmacodynamic Experimental Results

Herein is shown an improved BBB penetration and B-cell killing with RG6035. This has been shown using a pharmacokinetic (PK) and pharmacodynamic (PD) study in cynomolgus monkeys. To our knowledge, there are no reliable disease models known in the art for B-cell depletion in the brain, let alone for the use of a brain shuttle antibody.

### Pharmacokinetics in Serum

In a first experiment, RG6035 was administered to cynomolgus monkeys via intravenous administration to establish its pharmacokinetic/pharmacodynamic relationship in both cerebrospinal fluid and blood. It has been found that RG6035 was well tolerated and showed efficacy in reaching the brain, and depleting B-cells in blood and secondary lymphoid tissues.

After a single IV administration of RG6035 to cynomolgus monkeys at 0.3 mg/kg, 1 mg/kg or 10 mg/kg in three Groups (3, 4 and 6, respectively), PK in serum was quantified up to 288 hours post-dose. RG6035 showed non-linear PK behavior attributed to target-mediated drug disposition (TMDD), with accelerated drug clearance at 0.3 mg/kg versus 10 mg/kg. NCA PK parameters in serum were calculated for each animal as indicated in Table 6.

TABLE 6
Individual mean PK parameters of RG6035 in serum after
IV administration of RG6035 at 0.3 mg/kg, 1 mg/kg and 10 mg/kg
in cynomolgus monkeys; AUC_{0-inf}, area under the concentration-
time curve from time 0 to infinity; C₀, initial or back-extrapolated
concentration following rapid IV injection; CL, clearance; Cₘₐₓ,
maximum concentration observed; IV, intravenous; PK, pharmacokinetic;
Vₛₛ, volume of distribution under steady-state conditions.

| Group | 3 | | 4 | | 6 | |
|---|---|---|---|---|---|---|
| Dose [mg/ | 0.3 | | 1 | | 10 | |
| kg] | | | | | | |
| Subject | 3501 | 3502 | 4501 | 4502 | 5501 | 5502 |
| C(0) [μg/ | 5.91 | 3.61 | 10.7 | 23.9 | 533 | 241 |
| mL] | | | | | | |
| Cₘₐₓ [μg/ | 4.61 | 3.42 | 15.0 | 21.7 | 490 | 222 |
| mL] | | | | | | |
| Cₘₐₓ/Dose | 15.4 | 11.4 | 15.0 | 21.7 | 49.0 | 22.2 |
| [[μg/mL]/ | | | | | | |
| [mg/kg]] | | | | | | |
| AUC_{0-inf} | 47.4 | 59.8 | 533 | 498 | 13000 | 5660 |
| [hr*μg/mL] | | | | | | |
| AUC/Dose | 158 | 199 | 533 | 498 | 1300 | 565 |
| [[hr*μg/ | | | | | | |
| mL]/[mg/ | | | | | | |
| kg]] | | | | | | |
| CL [mL/day/ | 152 | 120 | 45.0 | 48.2 | 18.4 | 42.5 |
| kg] | | | | | | |
| Vₛₛ [mL/kg] | 67.5 | 67.6 | 48.4 | 42.6 | 25.2 | 52.9 |
| T_{1/2} [d] | 0.35 | 0.39 | 0.53 | 0.51 | 1.10 | 0.88 |

It has been found that the PK can be well captured by a two-compartment model including target-mediated disposition and immunogenicity-related clearance (see FIG. 20). These are known for other molecules. [22,23]

In the limit of high doses, a linear clearance of 0.818 mL/(h*kg) was estimated, corresponding to a 3 to 4-times higher clearance than expected for human IgG antibodies in cynomolgus monkeys.[23] For the target-mediated disposition, rapid binding equilibrium and constant target pool were assumed fixing the KD to in-vitro-measured CD20 value of 3.3 nM, resulting in an estimated binding capacity of ˜400 pmol/kg.[24]

### Pharmacokinetics in CSF

CSF samples were collected via the cisterna magna and lumbar puncture at different time points and across dose groups. For each animal and each time of collection, the ratio of RG6035 concentration in CSF measured via cisterna magna and via lumbar puncture was calculated as indicated in Table 7.

TABLE 7
Individual CSF concentrations of RG6035 after single
IV administration of RG6035 at 0.3 mg/kg, 1 mg/kg and
10 mg/kg in cynomolgus monkeys; BLQ: below the limit
of quantification; CSF: cerebrospinal fluid; CSF-CM:
CSF cisterna magna; CS-LP: CSF lumbar puncture.

| | | | | CSF | CSF | |
|---|---|---|---|---|---|---|
| | | | | cisterna | lumbar | |
| | | | | magna | puncture | Ratio |
| Dose | | | Time | (CSF-CM) | (CSF-LP) | CSF-CM/ |
| [mg/kg] | Group | Individual | [h] | [μg/mL] | [μg/mL] | CSF-LP |
| 0.3 | 3 | 3501 | 8 | 0.00760 | 0.0102 | 0.7 |
| 0.3 | 3 | 3502 | 24 | 0.00433 | 0.0126 | 0.3 |
| 0.3 | 3 | 3501 | 96 | 0.000278 | BLQ | — |
| 1 | 4 | 4501 | 8 | 0.0283 | 0.0284 | 1.0 |
| 1 | 4 | 4501 | 48 | 0.0249 | 0.0544 | 0.5 |
| 1 | 4 | 4501 | 96 | 0.00587 | 0.00636 | 0.9 |
| 1 | 4 | 4502 | 24 | 0.0243 | 0.0479 | 0.5 |
| 10 | 2 | 2501 | 8 | 0.373 | 0.339 | 1.1 |
| 10 | 2 | 2503 | 24 | 0.0993 | 0.476 | 0.2 |
| 10 | 5 | 5501 | 8 | 0.185 | 0.718 | 0.3 |
| 10 | 5 | 5501 | 96 | 0.0755 | 0.108 | 0.7 |
| 10 | 5 | 5501 | 336 | 0.00146 | BLQ | — |
| 10 | 5 | 5502 | 24 | 0.758 | 0.918 | 0.8 |
| 10 | 5 | 5502 | 48 | 0.420 | 0.726 | 0.6 |
| 10 | 5 | 5502 | 168 | 0.0108 | 0.0121 | 0.9 |

The ratio of RG6035 concentration in CSF sampled from cisterna magna versus lumbar puncture ranged from 0.2 to 1.1. PK parameters from CSF cisterna magna sampling were calculated based on composite profile per dose group. Similar to serum, exposure to RG6035 in CSF increased slightly more than dose-proportionally with area under the curve from zero to infinity (AUC_{0_inf}) of 0.241, 2.19 and 35.7 h*μg/mL for dose groups 0.3 mg/kg, 1 mg/kg and 10 mg/kg, respectively. The maximum concentration observed (Cₘₐₓ) also increased more than dose-proportionally with Cₘₐₓ of 0.0076, 0.0283 and 1.06 μg/mL for dose groups 0.3 mg/kg, 1 mg/kg and 10 mg/kg, respectively. CSF concentration amounted for 0.13% to 3.4% of serum concentration.

It has been found that a model consisting of a single compartment for each CSF compartment with inflow proportional to the serum concentration and first-order outflow captures the data well (FIG. 21).

Equilibrium CSF-to-serum concentration ratios of 0.00537 and 0.00628 are estimated for lumbar and cisternal CSF, respectively. The first-order outflow rate is assumed to be equal for the two compartments (and brain tissues) with an estimated half-life of ˜16 hours.

### Pharmacokinetics in Brain Regions

RG6035 concentrations in different brain regions and tissues were measured at necropsy on Day 3 (Group 2) or Day 6 (Group 6) following IV administration of RG6035 at 10 mg/kg. Using an exploratory approach, based on results for the blood tracer in serum and brain tissues at necropsy, RG6035 concentrations in the different brain regions were corrected for blood contamination. The results for the blood tracer in serum and brain tissues indicated that blood tracer concentration was two orders of magnitude higher in serum than in any of the brain tissues. This demonstrated efficient removal of the blood from most brain regions during the brain perfusions at necropsy. It has been found that a model consisting of a single compartment for each tissue compartment with inflow proportional to the serum concentration and first-order outflow captures the data well.

RG6035 uptake into brain vascular endothelial cells and parenchyma was confirmed, consistent with the expected TfR1-mediated transcytosis across the BBB. The highest RG6035 concentrations were detected in the meninges while in all other brain tissues RG6035 concentrations were comparable. The brain AUC exposure amounted for 1% (cortex) and 0.8-1.5% across different brain regions (excluding meninges) and CSF of serum exposure (AUC), corresponding to a ˜25-fold higher brain AUC (2.5 to 60-fold across different brain regions and CSF) at steady state compared with a typical IgG, such as obinutuzumab.

It has been found that TfR1 binding by RG6035 is expected to be similar in cynomolgus monkeys and humans in vivo. Therefore, no major species differences in net brain uptake are anticipated. The current inventions is based, at least in part, on the assumption that the found increased brain uptake in cynomolgus monkeys is translates to the clinical situation.

### Pharmacodynamics of B-cells in Blood

RG6035 induced a rapid dose-dependent decrease in peripheral B-cells as early as 4 hours post-dose. Four phases of the PD response of blood B-cells to RG6035 exposure were observed: an initial rapid drop (up to 85% depletion, without obvious dose dependency), followed by a rebound (3 to 7 days post-dose), after which a phase of dose-dependent sustained depletion (˜2 weeks post-dose) occurred, with B-cell counts eventually returning to baseline. Similar decreases were also observed in all B-cell subsets (naïve, non-switched memory, and CD27+-switched memory B-cells) and appeared to be more pronounced and sustained in the high-dose group and in the memory populations (unswitched and CD27+-switched B-cells).

The current inventions is based, at least in part, on the finding that a PK/PD model combining concentration-driven B-cell depletion and redistribution phenomena between two exchanging B-cell pools captures the B-cell dynamics well (see FIG. 22). In this model, observed B-cells are part of the blood pool, which is receiving B-cells from a hidden pool of tissue-resident B-cells.

With this model, a steady concentration of 0.0758 μg/mL is estimated to be sufficient to deplete B-cells by 50%. The half-life of the pool of tissue-resident B-cells is estimated to be ˜18 days, in line with published observations.[25-28] It has been found, according to this model, that the manifestation of the B-cell depleting effect of RG6035 in blood is delayed with respect to the depletion in the main pool. The half-life of this delay is ˜3 days for memory B-cells and ˜8 days for naïve B-cells, resulting in different PD profiles for these cell types.

A short-lived dose-independent initial depletion with a half-life of 1.1 days adds to the complexity of the PD. Without being bound by this theory, it is assumed that this is caused by a re-distribution phenomenon but is of no major pharmacological importance. It has been found that the parameter estimation of this model is mostly robust.

### Pharmacodynamics in Peripheral Tissue

RG6035 induced a decrease in the relative percentages of B-cells in the spleen, mandibular lymph nodes and tonsils 48 hours post-dose and in the mesenteric lymph nodes 120 hours post-dose (FIGS. 23 and 24), without an apparent effect on total T cell and natural killer (NK) cell relative percentages.

### Effective and Long-Lasting B-Cell Depletion in Surrogate Organs

Staining patterns were similar for the four tissues assessed (spleen, tonsil, cervical, and mandibular lymph node). Tonsils were missing for one animal in Group 2. Cervical lymph nodes were missing for one animal in both Group 1 and Group 6. Mandibular lymph node samples from Group 2 were not further analyzed due to a highly unspecific background. Staining patterns for CD19 and CD20 on consecutive sections were qualitatively very similar (FIG. 23). Both markers labeled total B-cell population: for further staining, an anti-CD19 antibody was used to identify the B-cell population, which was found to be equivalent in consecutive sections of tonsil, cervical, and mandibular lymph nodes for all treatment groups. RG6035-treated animals showed a decrease in the percentage of CD19+-B-cells as well as per tissue area, in all investigated lymphoid tissues when compared to the vehicle-treated animals without further significant decrease between different time points (FIG. 23A-D; Table 8; FIG. 25).

TABLE 8
CD19+-B-cells of total cells across all organs and stainings.

| Tissue - | Vehicle 48 h | | | 10 mg/kg 48 h | | | 10 mg/kg 120 h | | |
|---|---|---|---|---|---|---|---|---|---|
| Staining | Mean | SD | n | Mean | SD | n | Mean | SD | n |
| Cervical | 26.969 | 0 | 1 | 1.312 | 1.61 | 2 | 0.138 | 0.19 | 2 |
| LN - | | | | | | | | | |
| Staining 1 | | | | | | | | | |
| Cervical | 28.985 | 0 | 1 | 0.448 | 0 | 1 | 0.006 | 0 | 1 |
| LN - | | | | | | | | | |
| Staining 2 | | | | | | | | | |
| Tonsil - | 29.882 | 7.39 | 2 | 1.497 | 0 | 1 | 8.000 | 6.15 | 2 |
| Staining 1 | | | | | | | | | |
| Tonsil - | 25.799 | 16.27 | 2 | 0.287 | 0 | 1 | 7.807 | 2.94 | 2 |
| Staining 2 | | | | | | | | | |
| Mandibular | 24.511 | 11.34 | 2 | na | na | na | 0.146 | 0.13 | 2 |
| LN - | | | | | | | | | |
| Staining 1 | | | | | | | | | |
| Mandibular | 10.860 | 14.32 | 2 | 0.202 | 0 | 1 | 0.123 | 0.05 | 2 |
| LN - | | | | | | | | | |
| Staining 2 | | | | | | | | | |
| Spleen - | 14.008 | 9.73 | 2 | 4.399 | 1.37 | 2 | 0.871 | 0.28 | 2 |
| Staining 1 | | | | | | | | | |
| Spleen - | 5.926 | 2.04 | 2 | 1.606 | 0.82 | 2 | 0.367 | 0.02 | 2 |
| Staining 2 | | | | | | | | | |

### Human Projection

The invention is based, at least in part, on the finding that a projection of the PK/PD to humans has the perspective of reaching ≥95% of B-cell depletion after 4 weeks. FIG. 26 presents simulations of PK, brain uptake, blood B-cells and CSF B-cells with four monthly subcutaneous doses of 150 mg of RG6035.

Blood B-cells show a gradual accumulating decline during the dosing and repletion after dosing stops. The dynamics in CSF integrates both i) a reduction of B-cells in the brain due to the reduced influx from blood and ii) the local depletion, enhanced by the TfR1-mediated transport of RG6035 to the brain tissue and by the direct local cell killing. Without being bound by this theory, it is assumed that the effect of the direct cell killing should lead to a deeper and more rapid B-cell depletion in CSF than a purely passive depletion driven by the blood depletion alone.

The examples, sequences and figures are provided to aid the understanding of the present invention, the true scope of which is set forth in the appended claims. It is understood that modifications can be made in the procedures set forth without departing from the spirit of the invention.

### CITATIONS

All documents cited herein are expressly incorporated by reference herein.
1. Compston, A. and Coles, A., The Lancet 372 (2008) 1502-1517.
2. Reich, D. S., et al., Multiple Sclerosis. 378 (2018) 169-180.
2a. Niewoehner, J., et al., Neuron 81 (2014) 49-60.
3. Wallin, M. T., et al., Neurol. 92 (2019) e1029-e1040.
4. Cencioni, M. T., et al., Nat. Rev. Neurol. 17 (2021) 399-414.
5. Lee, D. S. W., et al., Nat. Rev. Drug Discov. 20 (2021) 179-199.
6. Obermeier, B., et al., Nat. Med. 14 (2008) 688-693.
7. Serafini, B., et al., Brain Pathol. 14 (2004) 164-174.
8. Magliozzi, R., et al., Brain 130 (2007) 1089-1104.
8a. Wallin, M. T., et al., Neurol. 92 (2019) e1029-e1040.
9. Lucchinetti, C. F., New Engl. J. Med. 365 (2011) 2188-2197.
9a. Furuzawa-Carballeda, J., et al., Autoimmun. Rev. 6 (2007) 169-175.
10. Obermeier, B., et al., Nat. Med. 19 (2013) 1584-1596.
10a. Sospedra, M. and Martin, R., Annu. Rev. Immunol. 23 (2005) 683-747.
11. Abbott, N.J., et al., Neurobiol. Dis. 37 (2010) 13-25.
11a. Chisari, C. G., et al., J. Neurol., 8 (2021) 1-25.
12. Daneman, R. and Prat, A., Cold Spring Harbor perspectives in biology 7 (2015) a020412.
12a. Gasperi, C., et al., Dis. Manag. 6 (2016) 37-47.
13. Freskgård, P. O. and Urich, E., Neuropharmacol. 120 (2017) 38-55.
13a Gelfand, J. M., et al., Neurotherap. 14 (2017) 835-841.
14. Johnsen, K. B., et al., Prog. Neurobiol. 181 (2019) 101665.
14a. Greenfield, A. L. and Hauser, S. L., Ann. Neurol. 83 (2018) 13-26.
15. Klein, C., et al., MAbs 5 (2013) 22-33.
15a. Jakimovski, D., et al., Expert Opin. Biol. Ther. 17 (2017) 1163-1172.
16. Al-Sawaf, O., et al., Drug Des. Devel. Ther. 11 (2017) 295-304.
16a. Montalban, X., et al., N. Eng. J. Med. 376 (2017) 209-220.
17. Herter, S., et al., Mol. Canc. Ther. 12 (2013) 2031-2042.
17a. Sabatino, J. J. Jr., et al., Cold Spring Harb. Perspect. Med. 9 (2019) a032037.
18. Kern, D. J., et al., Leukem. Lymph. 54 (2013) 2500-2505.
18a. Howell, O. W., et al., Brain 134 (2011) 2755-2771.
19. Lim, S. H., et al., Blood 118 (2011) 2530-2540.
19a. Reali, C., et al., Brain Pathol. 30 (2020) 779-793.
20. Honeychurch, J., et al., Blood 119 (2012) 3523-3533.
20a. Florou, D., et al., Brain Sci. 10 (2020) 758.
21. Niederfellner, G., et al., Blood 118 (2011) 358-367.
22. Dirks, N. L. and Meibohm, B., Clin. Pharmacokinet. 49 (2010) 633-659.
22a. Klein, C., et al., mAbs 5 (2013) 22-33.
23. Wang, J., et al., Biopharm. Drug Dispos. 37 (2016) 51-65.
23a. Tobinai, K., et al., Adv. Ther. 34 (2017) 324-356.
24. Gibiansky, L., et al., J. Pharmacokinet. Pharmacodyn. 35 (2008) 573-591.
24a. Herter, S., et al., Haematologica 103 (2018) e78-e81.
25. Macallan, D. C., et al., Blood 105 (2005) 3633-3640.
25a. Couch, J, A, et al., Sci. Transl. Med. 5 (2013) 183ra157.
26. De Boer, R. J., et al., J. Immunol. 170 (2003) 2479-2487.
27. Pan, S., et al., Br. J. Clin. Pharmacol. 85 (′2019) 1790-1797.
27a. Weber, F., et al., Cell Rep. 22 (2018) 149-162.
28. Grimm, H. P., et al., J. Pharm. Sci. 108 (2019) 3729-3736.
28a. Bessa, J., ez al., Pharm. Res. 32 (2015) 2344-2359.
29. Howell, O. W., et al., Brain 134 (2011) 2755-2771.
29a. Leon, L. R., Front. Biosci. 9 (2004) 1877-1888.
30. Bell, L., et al., Front. Immunol. 10 (2020) 3090.
30a. Candelaria, P. V., et al., Front. Immunol. 12 (2021) 607-692.
31. Reali, C., et al., Brain Pathol. 30 (2020) 779-793.
31a. Neiveyans, M., et al., mAbs 11 (2019) 593-605.
32. Bittner, S. and Wiendl, H., Neurotherapeut. 13 (2016) 4-19.
33. Sabatino, J. J., et al., Proc. Natl. Acad. Sci. 116 (2019) 25800-25807.
33a. Palanichamy, A., et al., Sci. Transl. Med. 6 (2014) 248ra106-248ra106.
34. Brell, J., et al., Commun. Biol. 3 (2020) 621.
34a. van Meer, P. J. K., et al., mAbs 5 (2013) 810-816.
35. Trenor, C. C., 3rd, et al., Blood 96 (2000) 1113-1118.
35a. Merchant, A. M., et al., Nat. Biotechnol. 16 (1998) 677-681.
36. Jabara, H. H., et al., Nat. Gen. 48 (2016) 74-78.
36a. Klein, C., et al., mAbs 8 (2016) 1010-1020.
37. Pulgar, V. M., Front. Neurosci. 12 (2019) 1019-1019.
37a. Schaefer, W., et al., Proc. Natl. Acad. Sci. 108 (2011) 11187.
38. Schumacher, V., et al., Unpublished results.
38a. Schlothauer, t., et al., Protein Eng. Des. Sel. 29 (2016) 457-466.
39. Bell, L., et al., Histochem. Cell Biol. 151 (2019) 489-499.
40. Sefia, E., et al., Mult. Sclerosis Rel. Disord. 14 (2017) 46-50.
40a. Clavarino, G., et al., PLOS One 11 (2016) e0162209.
41. Anthony, I. C., et al., Brain 126 (Pt 5, 2003) 1058-1067.
41a. Gong, Q., et al., J. Immunol. 174 (2005) 817.
42. Bischoff, T., et al., Cytomet. A 79 (2011) 436-445.
43. Pieper, K., et al., J. Allergy Clin. Immunol. 131 (2013) 959-971.
44. Cross, A., et al., Neurol. 92 (15 Supplement, 2019) S56.008.
45. Syková, E., Neurochem. Int. 45 (2004) 453-466.
46. Comi, G., et al., Ann. Neurol. 89 (2021) 13-23.
47. Bjornevik, K., et al., Science 375 (2022) 296-301.
48. Moessner, E., et al., Blood 3 (2010) 4393-4402, 17.

### DESCRIPTION OF THE FIGURES

FIG. 1: Projected B-cell depletion based on multiple subcutaneous doses (once every week for four weeks).

FIG. 2: Binding of RG6035, obinutuzumab-PGLALA and rituximab to CD20 as measured by flow cytometry, using a human B-cell lymphoma cell line. A fluorescein isothiocyanate-labeled F(ab)′2 goat anti-human Fcγ-specific antibody was used for detection, and the median fluorescence intensity (MFI) was measured.

FIG. 3: Percentage of Annexin V and PI double positive B-cell lymphoma cells as determined by flow cytometry after 24 h incubation with different concentrations (0.01 nM to 1000 nM) of RG6035, obinutuzumab-PGLALA or rituximab.

FIG. 4: Human TfR-mediated internalization of RG6035, obinutuzumab-PGLALA and BS-DP47-PGLALA in MDCKII-TfR and MDCKII parental cells.

FIG. 5: Time-course of transcytosis efficiency: comparison between BS-obinutuzumab, RG6035 and BS-DP47-PGLALA

FIG. 6: B-cell depletion in response to treatment with mBS-obinutuzumab mAbs by FACS analysis; the frequency of remaining B220+-B-cells in blood was calculated by setting the percentage of B220+-B-cells at the baseline measurement to 100%; data points represent mean±SD blood B-cell depletion measured in 3 groups of 5 mice: does was 0.6 mg/kg.

FIG. 7: B-cell depletion in response to treatment with mBS-obinutuzumab mAbs by FACS analysis; the frequency of remaining B220+-B-cells in blood was calculated by setting the percentage of B220+-B-cells at the baseline measurement to 100%; data points represent mean±SD blood B-cell depletion measured in 3 groups of 5 mice: does was 1.3 mg/kg.

FIG. 8: B-cell depletion in response to treatment with mBS-obinutuzumab mAbs by FACS analysis; the frequency of remaining B220+-B-cells in blood was calculated by setting the percentage of B220+-B-cells at the baseline measurement to 100%; data points represent mean±SD blood B-cell depletion measured in 3 groups of 5 mice: does was 13.3 mg/kg.

FIG. 9: B-cell depletion in response to treatment with mBS-obinutuzumab antibodies by FACS analysis; percentage of remaining B220+-B-cells in lymph node were calculated by setting the frequencies of B220+-B-cells in vehicle-treated animals (N=5) as 100%; data represents mean±SD measurements from N=5 mice per group; day 3.

FIG. 10: B-cell depletion in response to treatment with mBS-obinutuzumab antibodies by FACS analysis; percentage of remaining B220+-B-cells in lymph node were calculated by setting the frequencies of B220+-B-cells in vehicle-treated animals (N=5) as 100%; data represents mean±SD measurements from N=5 mice per group; day 8.

FIG. 11: B-cell depletion in response to treatment with mBS-obinutuzumab antibodies by FACS analysis; percentage of remaining B220+-B-cells in lymph node were calculated by setting the frequencies of B220+-B-cells in vehicle-treated animals (N=5) as 100%; data represents mean±SD measurements from N=5 mice per group; day 22.

FIG. 12: B-cell depletion in response to treatment with mBS-obinutuzumab antibodies by FACS analysis; percentage of remaining B220+-B-cells in spleen were calculated by setting the frequencies of B220+-B-cells in vehicle-treated animals (N=5) as 100%; data represents mean±SD measurements from N=5 mice per group; day 3.

FIG. 13: B-cell depletion in response to treatment with mBS-obinutuzumab antibodies by FACS analysis; percentage of remaining B220+-B-cells in spleen were calculated by setting the frequencies of B220+-B-cells in vehicle-treated animals (N=5) as 100%; data represents mean±SD measurements from N=5 mice per group; day 8.

FIG. 14: B-cell depletion in response to treatment with mBS-obinutuzumab antibodies by FACS analysis; percentage of remaining B220+-B-cells in spleen were calculated by setting the frequencies of B220+-B-cells in vehicle-treated animals (N=5) as 100%; data represents mean±SD measurements from N=5 mice per group; day 22.

FIG. 15: B-cell depletion following antibody administration in cynomolgus monkeys; B-cell numbers following antibody administration are presented as a percentage of B-cells measured at baseline (100%); data represents mean measurements from N=4 animals.

FIG. 16: Percentages of positive cell types in cervical lymph nodes normalized to tissue area. Bars marked with “*” represent p<0.05 compared to vehicle control.

FIG. 17: Percentages of positive cell types in spleen normalized to tissue area. Bars marked with “*” represent p<0.05 compared to vehicle control.

FIG. 18: Percentages of positive cell types in mandibular lymph nodes normalized to tissue area. Bars marked with “*” represent p<0.05 compared to vehicle control.

FIG. 19: Percentages of positive cell types in tonsils normalized to tissue area. Bars marked with “*” represent p<0.05 compared to vehicle control.

FIG. 20: PK modeling in cynomolgus monkeys using target-mediated drug disposition and additional time-dependent clearance to capture the effect of immunogenicity; observations are shown as circles above 1E−4, observations below the limit of quantification are shown at 1E−1, individual model simulations as lines; overlays are shown for selected animals, at 0.3 mg/kg (top row), 1 mg/kg (middle row), and 10 mg/kg (bottom row).

FIG. 21: Brain distribution of RG6035 in cynomolgus monkeys is modeled as a single compartment with uptake from circulating compound in blood and with a first-order outflow; observations are shown as circles above 1E−4, observations below the limit of quantification as circles at 1E−4, individual model simulations as lines; overlays are shown for selected animals for lumbar CSF, at 0.3 mg/kg (top row), 1 mg/kg (middle row), and 10 mg/kg (bottom row).

FIG. 22: PK/PD modeling of the effect of RG6035 on blood B-cells in cynomolgus monkeys; observations are shown as circles above 1E1, observations below the limit of quantification as circles at 1E1, individual model simulations as lines, and individual baseline estimates as dashed teal lines; overlays are shown for selected animals switched memory B-cells, at 0.3 mg/kg (top row), 1 mg/kg (middle row), and 10 mg/kg (bottom row).

FIG. 23: Effective and long-lasting B-cell subset depletion in lymphoid organs; A: representative overview image (A-C) and insert (a-c) of spleen and corresponding mandibular lymph node in vehicle (A, a) and RG6035-treated animals after 48 h (B, b) and 120 h (C, c) stained with anti-CD19 antibody (green), revealing a strong decrease in B-cells; DAPI (blue) shows the nuclei.

FIG. 24: Quantification of B-cells across lymphoid organs by cell segmentation and subsequent thresholding for CD19+-cells.

FIG. 25: Representative images of equivalence of CD19 (green; A, a) and CD20 (ref; B, b) in tonsils, and nuclei staining (DAPI, blue); staining with anti-CD20 and anti-CD19 antibody on consecutive sections revealed very similar staining patterns in all investigated lymphoid tissues; thus, for the B-cell subset, characterization of CD19 was used to identify the B-cell population.

FIG. 26: Human PK/PD projection for RG6035 following 4 monthly doses of 150 mg subcutaneously (SC); right lower panel: B-cells in CSF, effect of TfR-mediated brain uptake and direct B-cell killing of RG6035 (straight line) and passive/no active transport from reduced migration of B-cells from the blood to the brain (dashed line).

FIG. 27: Based on the DAPI channel (A, a), nuclei segmentation is performed (B, b) including defining cytoplasm and membrane compartment around the nuclei (1 μm around the nucleus); thresholding for CD19 (green; C, c) was adjusted based on minimum intensity in a vehicle animal found in cytoplasm/membrane compartment, resulting in a CD19+ cell depicted in yellow (D, d).

FIG. 28: Structure of the full model used for PK/PD projections for RG6035 in human; the PK (purple boxes) is governed by a two-compartment model with target-mediated disposition, represented by the central (serum) and the peripheral compartments; optionally, absorption from subcutaneous injection can be modeled as a first-order process; rain uptake is described by a linear distribution process; the distribution within the brain tissue is assumed to be confined to the interstitial space; B-cells (blue boxes) exchange between the blood and a ‘hidden pool’ (“tissues”); in absence of pharmacological intervention, the latter is maintained in balance between proliferation and loss; RG6035 adds to the B-cell loss by cell killing from the “tissues” pool; B-cells in the brain either derive from blood or from local proliferation and are lost either by returning to blood or by cell death; a local cell killing effect is driven by the brain interstitial concentration of RG6035; PD: pharmacodynamic; PK: pharmacokinetic.

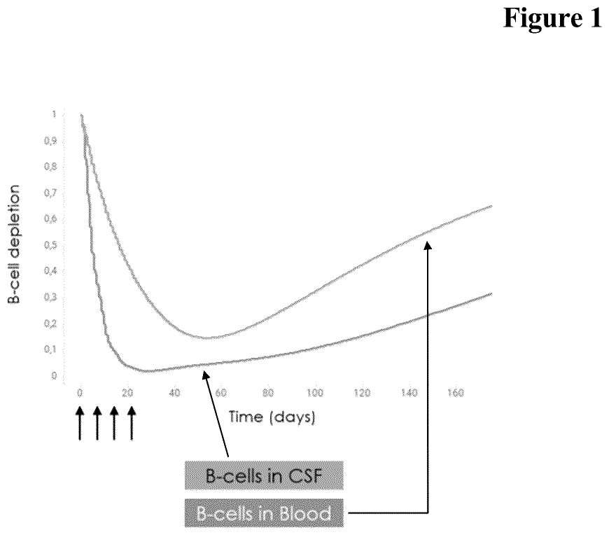

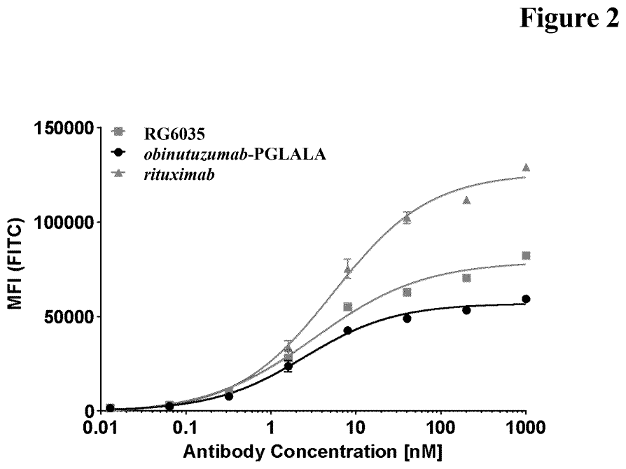

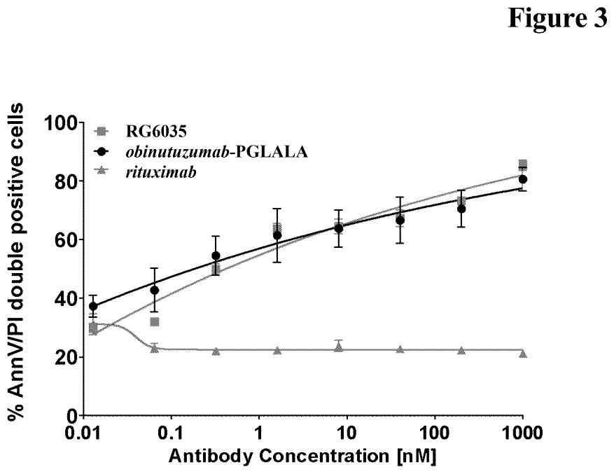

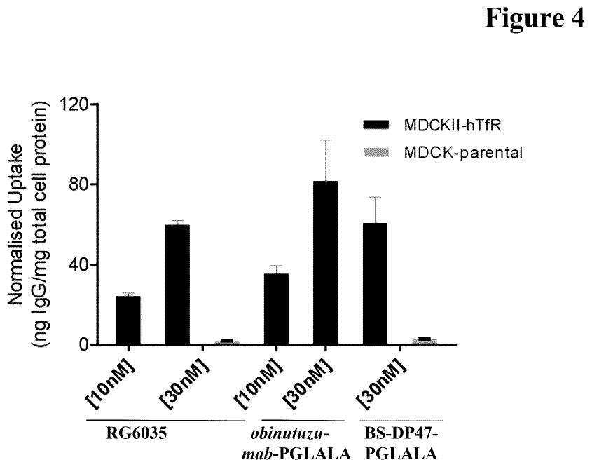

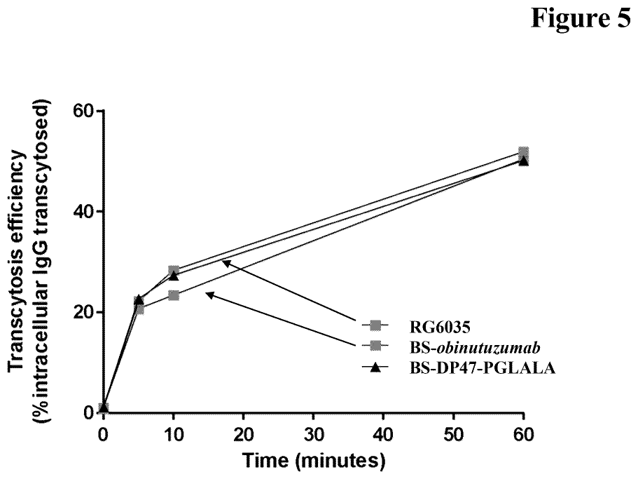

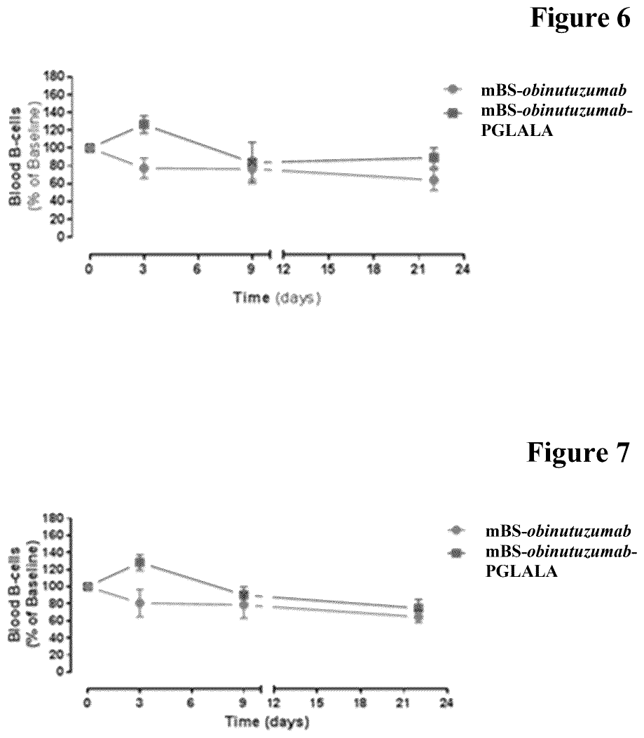

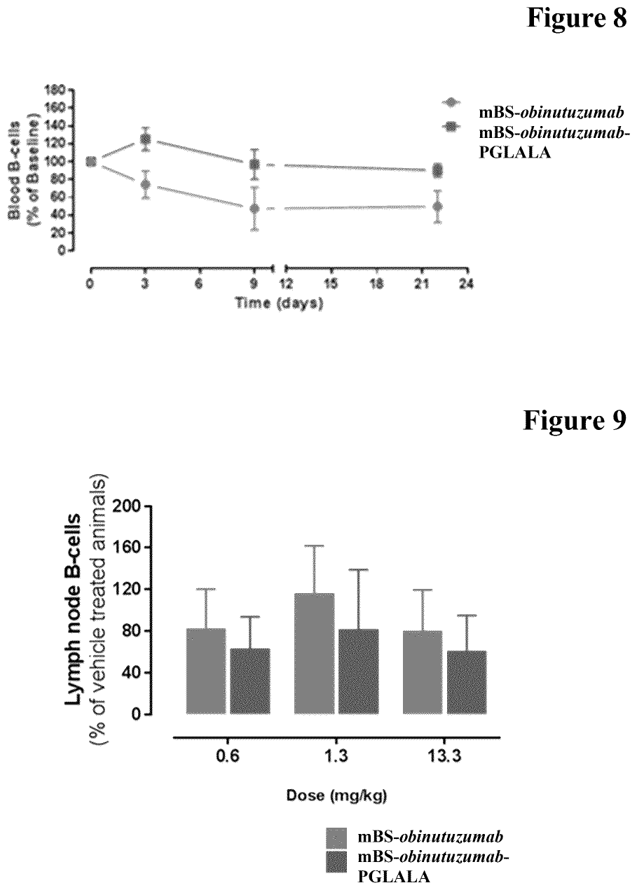

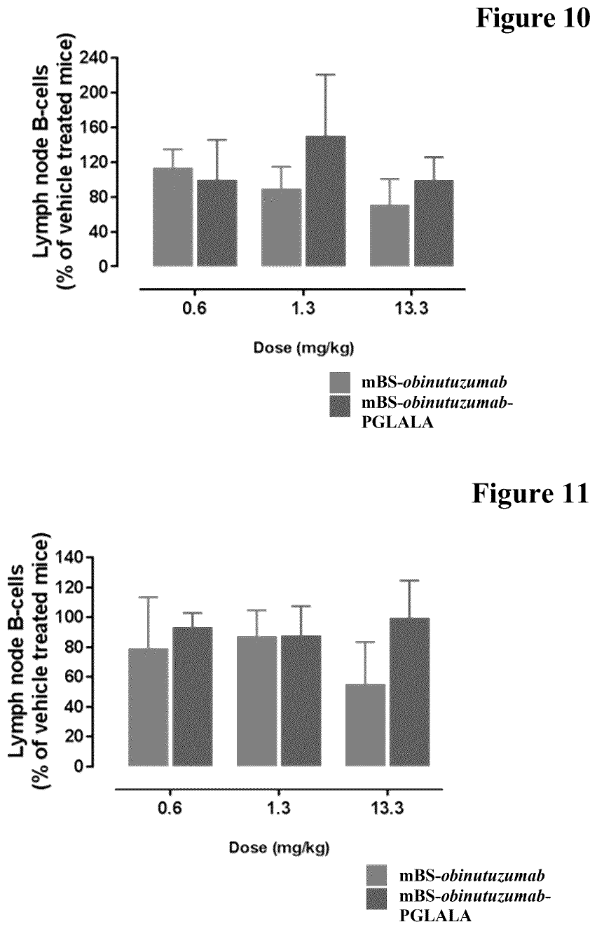

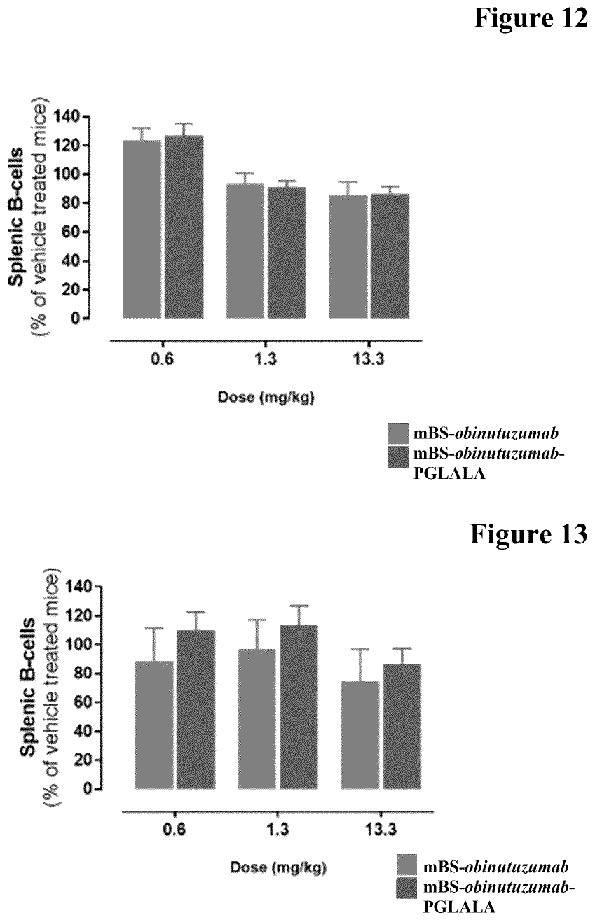

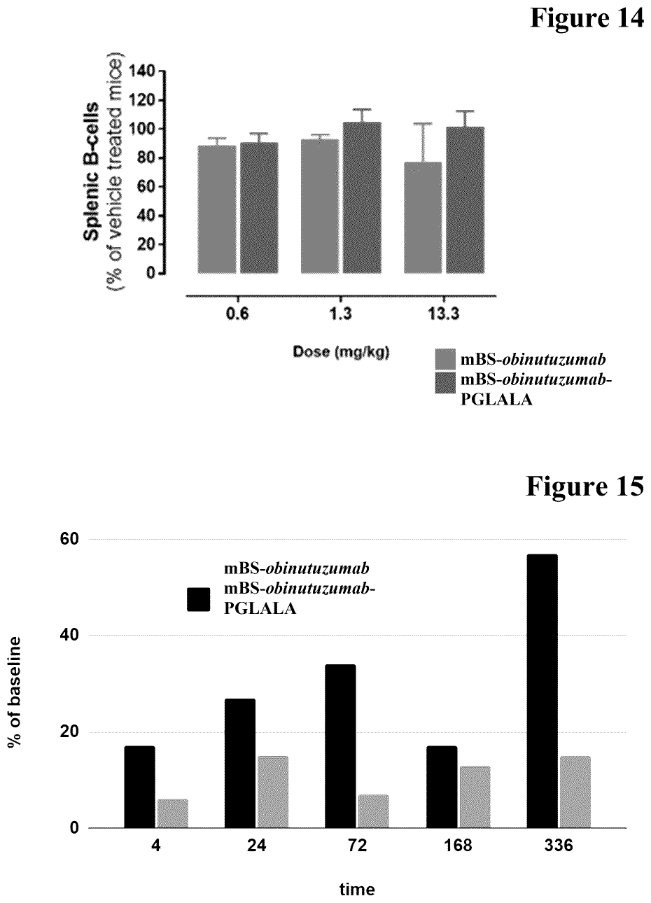

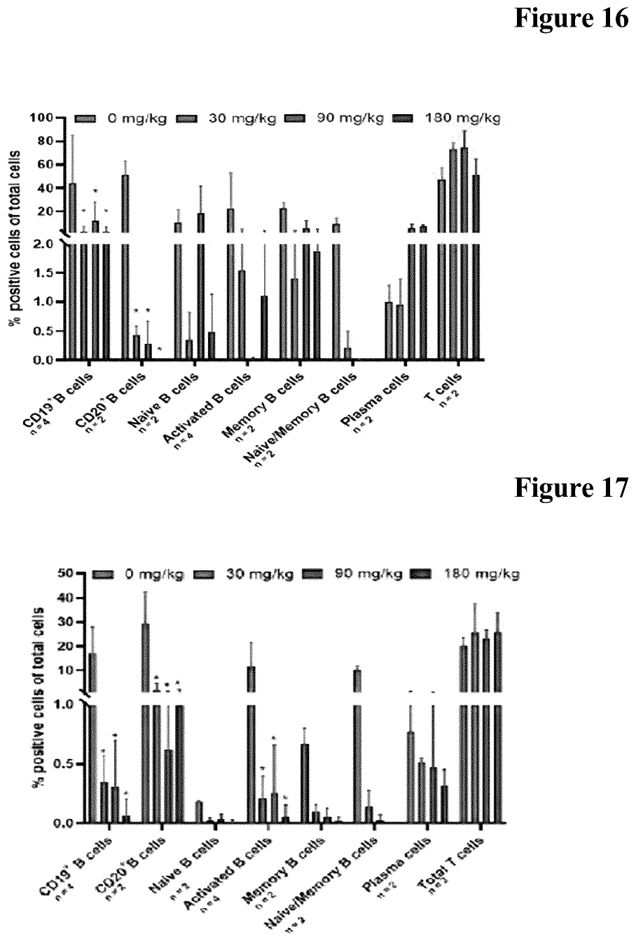

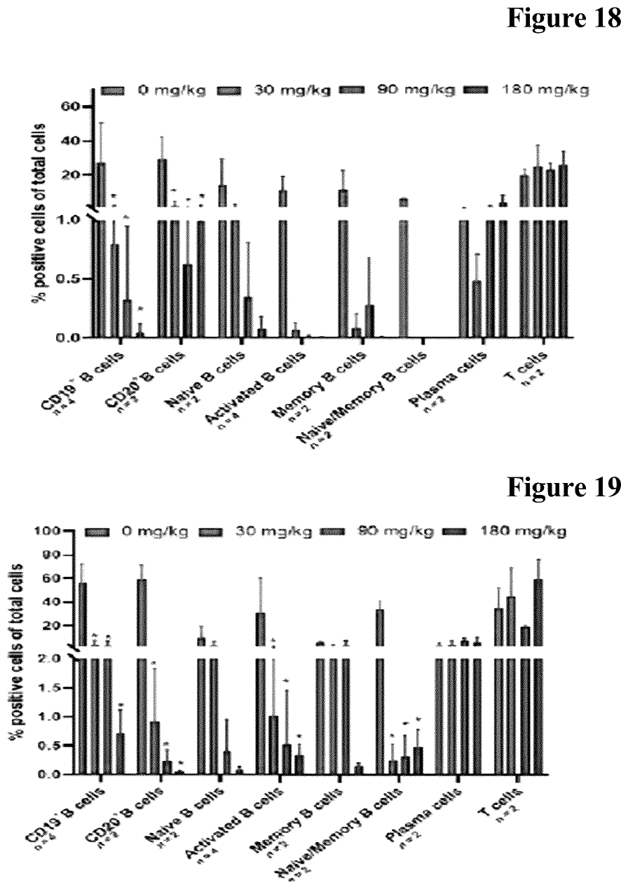

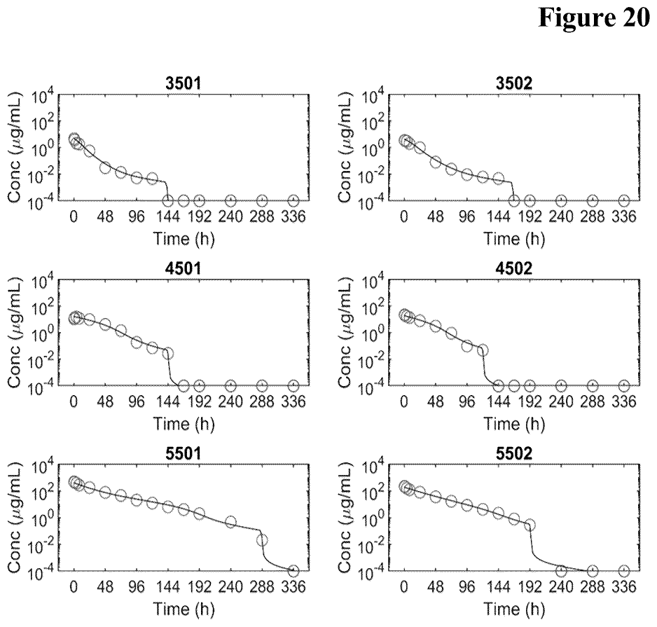

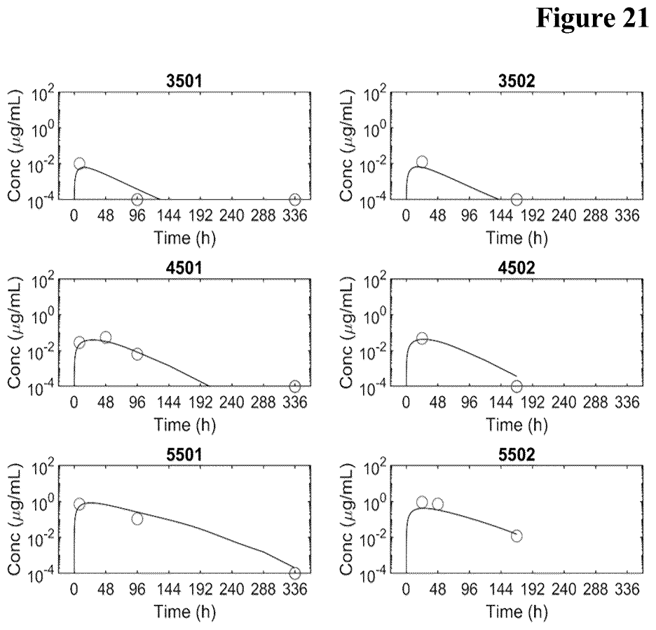

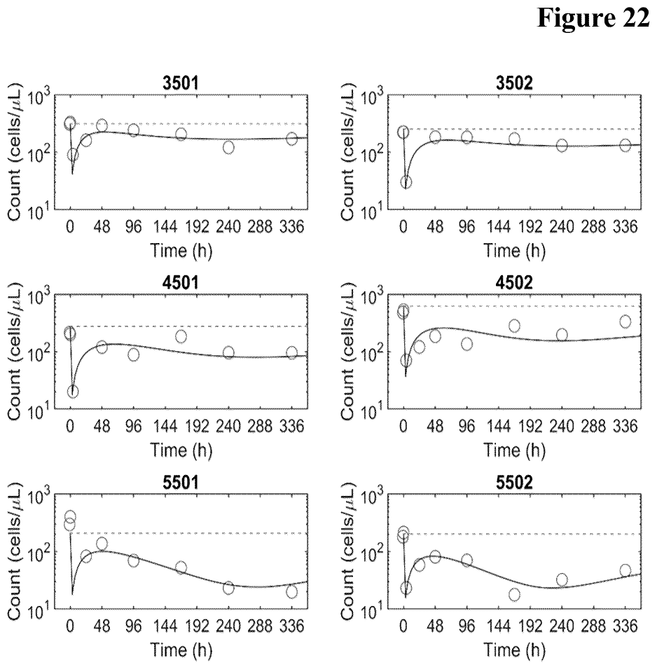

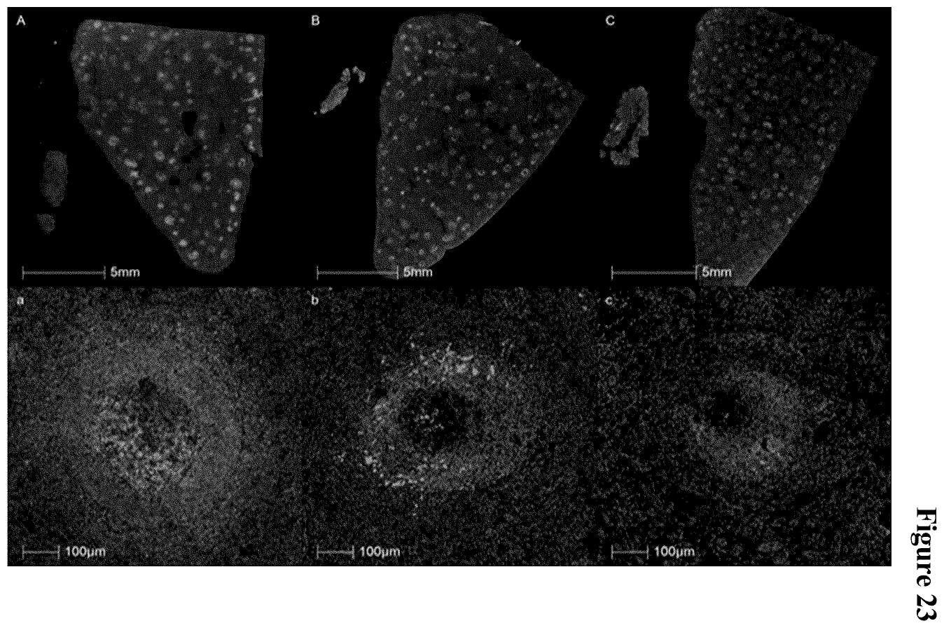

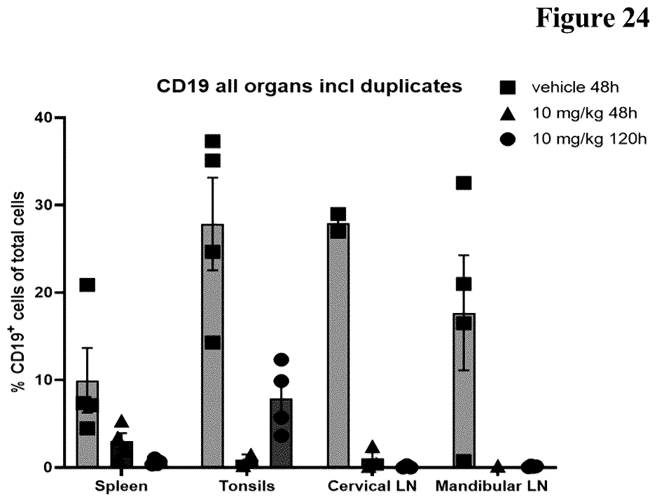

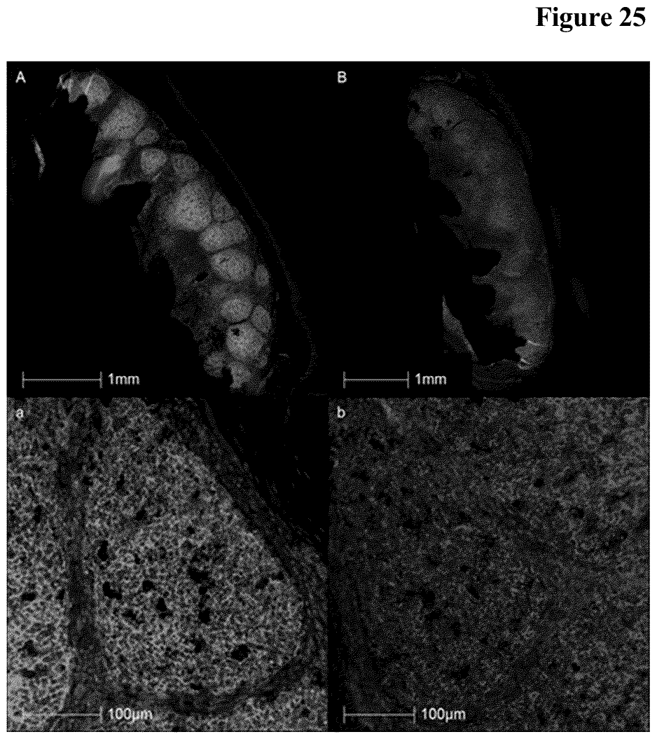

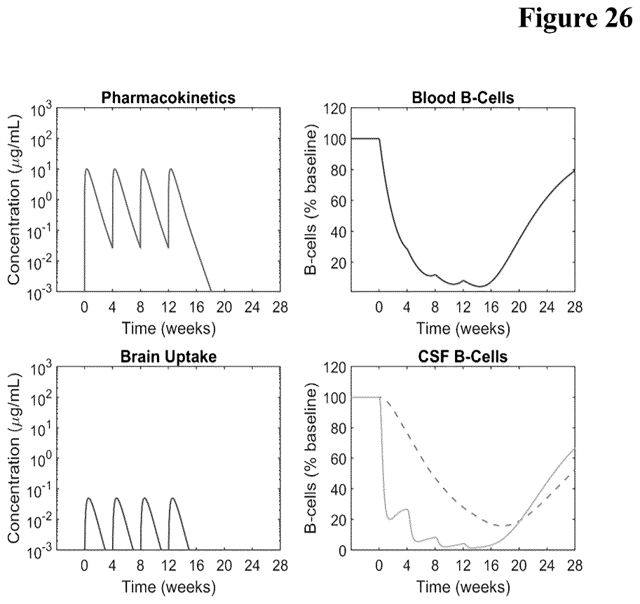

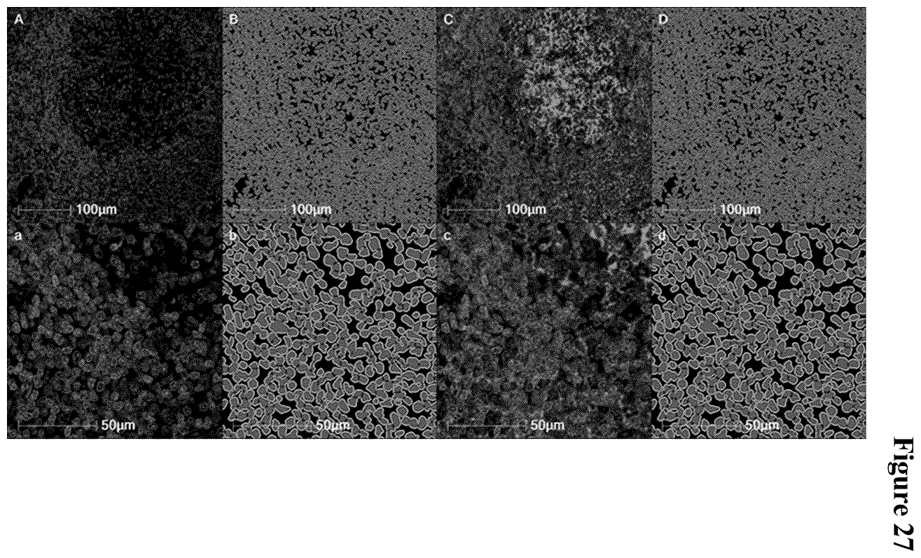

### DESCRIPTION OF THE SEQUENCES

SEQ ID NO: 01 RG6035 light chain 1.
SEQ ID NO: 02 RG6035 heavy chain 1.
SEQ ID NO: 03 RG6035 light chain 2.
SEQ ID NO: 04 RG6035 FAB fragment
SEQ ID NO: 05 RG6035 heavy chain 2
SEQ ID NO: 06 human CD20.

### Experimental Section

To show the advantageous safety and efficacy properties of RG6035, safety and efficacy profiles of BS and control antibodies were directly compared in preclinical models.

### Antibody and Bispecific Antibody Production

BS constructs were engineered by fusing an anti-huTfR1 cross Fab (human; clone 1026) to the C-terminus of a heavy chain of obinutuzumab or obinutuzumab-PGLALA variant. [36a, 38a] Knobs-into-holes technology [35a] was used to favor heterodimeric pairing of a heavy chain carrying the BS module with a non-fused heavy chain. CrossMab technology [36a, 37a] was used to avoid mispairing of the different light chains on the antibody complex. In the same way, the huTfR1 positive control reference binder, BS-DP47, was generated, except that the fusion partner was DP47 (a non-target binding germline human IgG used as a blood tracer for brain contamination), instead of anti-CD20. To generate the mouse TfR1 surrogate BS, an anti-muTfR1 single chain Fab (rat; clone 8D3) was fused to the C-terminus of the heavy chain of obinutuzumab or obinutuzumab-PGLALA variant, also utilizing knobs-into-holes technology. A glycine-serine peptide linker was used to construct the single-chain Fab fragment.

Genes for the different chains of the different antibodies were cloned into expression cassettes, which harbor regulatory elements for the expression of the genes comprising CMV promoter, bGH poly A signal, and hGH transcriptional terminator. The expression cassettes were cloned into expression vectors. Expi293™ or ExpiCHO-S™ cells (ThermoFisher Scientific; A14527 and A29127, respectively) were co-transfected with the appropriate vector combination according to the manufacturer's protocol and antibodies were purified from cell culture supernatants as previously described [2a].

### CD20 Binding

Cells were incubated with test antibodies and FACS analysis was performed to assess CD20 binding.

Human B-cell lymphoma cells were maintained in RPMI1640 medium (ThermoFisher, 11875093) with 10% fetal calf serum (FCS; Gibco 16140) and 1% (v/v) 2 mM N-acetyl-L-alanyl-L-glutamine (GlutaMAX™, ThermoFisher, 35050061) at 0.3 to 0.9*1E6 cells/mL at 37° C. in a 5% CO₂ humidified incubator. Harvested cells (98.2% viability) were pelleted by centrifugation (4 min., 400×g) and re-suspended in Fluorescence-Activated Cell Sorting (FACS) buffer (phosphate buffered saline (PBS) with 2% (v/v) FCS, 5 nM EDTA, and 0.25% sodium azide) at about 0.6*1E6 viable cells/mL and seeded into 96-U-bottom plates at 1*1E5 cells per well. Test antibodies were added to cells with final concentrations ranging from 1000 nM to 0.0128 nM (1:5 dilution steps, in triplicate) for 30 min at 4° C. Cells were then washed with FACS buffer and incubated with a secondary antibody (Jackson ImmunoResearch, 109-096-098; FITC F(ab)′2 anti-human Fcg specific; 1:20) for 30 min at 4° C. Cells were then washed twice with FACS buffer and fixed using 2% paraformaldehyde in FACS buffer before proceeding with FACS acquisition (BD FACS CantoII flow cytometer). FACS gating was performed using FACS Diva Software and the median fluorescence intensities were determined. EC50 values were calculated based on sigmoidal dose-response (variable slope) analysis using GraphPad Prism. No further statistical analysis was performed.

### Cell Death Induction

Human B-cell lymphoma cells were incubated with test antibodies (0.0128 nM to 1000 nM) for 24 hours before measuring cell death by assessing Annexin V (annV) expression and propidium iodide (PI) uptake.

Human B-cell lymphoma cells were seeded in a 96-U-bottom plate at 1*1E5 cells per well. Test antibodies (0.0128 nM to 1000 nM in triplicate) were incubated with cells at 37° C. for 24 hours. Afterwards, cells were resuspended and centrifuged (4 minutes at 400×g), then washed with annV Binding Buffer (BB; 10 mM HEPES/NaOH pH 7.4, 140 mM NaCl, 2.5 mM CaCl₂). Final cell pellets were resuspended in annV-FLUOS (Roche Diagnostics GmbH, Mannheim, Germany; 11828681001; 1:100 in annV BB) before a 15-minute incubation at room temperature with protection from light. Cells were then washed with annV BB before the addition of annV BB containing PI (Merck, P4864; 1:4000). Signal acquisition was performed immediately upon addition of PI using a BD FACS CantoII flow cytometer. FACS gating was performed using BD FACS Diva Software to determine the percentages of annV/PI double positive cells. No further statistical analysis was performed.

### TfR-Mediated Cell Internalization and Transcytosis

Parental MDCKII cells were cultured in high-glucose DMEM (ThermoFisher, 41965062) supplemented with 10% (v/v) FCS (Sigma-Aldrich, F4135) at 37° C. in a 5% CO₂ humidified incubator. Cells were seeded into 24-well plates at 7.5*1E4 cells per well and transiently transfected 24 hours post seeding. For MDCKII transient transfection, vector DNA mix was prepared in Opti-MEM™ reduced serum media (ThermoFisher, 31985070) at a concentration of 0.02 μg/μL, giving 0.5 μg DNA per 25 μL volume transferred per well (i.e., the total amount of DNA per 24-well plate well for transfection). Lipofectamine™ 2000 reagent (ThermoFisher, 11668027) was also diluted in Opti-MEM reduced serum media, to a concentration giving 1.5 μL Lipofectamine™ per 25 μL transferred per well. The separately prepared diluted DNA and Lipofectamine™ reagent were combined in a 1:1 ratio and incubated at room temperature for 10 minutes to allow formation of DNA/Lipofectamine™ complexes. After incubation, transfection complexes were carefully transferred into each well. Parallel mock transfections were included, where the same procedure was conducted for transfection but without the vector DNA.

MDCKII cells were transiently transfected with huTfR 24 hours prior to the transcytosis assays. huTfR-mediated internalization (uptake) and transcytosis (pulse-chase) assays were performed 24-hours post transfection; each treatment condition was evaluated in triplicate. The assay buffer (AB) used consisted of Hanks balance salt solution (Mg++/Ca++; ThermoFisher, 14065072) supplemented with 20 mM HEPES (ThermoFisher, #15630056), pH 7.4. All assay incubation steps were conducted at 37° C., 5% CO₂ without shaking. Cells were rinsed twice with AB prior to initiation of the pulse phase (uptake/cell loading) with the addition 0.2 mL AB spiked with test antibodies per well. Antibodies were incubated with the cells for the indicated time points (30 or 45 minutes), depending on the experiment. At the end of the incubation, uptake was stopped with the immediate aspiration of spiked AB, followed by two rapid rinses with AB containing 0.1% (v/v) bovine serum albumin (BSA), followed by a final wash with AB without BSA to remove any residual protein. The pulse step was performed in duplicate plates; one plate was immediately lysed, representing the intracellular compartment IgG content at the start of the chase phase; time 0, and the other plate evaluated across multiple time points. Following a final rinse step (as described above), cells were incubated with pre-warmed AB (37° C.) and left to initiate the chase. Samples of the AB (extracellular compartment) were then collected at the end of each time point (5, 10, and 30 minutes). At the end of the final time point, cells were rinsed three times as described above for the pulse phase, before being lysed and assayed for intracellular IgG content.

To measure intracellular IgG content, cells were lysed in RIPA Lysis and Extraction Buffer (ThermoFisher Scientific, 89900) containing protease inhibitors (Sigma Aldrich, 11697498001) and incubated for 30 minutes at 4° C. (under frequent agitation). Solubilized cell lysates were transferred to 96-well LoBind deep well plates and stored at 4° C. (or −80° C. for longer periods) until ready for analysis. Samples from the chase phase were immediately diluted with assay buffer in 96-well LoBind deep well plates. Total cell protein content in cell lysates were determined using the Pierce™ BCA Protein assay (ThermoFisher, 23225), following the manufacturers' protocol. IgG content was quantitatively evaluated using generic IgG ELISA with chemiluminescence-based detection.

### B-Cell Depletion in Human CSE

Human PBMCs and human CSF were used as a matrix for B-cell depletion assays. PBMCs from five healthy donors and CSF from 12 individual donors (female and male) across all age groups were obtained from Discovery Life Sciences, Huntsville, USA. CSF samples were thawed once, pooled, aliquoted, and stored at −80° C. until needed. PBMCs were thawed in RPMI (with 10% (v/v) FCS, 1% penicillin/streptomycin (PS), and 1% non-essential amino acid (NEAA)) and equilibrated at 37° C., 5% CO₂ for at least 2 hours. Approximately 2*1E6 cells/mL (2*1E5 per well) were seeded in a 96-well plate. After 2 hours of incubation, the plate was centrifuged at 300×g for 5 minutes at room temperature. Cells were then resuspended in 100 μL human CSF.

Treatment antibodies were diluted and added to PBMCs in CSF in the range of 0.002 nM to 100 nM for 22 hours. Cells were then pelleted and processed for FACS staining with the following antibodies: anti-CD45 antibody (APC, F20; BD Biosciences, 560973), anti-CD19 antibody (PE, F20; BioLegend, 982402), and anti-CD3 antibody (PE/Cy7, F20; BioLegend, 300419). Cell viability was measured with the LIVE/DEAD™ Fixable Aqua Dead Cell Stain Kit (ThermoFisher, L34957), Zombie Aqua™ Fixable Viability Kit (BioLegend, 423101), and F1000 BioLegend. Samples were measured with a BD Biosciences FACSLyric™ flow cytometer using FACS Suite software in a volume-based acquisition. FACS raw data was analyzed using FlowJo software. B-cell depletion was calculated using the B-cell/T-cell ratio of untreated control versus the B-cell/T-cell ratio of the sample. Statistical evaluations were obtained using GraphPad Prism. Statistical significance of the difference in maximal CD19+-B-cell depletion between the different antibodies was calculated using a one-way analysis of variance (multiple comparisons).

### B-Cell Depletion in Human Tonsil-Derived Cells

Freshly resected tonsil tissue was obtained from adult donors undergoing routine tonsillectomy at Hirslanden Clinic Muenchenstein Birshof, Switzerland. Written patient consent was obtained for each patient-derived sample. Upon collection, tissue was stored in 4° C. in Hanks balanced salt solution (Mg++/Ca++; ThermoFisher, 14065-072). Subsequently, tonsil tissue underwent mechanical and enzymatic digestion to obtain a cell suspension, which was directly used for B-cell depletion assays. Concentration-dependent and B cell subset depletion was determined as described below.

### Total B-Cell Depletion

B-cells were transferred to 96-well U bottom plates in cultivation medium (RPMI with 10% FBS, 1% PS, and 1% NEAA at 2.5*1E5 cells/well). Treatment antibodies were diluted and added in the range of 0.03 nM to 100 nM. Further experimental conditions follow B-cell depletion in human CSF (described above).

### B-Cell Subset Depletion

B-cells were transferred to 96-well U bottom plates in cultivation medium (1*1E6 cells/well). Treatment antibodies were diluted and added at 1 nM to 100 nM and incubated for 8 hours at 37° C., 5% CO₂. Cells were then pelleted and processed for FACS staining. Cell viability was measured with the LIVE/DEAD™ and Zombie Aqua™ kits, the antibodies listed in Table 9, and BD Horizon™ Brilliant Stain Buffer (BD Biosciences, 563794). Samples were analyzed on a FACSymphony™ or FACS LSRFortessa™ using FACSDiva™ software (BD Biosciences). The phenotypic identification of B-cell subsets was performed based on the surface marker expression and staining strategy previously described [40a].

TABLE 9
Antibodies used for B-cell depletion and human whole blood assay.

| | | Company |
|---|---|---|
| Antibody | Clone | (Product Code) |
| B cell subset depletion | | |
| FITC Mouse Anti-Human IgM | MHM-88 | BioLegend (314506) |
| PE Mouse Anti-Human IgG | G18-145 | BD Biosciences |
| | | (560951) |
| BUV395 Mouse Anti-Human | HB15e | BD Biosciences |
| CD83 | | (740311) |
| PE-Cy ™7 Mouse Anti-Human | O323 | ThermoFisher |
| CD27 | | Scientific |
| | | (25-0279-42) |
| APC-H7 Mouse Anti-Human CD10 | HI10a | BD Biosciences |
| | | (655404) |
| BV786 Mouse Anti-Human CD19 | SJ25C1 | BD Biosciences |
| | | (563326) |
| Human WBA | | |
| PerCP-Cy ™5.5 Mouse Anti- | UCHT1 | BD Biosciences |
| Human CD3 | | (560835) |
| FITC Mouse Anti-Human CD14 | M5E2 | BD Biosciences |
| | | (557153) |
| V450 Mouse Anti-Human CD4 | RPA-T4 | BD Biosciences |
| | | (560345) |
| APC-Cy ™7 Mouse Anti-Human | SK1 | BD Biosciences |
| CD8 | | (348793) |
| PE-Cy ™7 Mouse Anti-Human | HIB19 | BD Biosciences |
| CD19 | | (560728) |
| V500 Mouse Anti-Human CD45 | HI30 | BD Biosciences |
| | | (560777) |
| CD56 APC | NCAM16.2 | BD Biosciences |
| | | (341027) |
| PE Mouse Anti-Human CD20 PE | L27 | BD Biosciences |
| | | (347201) |
| Control antibodies/solutions | | |
| Erbitux ® (Anti-EGFR Rabbit | 11E19 | Merck (ZRB04338) |
| Monoclonal Chimeric IgG1) | | |
| Lemtrada ® (Anti-CD52, | | Sanofi |
| Humanized Rat Monoclonal IgG1) | | Genzyme[Me- |
| | | ridian1] |
| Lipopolysaccharide | | Merck (L5886) |
| Phosphate Buffered Saline | | ThermoFisher |
| | | Scientific |
| | | (14190094) |

Lymphocytes were identified based on size (forward scatter and granularity side scatter), as well as single-cell gating. Following a live-dead discrimination, live CD45+-lymphocytes were further gated into CD3−- and CD3+-cells. CD3+-T-cells were later used to normalize for B-cell subset population depletion. CD19 positivity was used to identify total B-cells of interest. CD19+-B-cell subsets were further identified based on surface marker expression. Thereby, distinct expression of CD38 and CD10 on CD19+-B-cells served as initial discriminator of three major B-cell populations.

Plasma cells express the highest levels of CD38 and do not express CD10 (CD38+++/CD10−), whereas CD38-low and CD10-CD19+-B-cells give rise to CD27+-memory and CD27−-naive B-cells. CD38-intermediate (CD38++) and CD10+CD19+-B-cells can be further broken down to GC B-cells, transitional B-cells, and immature B-cells. During transition from immature bone marrow-derived B-cells to peripheral blood transitional cells, CD5 expression is upregulated. Accordingly, CD38++CD10+CD5+-cells have been classified as transitional B-cells. Furthermore, immature B-cells have been discriminated from GC B-cells based on their surface IgM expression. IgD-CD38++CD10+-cells were considered as GC B-cells and IgD− but IgM+CD38++CD10+-cells as immature B-cells.

### Human Whole Blood Assay (WBA)

Fresh, undiluted human blood samples from six healthy individuals were incubated for 24 hours with concentrations of 0.01, 0.1, 1.0, 10, 100, 500 and 1000 nM of test antibodies and control antibodies (Table 9). Subsequently, cytokine release (IL-1β; IL-6; IL-8; TNF-α and IFN-γ) and B-cell depletion (only for the concentrations 0.01, 1.0, 100, and 1000 nM) were measured.

Venous blood from six healthy donors was collected in vacutainer tubes containing lithium heparin as anticoagulant (Roche medical Center, Basel, Switzerland) and kept at room temperature until initiation of the assay (within 1 to 3 hours to avoid erythrocyte lysis). Antibodies were incubated with blood samples in U-bottom 96-well plates (1:40); antibody concentrations ranged from 0.01 to 1000 nM (see above) for RG6035, BS-obinutuzumab, obinutuzumab, obinutuzumab-PGLALA, and for the negative comparator Erbitux® (final concentration 100 μg/ml) and the positive comparator Lemtrada® (final concentration 100 μg/ml). The 1000 nM compound concentration tested covers predicted human Cₘₐₓ concentrations (maximum serum concentration). These conditions ensured optimal performance with respect to practicality and efficiency to gain at least 70 μL of plasma and sufficient cells to conduct multi-cytokine analysis. Endogenous activation of blood cells and responsiveness was assessed by including controls containing phosphate-buffered saline, vehicle, or lipopolysaccharide. After incubation for 24 hours at 37° C., cells and plasma were separated by centrifugation at 1,800×g for 5 minutes. Plasma samples were stored at −80° C. until analysis of cytokine content. Pre-tests revealed that cytokine levels did not differ between fresh and thawed samples. Determination of cytokine concentrations was performed on diluted (1:5) plasma samples. Analyte concentrations were determined by enzyme-linked immunosorbent assays (ELISAs) using multiplex cytokine chemiluminescent assay kits (Ciraplex™ Chemiluminescent Array Kit; Aushon BioSystems, 101-3EF-1-AB) with the SignaturePLUS™ imaging system and the PROarray analysis software (Aushon BioSystems).

For data points indicated as “smaller than” (<) the maximum indicated value was taken. For data points indicated as “bigger than” (>) the minimum indicated value was taken. Data are presented as the mean cytokine measurements in supernatants from triplicate wells. Cell staining for flow cytometry was performed 24 hours following the incubation period in cell culture medium containing individual test items or controls. Cell suspensions were incubated with the antibodies of interest for 30 minutes at room temperature and washed twice with PBS (phosphate buffered saline solution) to remove any non-bound antibodies. Cell samples were then analyzed on a BD Biosciences FACSCantoII flow cytometer. Flow cytometry data was analyzed using FlowJo.2, Microsoft Excel, and Graph Pad Prism 7.

### Animal Care and Handling

Study plans and any amendments or procedures involving the care and use of animals in all experiments was reviewed and approved by the Institutional Animal Care and Use Committee of the respective institutions. All mouse studies were performed by F. Hoffmann-La Roche AG (Basel, Switzerland). Pharma Research and Early Development Basel Facilities are fully accredited by the Association for Assessment and Accreditation of Laboratory Animal Care International (AAALAC), and all procedures were in accordance with the respective Swiss regulations and approved by the Cantonal Ethical Committee for Animal Research. Cynomolgus monkey studies were performed at contract research organizations in Germany and the United Kingdom, sponsored by F. Hoffmann-La Roche AG; all procedures were compliant with the German Animal Welfare Act or the EU Directive 2010/63/EU, respectively.

### Transgenic Mice

Transgenic mouse models are based on the C57BL/6 background. C57BL/6 humanized CD20 mice (huCD20) were generated as the result of a random integration of a human CD20 bacterial artificial chromosome transgene as previously described [41a]. C57BL/6-Tg (hIg-γ1,κ,λ)ait mice (HIGR3) were generated as previously described [28a]. Three transgenic constructs were used for co-injection into pronuclei; these constructs contained unrearranged miniloci of the human Ig heavy chain γ1, the human Ig light chains K, and 2, respectively. The transgenic mice inherited these transgenes at a single locus and subsequently express human IgG1 antibodies in their blood plasma [28a]. The C57BL/6 huCD20xC57BL/6-Tg (hIg-γ1,κ,λ)ait mice (huCD20xHIGR3) are the result of crossing the huCD20 and HIGR3 mouse lines; these mice are transgenic for the human CD20 drug target and do not generate ADAs to human antibodies.

### Preserved Potency of Fc-Silent BS-CD20

Humanized CD20 transgenic mice (huCD20) were treated via IV administration with murine BS-obinutuzumab (mBS-obinutuzumab) at 0.6, 1.3, and 13.3 mg/kg (mBS-obinutuzumab, or mBS-obinutuzumab-PGLALA), or 0.5, 1, and 10 mg/kg (equimolar concentrations) of parental (non-shuttled) obinutuzumab (obinutuzumab or obinutuzumab-PGLALA). At days-1, 2 and 6 blood samples were taken for determination of frequencies of B-cells (B220+-B-cells). At day 6, mice were sacrificed and the frequencies of B220+-B-cells were determined in spleen and inguinal draining lymph node (LN). Spleens and inguinal LN from 6 naïve mice (i.e. untreated mice) were harvested to set the baseline B-cell frequencies.

### Flow Cytometry Immunization Efficacy and Potency Studies

Absolute total B-cell counts were determined using Trucount™ beads (BD Biosciences, 340335). Fc receptors were blocked with anti-mouse CD16/CD32 antibodies (BioLegend 101320). Cells were sorted with an LSRFortessa™ cytometer (BD Biosciences) and analyzed using FlowJo software (BD Biosciences).

### IRR and Tolerability Studies

A one-day tolerability study was conducted in huCD20xHIGR3 mice. Five groups of two male and two female mice/group were administered 0 (vehicle control), 3, or 10 mg/kg mBS-obinutuzumab-PGLALA or mBS-obinutuzumab as a single IV bolus (5 mL/kg). Assessment of toxicity was based upon mortality and clinical observations on the day of administration. Cytokine release and exposure assessment were evaluated in terminal retro-orbital blood samples collected approximately 2 hours post administration. IL-2, IL-6, IL-10, MCP-1, MIP-1β, MIP-2, G-CSF, IFN-γ, TNF-α, and KC serum cytokines were measured via Luminex assays (R&D Systems).

To determine the reason behind any differences in the MTD toxicity profile and whether the effects were dependent on Fc-region activity and/or the BS moiety, mBS-obinutuzumab, mBS-obinutuzumab-PGLALA, and obinutuzumab were assessed in a single IV dose study in huCD20 or huCD20xHIGR3 transgenic mice. The acute phase response was characterized by assessing cytokine levels (IL-2, MIP-2, IL-6, G-CSF, IL-10, IFN-γ, MCP-1, TNF-α, MIP-1β and KC, measured in serum 2 hours from terminal retro-orbital blood collection via Luminex assays) and body temperature (measured via telemetry; BMDS IPTT300 system (Plexx)) to identify the potential to generate IRRs. Cytokine release and exposure assessment were evaluated in terminal retro-orbital blood samples collected approximately 2 hours post administration.

### PK and PD Studies

Mouse Single-Dose PK Study in huCD20xHIGR3 Mice

mBS-obinutuzumab and mBS-obinutuzumab-PGLALA PK were assessed after single-dose IV bolus administration (13.3 mg/kg) in 12 female huCD20xHIGR3 mice. Serum sampling was performed until 168 hours post dosing (based on 8 time points in serum: 1, 3, 7, 24, 48, 72, 96, 168 hours post dose (composite, 3 samples per animal)) and terminal brain sampling collection was performed at 24, 48, or 168 hours post dosing. Blood samples (0.05 mL) were collected from tail vein and heart puncture for terminal sampling time, respectively. DP47 (human IgG blood tracer for brain contamination) was administered via IV injection (30 mg/kg) 5 minutes prior to animal sacrifice. PK parameters were derived from composite concentration data and were estimated by non-compartmental analysis using the kinetic evaluation program Phoenix@ Version 1.4.

Serum and brain blood tracer levels were measured, and serum and brain samples were analyzed using two different ELISAs. For the CD20-specific ELISA method (bioanalytical method 1), capture antibody (anti-IgG mAb M-6.28.530-IgG-Bi), positive control standards, or diluted samples and the detection antibody (anti-IgG mAb M.1.19.31-IgG-Dig) were successively added to streptavidin-coated microtiter plate (SA-MTP). Immobilized immune complexes were detected with a polyclonal anti-digoxygenin-horseradish peroxidase (POD) conjugate (Merck, 11633716001). Finally, the formed immobilized immune complexes were visualized by addition of ABTS™ solution (Merck, 11684302001) —a POD substrate. Color change intensity was analyzed photometrically (BioTek MTP Reader ELx808™) at 405 nm (490 nm reference wavelength) and was proportional to analyte concentrations in test samples. Before necropsy, animals were treated with blood tracer intravenously at 30 mg/kg, 5 minutes before sacrifice. Necropsy samples (serum and brain lysates) were analyzed using an ELISA method described below. Prior to analysis, tissue samples were mechanically lysed in 800 μl of Tissue Extraction Reagent I (ThermoFisher, FNN0071) containing protease inhibitors (Merck, P8340) using the MagNA Lyser Instrument (Roche Diagnostics GmbH, Mannheim, Germany).

For the blood tracer-specific ELISA method (bioanalytical method 2), capture antibody (mAb 2B01-6D01-Bi), diluted calibrators, as well as diluted quality controls and samples, detection reagent (anti-IgG mAb M1.7.24-IgG-Dig), and anti-digoxygenin-POD were added successively to SA-MTPs. The formed immobilized immune complexes were visualized by addition of ABTS™ solution. Color change intensity was analyzed photometrically, as described above for bioanalytical method 1. Absorbance quantification was performed using a calibration curve with a non-linear, four parameter Wiemer-Rodbard curve fitting function. Analytical sensitivity was 7.8 ng/mL in 100% serum and brain lysate with method 1, and 11 ng/ml in 100% serum and brain lysate with method 2.

For the mouse PK study, no formal statistical analysis was performed due to the composite study design. The data are summarized as arithmetic means only (or medians for time to peak drug concentration).

PK/PD Study in huCD20xHIGR3 Mice

One control group (0 mg/kg) and six groups of 15 female huCD20xHIGR3 mice were treated with mBS-obinutuzumab or mBS-obinutuzumab-PGLALA at 0.6, 1.3, and 13.3 mg/kg via single IV slow bolus administration (5 mL/kg). The control group was dosed with vehicle only; a solution containing 20 mM histidine and 140 mM NaCl at pH 6.0. Five animals per group were sacrificed post dosing at 48 hours (Day 3), 168 hours (Day 8), and 504 hours (Day 22).

Exposure assessment, mortality, clinical observations, body weight, hematology, cytokine (IL-6) level determination, inguinal lymph node and spleen organ weights, macroscopic findings, and histopathology of the inguinal lymph node, bone marrow and spleen were subsequently evaluated. FACS analysis was performed in blood, spleen, and inguinal lymph node to assess the efficacy of both BS-obinutuzumab constructs in depleting systemic and lymph node resident B-cells (CD19+B220+). Marginal zone (CD21+) and follicular B-cells were also assessed in the spleen. Approximately 50 μl blood per sampling time-point was collected from the tail vein for FACS analysis at pre-dose (Day 1; all 105 mice), 48 hours (Day 3; all 105 mice), 168 hours (Day 8; 70 mice), and 504 hours (Day 22; 35 mice). The following FACS antibody panel was assessed: anti-B220, anti-CD19, anti-huCD20, anti-TCRβ, anti-CD4, anti-CD8, anti-CD45, anti-CD21, anti-CD23, anti-IgM/IgD. Blood for hematology, exposure, ADA assessment, and IL-6 were terminally sampled retro-orbitally under anesthesia (ketamine/xylazine 150/9 mg/kg) shortly before exsanguination and necropsy on Day 3, 8, or 22. One sample of blood was collected into EDTA tubes to measure hematologic parameters (RBCs, WBCs, hemoglobin, hematocrit, mean corpuscular volume, mean corpuscular hemoglobin, mean corpuscular hemoglobin concentration, RBC distribution width, neutrophils, eosinophils, basophils, monocytes, lymphocytes, reticulocytes, platelets and peripheral blood smears). A 20 μl serum sample was used to determine IL-6 levels.

Two 50 μl serum samples were also collected for exposure and ADA assessment. Samples were analyzed by ELISA for the parent substances. Inguinal lymph node, bone marrow, and spleen were collected, formalin fixed, and embedded in paraffin. Slides were prepared and stained with hematoxylin and eosin, examined by the study toxicological pathologist and a peer reviewing toxicological pathologist under light microscopy. Samples of spleen were sectioned and stained with Prussian blue iron stain and assessed for percent area iron positivity with image analysis using HALO v1.2. For Prussian blue iron staining and assessment of iron positive percentage area using image analysis with HALO v1.2, the first step of the analysis consisted of identifying the spleen tissue versus non-tissue using a random forest classifier. The identified spleen tissue was defined as region of interest (ROI). The bright-field area quantification module was used to define Prussian blue positive areas within the ROI using real time tuning to apply thresholding parameters. Area was reported as μm²/tissue and as percentage positive stain/tissue.

### Cynomolgus Monkey Single-Dose Studies

BS-obinutuzumab and RG6035, respectively, were administered as a single IV bolus dose (10 mg/kg) to four female cynomolgus monkeys each, followed by a 15-day/8-week observation period, respectively. Clinical observations, body weight and body weight change, body temperature, clinical pathology (hematology, coagulation, and clinical chemistry), immunophenotyping, and cytokine evaluation were evaluated throughout the observation period, and organ weights and macroscopic/microscopic findings were assessed for RG6035. Blood hepcidin levels were assessed in the BS-obinutuzumab study only. For RG6035, blood samples were collected for hematology (0.5 mL in EDTA), clinical chemistry (2.0 mL), coagulation (1.0 mL trisodium citrate) and soluble transferrin (0.2 ml) once during the pre-dose phase, 24 hours post dosing, and on the day of necropsy. Blood was collected for cytokine analysis twice during the pre-dose phase and on Day 1 at 1 and 4 hours post dosing.

Cytokine levels were analyzed using Luminex multiplex kits (R&D Systems; IFNγ, TNFα, MCP-1, IL-6, IL-8). For immunophenotyping analysis on Day 1 (pre-dose) and at 4, 24, 72, 168, and 336 hours after dosing, blood samples (600 μL) were withdrawn from the vena cephalica antebrachii into EDTA anticoagulant. Immunophenotyping with specific monoclonal antibodies comprised of T-cells (T-helper-cells and cytotoxic T-cells), B-cells, monocytes, and NK cells.

Total lymphocyte counts were determined on the same day as the analysis of relative cell numbers. Absolute numbers of the lymphocyte subpopulations were determined from relative and total numbers. For the BS-obinutuzumab study, blood samples were collected for hematology (0.5 mL EDTA), coagulation (0.9 mL trisodium citrate), clinical chemistry (1.5 mL lithium heparin), and hepcidin (0.4 mL). Blood was collected from the femoral vein for cytokine analysis at pre-treatment and 2, 24, 72 hours post-dosing, and on Days 8 and 49 (0.3 mL, EDTA). Samples were assessed with a BioRad Bio-Plex 200 reader using a custom multiplex kit for IL-6, IL-8, IFNγ, TNFα, MCP-1. Blood was also collected from the femoral vein for flow cytometry at pre-treatment and 4-, 24-, and 73-hours post-dosing, and on Days 5, 6, 8, 11, 13, 15, 20, 23, 28, 30, 35, 37, 42, 44 and 49 (0.5 mL sodium heparin).

### Immunophenotyping and FACS Cynomolgus Monkey Studies:

FACS was performed to assess B-cell depletion using flow cytometry and other lymphocyte immunophenotyping assessments. The cellular antigens CD45 and CD19 for B-lymphocyte populations were quantified using specific antibodies against marker antigens and reported as absolute counts (cells/μL of blood). WBC counts (total and absolute differential) were determined from the whole blood sample using the ADVIA® 120 Hematology System (Siemens Healthineers). Total lymphocyte counts with antigen markers CD45+, CD14−, CD3+ and CD159α− were reported as cells/μL of blood and used for calculating absolute counts of the lymphocyte populations of interest. Change from baseline in B-cells was calculated for each animal as indicated in the following equation: percentage change in B-cells=absolute B-cell count at T(x) (hours)×100/absolute B-cell count at T(0) (hours). The SD from the mean of four animals was also calculated.

### Statistical Analysis:

For CD20 binding and direct B-cell death induction, FACS gating was performed using FACSDiva™ Software (BD Biosciences) and the median fluorescence intensities as well as the percentage of positive cells were determined. Half-maximal effective concentration values were calculated based on a sigmoidal dose-response (variable slope) analysis using GraphPad Prism. For IRR studies, temperature was measured in triplicate per animal and time point. Change in temperature was measured from pre-dosing temperatures.

### Cynomolgus Monkey PK/PD Study

This PK/PD study was conducted in cynomolgus monkeys to determine the pharmacokinetic properties of RG6035 as well as distribution of RG6035 in the brain, and to quantify B-cell depletion in blood and in the tissue of interest.

The study plan and procedures involving the care and use of animals in this study were reviewed and approved by CR MTL Institutional Animal Care and Use Committee (IACUC). During the study, the care and use of animals were conducted with guidance from the USA National Research Council and the Canadian Council on Animal Care (CCAC).

### Animals:

The cynomolgus monkey is the only cross-reactive species for the CD20- and TfR1-binding moieties of RG6035. Cynomolgus monkeys from Mauritius were provided by Research Models Houston (Texas, USA). Twelve females with a target age at initiation of dosing of 2-4 years and target weight of 2-3.5 kg were used in the study, two animals were kept as back-up. Before dosing initiation, all animals were weighed and socially housed in pens equipped with an automatic watering valve. Food was provided twice daily. Animals were assigned to one of six dosing groups using a computerized randomization procedure (Table 10). Each group consisted of two animals.

TABLE 10
Experimental design.

| | Dose | Dose | Dose | Number | |
|---|---|---|---|---|---|
| Group | Level | Volume | Concentration | of | Animal |
| No. | [mg/kg] | [mL/kg] | [mg/mL] | Females | Number |
| 1^{a} | 0 | 0.4 | 0 | 2 | 1501, 1602 |
| 2^{a} | 10 | 0.4 | 25 | 2 | 2501, 2503 |
| 3 | 0.3 | 0.4 | 0.75 | 2 | 3501, 3502 |
| 4 | 1 | 0.4 | 2.5 | 2 | 4501, 4502 |
| 5 | 10 | 0.4 | 25 | 2 | 5501, 5502 |
| 6 ^{b} | 10 | 0.4 | 25 | 2 | 6501, 6502 |

^{a}Animals in Group 1 and 2 were euthanized 48 hours post-dose after injection of blood tracer and underwent perfusion at necropsy;
^{b} Animals in Group 6 were euthanized 120 hours post-dose after injection of blood tracer and underwent perfusion at necropsy.

### Experimental Design:

Following the acclimation period of at least 4 weeks, animals underwent surgery for cannulation of the cisterna magna for CSF sampling as per Standard Operating Procedures (SOP). Each animal was pre-anesthetized with an intramuscular injection of glycopyrrolate, ketamine and xylazine to achieve sufficient sedation for presurgical preparation. During surgery, anesthesia was maintained using isoflurane. Catheter placement in the cisterna magna was confirmed by withdrawal of CSF during the surgical procedure.

RG6035 was administered as a single intravenous (IV) bolus at doses of 0.3 mg/kg (Group 3), 1 mg/kg (Group 4) and 10 mg/kg (Groups 2, 5, and 6) via an appropriate peripheral vein on Day 1. The control group (Group 1) received a single IV injection of the vehicle (20 mM histidine, 140 mM sodium chloride, pH 6.0).

For PK and PD analyses, blood and CSF samples were collected periodically from all animals. Animals assigned to Group 1, 2 and 6 received an IV injection of a blood tracer (see WO 2021/136772) at 10 mg/kg 15 minutes prior to their scheduled sacrifice; Groups 3, 4 and 5 were not scheduled for sacrifice. At necropsy on Day 3 (Groups 1 and 2) or Day 6 (Group 6), various tissues were harvested to assess the concentration of RG6035 and B-cell depletion in the brain, spleen, lymph nodes, tonsils, bone marrow, and liver.

### PK Sampling:

Blood samples were collected from each animal from an appropriate peripheral vein to assess B-cell depletion over time. Blood samples were mixed gently and kept under ambient conditions until centrifugation for 10 minutes at 2,400 g at 4° C. The resultant serum was separated, transferred to clear polypropylene tubes (target 2× 100 μL), and frozen (−80° C.) until further analysis.

PK CSF samples were collected over 2 weeks via lumbar puncture (target L5-L6 space, L4-L5 used if necessary) and from the cisterna magna catheter in all dosed groups. Animals were anesthetized with ketamine, dexmedetomidine and glycopyrrolate. Isoflurane was used as needed if sedation was deemed insufficient. 15 minutes before termination, the blood tracer was injected intravenously (saphenous or brachial vein) to Group 1, 2 and 6 animals, at a dose level of 10 mg/kg. 1 mL of blood was taken from a vein in the arm 10 minutes after injection of the blood tracer and serum was prepared.

Brain sampling with perfusion was performed across five brain regions in Groups 1, 2 and 6. Frozen 300 mg cynomolgus monkey brain tissue samples were thawed at room temperature for 2 hours. 800 μL of lysis buffer and one tablet of complete protease inhibitor cocktail (Roche Diagnostics GmbH, Mannheim, Germany) dissolved in 50 mL Tissue Extraction Reagent I (Invitrogen, Waltham, MA, USA) were added to the thawed brain tissue. The sample was homogenized for 20 seconds at 6,500 rpm and the tissue homogenate was then centrifuged for 10 minutes at 12,000 g. Finally, the supernatant was transferred to a 1.5 mL vial for further analysis or stored at −80° C.

### Quantification of RG6035 in CSF/Serum:

Test samples, calibration samples and quality controls were diluted to 1:100 in Roche Universal Buffer (Roche Diagnostics GmbH, Mannheim, Germany). Diluted samples and controls were added together with 100 μg/mL biotinylated anti-idiotypic capture antibody anti-ID CD20 (Roche Diagnostics GmbH) and 5 μg/mL Alexa-647 labelled anti-idiotypic detection antibody anti-ID TfR (Roche Diagnostics GmbH) by the Gyrolab xPlore instrument onto streptavidin-coated Gyrolab Bioaffy 1000 gyros discs (Gyros Protein Technologies AB). Using a 5% photomultiplier tube (PMT) setting, the response signals were proportional to the analyte concentration in the test sample. All study and control samples were analyzed in duplicates. The lower limit of quantification was 0.1 ng/ml in CSF and 10 ng/ml in serum samples.

### Quantification of RG6035 in Brain Lysates:

500 ng/ml biotinylated anti-ID CD20 antibody was transferred to the surface of a streptavidin-coated 96-well microtiter plate and incubated for 1 hour at room temperature. Unbound antibody was removed by washing followed by addition of brain lysate samples, diluted 1:100 in Roche Universal Buffer (Roche Diagnostics GmbH), and incubation for 1 hour. After removing unbound analyte by washing, detection was performed by successive addition of 500 ng/mL digoxygenin-conjugated anti-ID TfR antibody, incubation for 50 minutes at room temperature, washing and addition of 50 mU/mL anti-digoxygenin Fab fragments covalently bound to HRP. After a final washing step, 2,2′-azinobis [3-ethylbenzothiazoline-6-sulfonic acid]-diammonium salt (ABTS®) substrate solution was added, leading to a color change. The color intensity was photometrically determined (absorption at 405 nm; 490 nm reference wavelength) using an enzyme-linked immunoassay (ELISA) reader and was proportional to the amount of RG6035 in the test samples.

All study samples and control samples were analyzed in duplicates. The lower limit of quantification was 10 ng/mL.

### Quantification of the Blood Tracer in Brain Lysates:

500 ng/ml biotinylated anti-ID DP47 1 was transferred to the surface of a streptavidin-coated 96-well microtiter plate (Microcoat GmbH, Bernried, Germany) and incubated for 1 hour at room temperature. Unbound antibody was removed by washing followed by addition of brain lysate samples, diluted 1:100 in Roche Universal Buffer (Roche Diagnostics GmbH), and incubation for one hour. After removing unbound analyte by washing, detection was performed by successive addition of digoxygenin-conjugated anti-ID DP47 2 antibody (Roche Diagnostics GmbH) at 200 ng/mL, incubation for 50 min at room temperature, washing and addition of 25 mU/mL anti-digoxygenin Fab fragments covalently bound to HRP (Roche Diagnostics GmbH) at. After a final washing step, ABTS substrate solution was added. The color intensity of the reaction was photometrically determined (absorption at 405 nm; 490 nm reference wavelength) using an ELISA reader and was proportional to the amount of blood tracer in the test samples. All study samples and control samples were analyzed in duplicates. The lower limit of quantification was 18.75 ng/mL.

### Quantification of the Blood Tracer in Serum:

500 ng/mL biotinylated anti-ID DP47 1 was transferred to the surface of a streptavidin-coated 96-well microtiter plate (Microcoat GmbH, Bernried, Germany) and incubated for 1 hour at room temperature. Unbound antibody was removed by washing followed by addition of serum samples, diluted 1:1,000 in Roche Universal Buffer (Roche Diagnostics GmbH), and incubation for one hour. After removing unbound analyte by washing, detection was performed by successive addition of 200 ng/mL digoxygenin-conjugated anti-ID DP47 2 antibody (Roche Diagnostics GmbH), incubation for 50 min at room temperature, washing and addition of 25 mU/mL anti-digoxygenin Fab fragments covalently bound to HRP (Roche Diagnostics GmbH). After a final washing step, ABTS substrate solution was added. The color intensity of the reaction was photometrically determined (absorption at 405 nm; 490 nm reference wavelength) using an ELISA reader and was proportional to the amount of blood tracer in the test samples. All study samples and control samples were analyzed in duplicates. The lower limit of quantification was 187.5 ng/mL.

### PK Analysis:

Phoenix WinNonlin® (Certara, Princeton, NJ, USA) was used for non-compartmental (NCA) PK analysis. PK parameters for RG6035 in serum were calculated from the individual concentration data for the non-terminal groups Group 3, 4 and 5, dosed at 0.3 mg/kg, 1 mg/kg and 10 mg/kg, respectively. PK parameters for RG6035 in CSF were calculated from the composite concentration profiles of the CSF samples collected from the cisterna magna.

### Immunophenotyping:

For blood PD evaluation, 2 mL blood samples were used to assess B-cell depletion over time, as well as other lymphocyte subpopulations.

Immunophenotyping was used over 6 weeks to determine whether peripheral B-cells were activated (CD69 and CD86 surface markers) and whether specific B-cell subsets were preferentially depleted. Blood immunophenotyping and activation markers used were CD3, CD4, CD8, CD16, and CD19. The B-cell panel (blood and lymph node) used was CD45/CD1 and the activation markers used were CD69 and CD86.

### Immunophenotyping:

Samples from the spleen, a mesenteric and mandibular lymph node (LN) and tonsils were collected from all animals at the scheduled necropsy, using clean removal techniques to assess B-cell depletion. A slice in the middle of the spleen (approximately 1 cm thick) was taken and kept in a conical tube with 10 mL of assay medium (RPMI-1640 containing 5% (volume by volume (v/v)) fetal bovine solution (FBS)) and processed to single cell suspension. A portion of one tonsil and the LN were each taken and kept in a conical tube with 5 mL of assay medium (RPMI-1640 containing 5% (v/v) FBS) and processed to single cell suspension.

The cellular antigens and cell populations (Table 11) were quantified and reported as relative percentages for the spleen, lymph nodes and tonsils, as well absolute counts (cells/organ) for the spleen (hematology assessment (ADVIA® system, Siemens Healthineers, Erlangen, Germany)).

TABLE 11
Tissue biomarkers;

| | | Cell Population Identified^{a} |
|---|---|---|
| Antigen Marker(s) | | |
| CD45⁺ CD3⁻ CD19⁺ | | B-cells |
| CD45⁺ CD3⁺ | | Total T-cells |
| CD45⁺ CD3⁺ CD4⁺ | | T-helper cells |
| CD45⁺ CD3⁺ CD8⁺ | | T-cytotoxic cells |
| CD45⁺ CD3⁻ CD16⁺ | | Natural killer cells |
| CD45⁺ CD3⁻ CD19⁺ CD20⁺ | | CD20⁺ B-cells |
| Differentiation Antigen Marker(s) | | |
| CD45⁺ CD3⁻ CD19⁺ IgD⁺ CD27⁻ | | Naïve B-cells |
| CD45⁺ CD3⁻ CD19⁺ IgD⁺ CD27⁺ | | Unswitched memory B-cells |
| CD45⁺ CD3⁻ CD19⁺ IgD⁻ CD27⁺ | | CD27⁺ switched memory B-cells |
| CD45⁺ CD3⁻ CD19⁺ IgD⁻ CD27⁻ | | CD27⁻ switched memory B-cells |
| | Activation Antigen Marker(s) | Cell Population Identified^{a, b} |
| | CD45⁺ CD3⁻ CD19⁺ CD69⁺ | B-cell expression of CD69 |
| | CD45⁺ CD3⁻ CD19⁺ CD86⁺ | B-cell expression of CD86 |

^{a}Absolute count and percent of parental will be calculated and reported from indicated parent populations;
^{b}Median fluorescence intensity will be reported for CD69 as activation markers.

### Immunofluorescence Staining:

Formalin-fixed paraffin-embedded (FFPE) samples of tonsil, spleen, cervical and mandibular lymph nodes from all animals treated with RG6035 or vehicle treatment were sectioned at 2 μm onto Superfrost Plus slides. Tissue sections were baked overnight at 37° C. Stainings were performed using the Ventana Discovery Ultra automated tissue stainer (Ventana/Roche Tissue Diagnostics, Tucson, USA). Primary antibodies for immunofluorescence assays (anti-CD19 at 1:400, #ab182422, Abcam, Cambridge, UK; anti-CD20 at 1:200, #M0755, Dako, Agilent, Santa Clara, CA, USA) were diluted in Discovery Ab Diluent (#760-108, Ventana/Roche Tissue Diagnostics). Primary antibodies were detected using anti-species secondary antibodies conjugated to horseradish peroxidase (HRP; Roche Diagnostics GmbH) and tyramide signal amplification (TSA)-enhanced fluorescent kit (FAM or Cy5) was subsequently applied. Tissue was counterstained with DAPI (Roche Tissue Diagnostics) and mounted.

### Image Acquisition and Analysis:

Slides were digitized using Zeiss Axio Z.1 (whole slide scanner at 20× magnification; Zeiss, Oberkochen, Germany) resulting in single images for FAM, Cy5, and DAPI channels, respectively, and an overlay picture. Raw image data were saved in .czi format. Slides were viewed using the ZEN blue software (Zeiss). Pixel size was 0.27 pixel/μm in all images. Image analysis was performed with HALO AI 3.2. (IndicaLab, Albuquerque, NM, USA). A random forest classifier was used to detect tissue and exclude artefacts. Cell segmentation was performed using NucleiSeg artificial intelligence (AI) module. For the subsequent quantification of B-cells, the HighPlex FL v4.03 module was used to dynamically measure expression of the immunofluorescent marker within the previously segmented cells, using AI default nuclei segmentation (nuclear contrast threshold=0.5; minimum nuclear intensity=0.005; nuclear segmentation aggressiveness=0.2; maximum cytoplasm radius=1 μm; cell size=6-600 μm²). Positive and negative signals of each marker were determined in each organ with control stainings (vehicle animal; Alexa Fluor 488 (CD19) cytoplasm positive threshold=1400; 0% completeness; FIG. 27). Number of total cells, positive cells per marker and positive cells per phenotype were normalized by tissue area detected by the random forest tissue classifier.

### Immunocomplex Anti-Drug-Antibodies (ADA) Assay:

For the qualitative detection of antibodies directed against RG6035, a biotinylated capture antibody directed at the human Fc-region of RG6035, mAb<Hu-IgG>M-R10Z8E9 (Roche Diagnostics GmbH, Mannheim, Germany), was bound to a streptavidin-coated microtiter plate at 0.5 μg/mL and incubated for 1 hour at room temperature. After washing, samples and standards were diluted 1:100 in LowCross buffer (Candor Bioscience GmbH, Wangen im Allgaeu, Germany) spiked with 1 μg/mL RG6035. 100 μL were added to each well of the coated streptavidin-coated microtiter plate and incubated for 1 hour at room temperature. After washing, a digoxygenylated anti-cynomolgus monkey IgG detection antibody (Roche Diagnostics GmbH) at 0.5 μg/mL was added to the cavities of the microtiter plate and incubated for 1 hour. After washing, 25 mU/mL polyclonal anti-digoxygenin HRP conjugate was added and incubated for 1 hour. The peroxidase of the antibody-enzyme conjugate catalyzed the color reaction after addition of the substrate solution ABTS to the plate. Absorption was measured by an ELISA reader at 405 nm wavelength (reference wavelength: 490 nm). Twice the absorption signal of pre-dose samples of every individual cynomolgus monkey animal was set as the cut points to assess post-dose samples for ADAs.

### PK/PD Modeling in Cynomolgus Monkey:

The PK/PD modeling of the cynomolgus monkey PK in serum, the PK in brain and CSF, and the B-cell dynamics in blood was performed by non-linear mixed effects using the software version Monolix (Monolix 2019R2, http://lixoft.com/products/monolix/, Lixoft, Antony, France). Human projection model simulations were performed using MATLAB (MATLAB version R2020a, https://ch.mathworks.com/products/matlab.html, MathWorks, Natick, MA, USA).

In more detail, the PK of RG6035 was modeled using a two-compartment model, including a quasi equilibrium approximation of target-mediated drug disposition with a constant target pool.[24] Without being bound by this theory, it is assumed that this target-mediated disposition is most likely driven by the binding to CD20. An accelerated clearance term related to ADAs arising from an immune response against RG6035 was also added to the model.[28] Parameters were estimated using non-linear mixed-effects modeling (NLME) and individual (post-hoc) PK parameters were further used to calculate the PK input for the analyses of the RG6035 distribution into the CSF and brain and the PD of RG6035 on the B-cells in blood.

The distribution of RG6035 into the brain tissue and CSF was modeled by separate compartments for each brain region or CSF compartment. The outflow rate was assumed to be equal for all of these compartments. The inflow rate (multiplying the serum concentration) was estimated as the product of the outflow rate parameter and the equilibrium ratio to the serum concentration Kp (in analogy with the concept of a partition coefficient) which were estimated separately for each brain tissue and CSF compartment. A naïve pooled approach was taken for the estimation of the brain distribution parameters.

The model for the complex dynamics of blood B-cells was built on the concept of homeostatic balance between first-order cell loss and replacement at constant rate. RG6035 was assumed to stimulate the loss of cells from this main B-cell pool with a rate proportional to its concentration.[27,28] In order to consolidate the relatively rapid depletion observed in vitro and the long-lasting effects in vivo, it was further assumed that B-cells in blood are not directly represented by the main B-cell pool but are merely reflecting the depletion in the main pool with some delay. This was achieved by modeling the blood B-cells by another pool that is in slow exchange with the main pool.

Immediately after dosing, blood B cells show a rapid transient depletion but which seems nearly dose-independent. The model was extended to capture this first phase by a time-dependent (exponential) function, triggered by dosing events but otherwise independent of dose and exposure to RG6035.

All parameter estimations were performed using Monolix version 2020R1 (Lyxsoft).

### Human Projection:

The projection of the PK and PD of RG6035 in humans builds on the PK/PD modeling for cynomolgus monkey. For the PK, volumes were scaled with body weight, clearances with body weight to a power 0.85, and all rate parameters with a power −0.15 of the body weight.[23] An ad hoc projection of subcutaneous absorption in humans was modeled assuming a first-order process with parameter values in line with the expectations for this type of molecule (absorption rate of 0.02 h⁻¹ and fraction absorbed of 50%).[22] For the brain distribution, brain-to-serum concentration ratio of RG6035 was assumed to be equal between cynomolgus monkey and humans and the outflow rate was scaled allometrically with a power −0.15. Since immunogenicity in cynomolgus monkeys is not predictive for humans, its effects were not considered in the translation. Human projection of the PK/PD of B-cells in blood used in vitro data (EC50 of 1.68 nM in cynomolgus monkey versus 0.4 nM in humans) to account for species differences in potency.

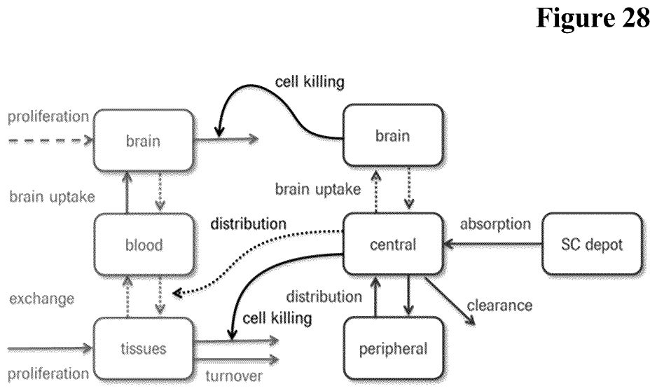

For ad hoc projections of PK/PD of B-cells in the brain, a corresponding B-cell pool was introduced into which B-cells migrate from blood and from which they leave at a rate assumed to be equal to the exchange rate of the two pools of the blood B-cell model. Without being bound by this theory, this seems consistent with published B-cell depletion from CSF after treatment with ocrelizumab when it is assumed that the depletion from CSF is the consequence of a reduced influx due to the depletion in blood.[44] Local B-cell killing is assumed to act with the same potency as in the periphery, however driven by the brain interstitial concentration of RG6035, calculated from projected brain tissue concentrations under the assumption that the compound is concentrated in the extracellular space and that the latter amounts to about 20% of total brain tissue.[45] The structure of the fully assembled model is illustrated in FIG. 28.

All model simulations for the human projection were performed using Matlab version 2020a (Mathworks).

1. RG6035 for use as a medicament in the treatment of multiple sclerosis, wherein RG6035 is administered at a dose of 140-210 mg.

2. RG6035 for use in the treatment of multiple sclerosis, wherein RG6035 is administered at a dose of 140-210 mg.

3. The use according to any one of claims 1 to 2, wherein the multiple sclerosis is primary progressive multiple sclerosis or secondary progressive multiple sclerosis.

4. The use according to any one of claims 1 to 3, wherein the dose is effective to deplete more than 95% of B-cells in the CSF of the subject that has been administered RG6035.

5. The use according to claim 4, wherein the depletion is achieved within 8 weeks after start of the administration.

6. The use according to any one of claims 1 to 5, wherein the concentration of RG6035 in the CSF is 1% or more of the serum concentration of RG6035.

7. The use according to any one of claims 1 to 6, wherein the concentration of RG6035 in the CSF is at least 0.055 μg/mL.

8. The use according to any one of claims 4 to 7, wherein the B-cells are CD19 positive B-cells.

9. The use according to any one of claims 1 to 8, wherein RG6035 is administered at a second dose after more than 95% of B-cells have been depleted from the CSF to maintain the B-cell depletion in the CSF at said level.

10. The use according to any one of claims 4 to 9, wherein the B-cell depletion is determined compared to the B-cell number prior to the first administration of RG6035.

11. The use according to any one of claims 1 to 10, wherein the administration is once every four weeks.

12. The use according to any one of claims 1 to 10, wherein the administration is started as once every week and changed to once every four weeks after more than 95% of B-cells have been depleted from the CSF to maintain the B-cell depletion in the CSF at said level.

13. The use according to any one of claims 1 to 12, wherein the administration of RG6035 is intravenously or subcutaneously.

14. The use according to any one of claims 1 to 13, wherein the administration of RG6035 is subcutaneously.

15. The use according to any one of claims 1 to 14, wherein RG6035 comprises a first polypeptide with the amino acid sequence of SEQ ID NO: 01, a second polypeptide with the amino acid sequence of SEQ ID NO: 02, a third polypeptide with the amino acid sequence of SEQ ID NO: 03 and a fourth polypeptide with the amino acid sequence of SEQ ID NO: 05.
